(19) United States
(12) Patent Application Publication
(10) Pub. No.: US 2023/0000974 A1
(43) Pub. Date: Jan. 5, 2023

(54) VIRUS-LIKE PARTICLE VACCINES

(71) Applicant: Verndari, Inc., Napa, CA (US)

(72) Inventors: Daniel R. HENDERSON, Napa, CA (US); Thomas J. ELLISON, Sacramento, CA (US); George C. TALBOTT, Sacramento, CA (US); Yeonju SONG, Woodland, CA (US)

(21) Appl. No.: 17/588,008

(22) Filed: Jan. 28, 2022

Related U.S. Application Data
(63) Continuation of application No. PCT/US2020/044196, filed on Jul. 30, 2020.
(60) Provisional application No. 62/990,318, filed on Mar. 16, 2020.
(60) Provisional application No. 62/880,547, filed on Jul. 30, 2019.

(51) Int. Cl. A61K 39/215; A61K 9/00; A61P 31/14; A61P 37/04

(52) U.S. Cl. CPC A61K 39/215; A61K 9/0021; A61P 31/14; A61P 37/04; A61K 2039/5258

(57) ABSTRACT

Provided, herein, in certain embodiments are virus-like particles such as synthetic enveloped VLPs or synthetic membrane VLPs. In some embodiments, the VLPs comprise a lipid bilayer. In some embodiments, the VLPs comprise a purified antigen anchored to the lipid bilayer. Some embodiments relate to vaccines comprising the VLP, methods of using the vaccine, and methods of making the vaccine or VLP.

### CROSS-REFERENCE TO RELATED APPLICATIONS

This application is the continuation of International Application No. PCT/US2020/044196, filed on Jul. 30, 2020, which claims the benefit of U.S. Provisional Application No. 62/880547 filed Jul. 30, 2019, and of U.S. Provisional Application No. 62/990318 filed Mar. 16, 2020, which applications are incorporated herein by reference in their entireties.

### SEQUENCE LISTING

The instant application contains a Sequence Listing which has been submitted electronically in ASCII format and is hereby incorporated by reference in its entirety. Said ASCII copy, created on Jan. 28, 2022, is named 47750_705_301 SL.txt and is 143 kilobytes in size.

### BACKGROUND

Diseases caused by infections are widespread. Many infectious diseases are difficult to prevent treat. For example, numbers of coronavirus infections such as coronavirus disease 2019 (COVID-19) are rising, and no cure is available. Better vaccines are needed to combat these diseases.

### SUMMARY

Disclosed herein, in certain embodiments, are virus-like particles (VLPs) comprising: (a) a synthetic or natural lipid bilayer; (b) an anchor molecule embedded in the lipid bilayer; and (c) an antigen bound to the anchor molecule. Disclosed herein, in certain embodiments, are VLPs comprising: (a) a synthetic lipid bilayer; (b) an anchor molecule embedded in the lipid bilayer; and (c) an antigen bound to the anchor molecule. In some embodiments, the lipid bilayer comprises a first lipid such as a phosphatidylcholine species. In some embodiments, the lipid bilayer comprises a second lipid such as a phosphatidylethanolamine species. In some embodiments, the first lipid and/or the second lipid each comprise an acyl chain comprising between 4 and 18 carbon atoms. In some embodiments, the first lipid and/or the second lipid each comprise four or less unsaturated bonds. In some embodiments, the first lipid of the lipid bilayer and/or the second lipid of the lipid bilayer are synthetic. In some embodiments, the lipid bilayer, the first lipid of the lipid bilayer, and/or the second lipid of the lipid bilayer are at least 99% pure, or are free or substantially free of biologic material. In some embodiments, the first lipid comprises 1,2-dioleoyl-sn-glycero-3-phosphocholine (DOPC). In some embodiments, the second lipid comprises 1,2-Dioleoyl-sn-glycero-3-phosphoethanolamine (DOPE). In some embodiments, the lipid bilayer comprises the first lipid and the second lipid at a predetermined ratio between 1:0.25 and 1:4. In some embodiments, the lipid bilayer comprises a sterol or sterol derivative. In some embodiments, the sterol or sterol derivative comprises cholesterol or DC-cholesterol. In some embodiments, the lipid bilayer comprises the sterol or sterol derivative at a ratio of 0-30 mol % in relation to the first lipid and/or the second lipid. In some embodiments, the antigen is at least 75.0%, 80.0%, 85.0%, 90.0%, 91.0%, 92.0%, 93.0%, 94.0%, 95.0%, 96.0%, 97.0%, 97.5%, 98.0%, 98.5%, 99.0%, 99.5%, 99.9%, 100%, or a range of percentages defined by any two of the aforementioned percentages, pure. In some embodiments, the antigen is bound directly to the anchor molecule, or wherein the antigen comprises the anchor molecule. In some embodiments, the antigen comprises a bacterial antigen, or a fragment thereof. In some embodiments, the bacterial antigen comprises an Actinomyces antigen, Bacillus antigens, e.g., immunogenic antigens from Bacillus anthracia, Bacteroides antigens, Bordetella antigens, Bartonella antigens, Borrelia antigens, e.g., B. burgdorferi OspA, Brucella antigens, Campylobacter antigens, Capnocytophaga antigens, Chlamydia antigens, Clostridium antigens, Corynebacterium antigens, Coxiella antigens, Dermatophilus antigens, Enterococcus antigens, Ehrlichia antigens, Escherichia antigens, Francisella antigens, Fusobacterium antigens, Haemobartonella antigens, Haemophilus antigens, e.g., H. influenzae type b outer membrane protein, Helicobacter antigens, Klebsiella antigens, L form bacteria antigens, Leptospira antigens, Listeria antigens, Mycobacteria antigens, Mycoplasma antigens, Neisseria antigens, Neorickettsia antigens, Nocardia antigens, Pasteurella antigens, Peptococcus antigens, Peptostreptococcus antigens, Pneumococcus antigens, Proteus antigens, Pseudomonas antigens, Rickettsia antigens, Rochalimaea antigens, Salmonella antigens, Shigella antigens, Staphylococcus antigens, Streptococcus antigens, e.g., S. pyogenes M proteins, Treponema antigens, and Yersinia antigens, e.g., Y. pestis F1 and V antigens. In some embodiments, the antigen comprises a fungal antigen, or a fragment thereof. In some embodiments, the fungal antigen comprises a Balantidium coli antigens, Entamoeba histolytica antigens, Fasciola hepatica antigens, Giardia lamblia antigens, Leishmania antigens, and Plasmodium antigens. In some embodiments, the antigen comprises a cancer antigen, or a fragment thereof. In some embodiments, the cancer antigen comprises tumor-specific immunoglobulin variable regions, GM2, Tn, sTn, Thompson-Friedenreich antigen (TF), Globo H, Le(y), MUC1, MUC2, MUC3, MUC4, MUCSAC, MUCSB, MUC7, carcinoembryonic antigens, beta chain of human chorionic gonadotropin (hCG beta), C35, HER2/neu, CD20, PSMA, EGFRvIII, KSA, PSA, PSCA, GP100, MAGE 1, MAGE 2, TRP 1, TRP 2, tyrosinase, MART-1, PAP, CEA, BAGE, MAGE, RAGE. In some embodiments, the antigen comprises a viral antigen, or a fragment thereof. In some embodiments, the viral antigen comprises an antigen from a human immunodeficiency virus (HIV), a flu virus, a Dengue virus, a Zika virus, a West Nile virus, an Ebola virus, Marburg virus, Rabies virus, a Middle Eastern respiratory syndrome (MERS) virus, a severe acute respiratory syndrome (SARS) virus, a respiratory syncytial virus (RSV), Nipah virus, human papilloma virus (HPV), Herpes virus, or a hepatitis virus, such as a hepatitis A (HepA) virus, a hepatitis B (HepB), or a hepatitis C (HepC) virus. In some embodiments, the antigen comprises an influenza protein, or a fragment thereof. In some embodiments, the influenza protein comprises a HA, NA, M1, M2, NS1, NS2, PA, PB1, or PB2 influenza protein, or a fragment thereof. In some embodiments, the influenza protein comprises an amino acid sequence that is 75.0%, 80.0%, 85.0%, 90.0%, 91.0%, 92.0%, 93.0%, 94.0%, 95.0%, 96.0%, 97.0%, 97.5%, 98.0%, 98.5%, 99.0%, 99.5%, 99.9%, 100%, or a range of percentages defined by any two of the aforementioned percentages, identical to any of SEQ ID NOs: 1-16, or a fragment thereof. In some embodiments, the influenza protein comprises an amino acid sequence that has no more than 1, 2, 3, 4, 5, 6, 7, 8, 9, 10, 20, 40, or a range defined by any of the aforementioned integers, amino acid substitutions, deletions, and/or insertions, compared to any of SEQ ID NOs: 1-16, or a fragment thereof. In some embodiments, the influenza protein is encoded by a nucleic acid with a sequence that is 75.0%, 80.0%, 85.0%, 90.0%, 91.0%, 92.0%, 93.0%, 94.0%, 95.0%, 96.0%, 97.0%, 97.5%, 98.0%, 98.5%, 99.0%, 99.5%, 99.9%, 100%, or a range of percentages defined by any two of the aforementioned percentages, identical to a nucleic acid sequence encoding any of amino acid SEQ ID NOs: 1-16, or a fragment thereof. In some embodiments, the influenza protein is encoded by a nucleic acid with a sequence that has no more than 1, 2, 3, 4, 5, 6, 7, 8, 9, 10, 20, 40, or a range defined by any of the aforementioned integers, nucleic acid substitutions, deletions, and/or insertions, compared to a nucleic acid sequence encoding any of amino acid SEQ ID NOs: 1-16, or a fragment thereof. In some embodiments, the antigen comprises a coronavirus protein, or a fragment thereof. In some embodiments, the coronavirus comprises a severe acute respiratory syndrome coronavirus 2 (SARS-CoV-2). In some embodiments, the coronavirus protein comprises a spike (S) protein, an envelope (E) protein, a membrane protein (M), or a nucleocapsid (N) protein. In some embodiments, the coronavirus protein comprises S1 or S2. In some embodiments, the coronavirus protein comprises an amino acid sequence that is 75.0%, 80.0%, 85.0%, 90.0%, 91.0%, 92.0%, 93.0%, 94.0%, 95.0%, 96.0%, 97.0%, 97.5%, 98.0%, 98.5%, 99.0%, 99.5%, 99.9%, 100%, or a range of percentages defined by any two of the aforementioned percentages, identical to any of SEQ ID NOs: 20-29, or a fragment thereof. In some embodiments, the coronavirus protein comprises an amino acid sequence that has no more than 1, 2, 3, 4, 5, 6, 7, 8, 9, 10, 20, 40, or a range defined by any of the aforementioned integers, amino acid substitutions, deletions, and/or insertions, compared to any of SEQ ID NOs: 20-29, or a fragment thereof. In some embodiments, the anchor molecule comprises a transmembrane protein, a lipid-anchored protein, or a fragment or domain thereof. In some embodiments, the anchor molecule comprises a hydrophobic moiety. In some embodiments, the anchor molecule comprises a prenylated protein, fatty acylated protein, a glycosylphosphatidylinositol-linked protein, or a fragment thereof. In some embodiments, the VLP further comprises a synthetic lipid vesicle comprising the lipid bilayer. In some embodiments, the lipid bilayer comprises an inner surface and an outer surface. In some embodiments, the antigen is presented on the outer surface of the lipid vesicle. In some embodiments, the antigen is presented on the inner surface of the lipid vesicle. In some embodiments, the VLP is a seVLP and the lipid bilayer is in the form of a synthetic lipid vesicle. In some embodiments, the VLP is in the form of a synthetic membrane virus-like particle (smVLP) comprising a nanodisc. In some embodiments, the nanodisc has a diameter of between 5-200 nM. In some embodiments, the nanodisc comprises an amphiphilic polymethacrylate (PMA) copolymer. In some embodiments, the nanodisc comprises styrene-maleic acid lipid particles (SMALPs). In some embodiments, the nanodisc comprises a diisobutylenemaleic acid (DIBMA) co-polymer. In some embodiments, the PMA copolymer is toroidal. In some embodiments, the SMALPs are toroidal. In some embodiments, the DIBMA co-polymer is toroidal. In some embodiments, the nanodisc comprises an amphiphilic toroidal polymethacrylate (PMA) copolymer, SMALP, or DIBMA co-polymer.

Disclosed herein, in certain embodiments, are vaccines comprising: a VLP as described herein, and a pharmaceutically acceptable excipient, carrier, and/or adjuvant. In some embodiments, the excipient comprises an anti-adherent, a binder, a coating, a color or dye, a disintegrant, a flavor, a glidant, a lubricant, a preservative, a sorbent, a sweetener, or a vehicle. In some embodiments, the vaccine comprises the adjuvant. In some embodiments, the adjuvant comprises a Toll-like receptor (TLR) agonist such as imiquimod, Flt3 ligand, monophosphoryl lipid A (MLA), or an immunostimulatory oligonucleotide such as a CpG oligonucleotide. In some embodiments, the adjuvant comprises imiquimod. In some embodiments, the vaccine is formulated in a solvent or liquid such as a saline solution, a dry powder, or as a sugar glass. In some embodiments, the vaccine is lyophilized. In some embodiments, the vaccine is formulated for intranasal, intradermal, intramuscular, topical, oral, subcutaneous, intraperitoneal, intravenous, or intrathecal administration. In some embodiments, the vaccine comprises a dose of 1 pg, 10 pg, 25 pg, 100 pg, 250 pg, 500 pg, 750 pg, 1 ng, 5 ng, 10 ng, 15 ng, 20 ng, 25 ng, 50 ng, 100 ng, 250 ng, 500 ng, 1μg, 10 μg, 50 μg, 100 μg, 500 μg, 1 mg, 5 mg, 10 mg, 50 mg, 100 mg, 500 mg, or 1 g of the seVLP, or a range of doses defined by any two of the aforementioned doses. In some embodiments, the vaccine comprises a dose of 25 pL, 50 pL, 100 pL, 250 pL, 500 pL, 750 pL, 1 nL, 5 nL, 10 nL, 15 nL, 20 nL 25 nL, 50 nL, 100 nL, 250 nL, 500 nL, 1 μL, 10 μL, 50 μL, 100 μL, 500 μL, 1 mL, or 5 mL of the vaccine, or a range of doses defined by any two of the aforementioned doses. In some embodiments, the vaccine is formulated for microneedle administration in a 100 pL-20 nL dose. In some embodiments, the dose is on or in each microneedle of a microneedle device. In some embodiments, the vaccine is formulated as a trehalose sugar glass.

Disclosed herein, in certain embodiments, are VLPs, comprising: (a) a synthetic lipid bilayer comprising a first lipid and a second lipid; (b) an anchor molecule embedded in the lipid bilayer; and (c) a SARS-CoV-2 protein bound to the anchor molecule. In some embodiments, first lipid comprises a phosphatidylcholine species. In some embodiments, the first lipid comprises DOPC. In some embodiments, the second lipid comprises a phosphatidylethanolamine species. In some embodiments, the second lipid comprises DOPE. In some embodiments, the lipid bilayer comprises the first lipid and the second lipid at a predetermined ratio between 1:0.25 and 1:4. In some embodiments, the lipid bilayer further comprises cholesterol or DC-cholesterol, or a derivative thereof In some embodiments, the lipid bilayer comprises the cholesterol or DC-cholesterol, or a derivative thereof at a ratio of 0-30 mol % in relation to the first lipid or the second lipid. In some embodiments, the SARS-CoV-2 protein is bound directly to the anchor molecule, or wherein the SARS-CoV-2 protein comprises the anchor molecule. In some embodiments, the SARS-CoV-2 protein comprises a spike protein. In some embodiments, the spike protein comprises Si or S2. In some embodiments, the spike protein comprises an amino acid sequence that is at least 85% identical to SEQ ID NO: 25. In some embodiments, the spike protein comprises an amino acid sequence that has no more than 10 amino acid substitutions, deletions, or insertions, compared to SEQ ID NO: 25. In some embodiments, the spike protein binds to a human angiotensin converting enzyme 2 (ACE2). Disclosed herein, in certain embodiments, are vaccines comprising the VLP, and a pharmaceutically acceptable excipient, carrier, or adjuvant. In some embodiments, the adjuvant comprises imiquimod. In some embodiments, the vaccine is formulated for injection by a microneedle. In some embodiments, the vaccine is lyophilized. In some embodiments, the vaccine is formulated as a sugar glass. Disclosed herein, in certain embodiments, are vaccination methods comprising administering the vaccine to a subject in need thereof

Disclosed herein, in certain embodiments, are synthetic enveloped virus-like particles (seVLPs), comprising: (a) a synthetic lipid vesicle comprising a lipid bilayer having an inner surface and an outer surface; (b) an anchor molecule embedded in the lipid bilayer; and (c) a SARS-CoV-2 protein bound to the anchor molecule. In some embodiments, the SARS-CoV-2 protein is presented on the outer surface of the lipid vesicle. In some embodiments, the SARS-CoV-2 protein is presented on the inner surface of the lipid vesicle. In some embodiments, the SARS-CoV-2 protein comprises an S1 or S2 spike protein. In some embodiments, the seVLP is formulated as a sugar glass for injection.

Disclosed herein, in certain embodiments, are smVLPs, comprising: (a) a synthetic nanodisc comprising a lipid bilayer comprising an inner surface and an outer surface; (b) an anchor molecule embedded in the lipid bilayer; and (c) a SARS-CoV-2 protein bound to the anchor molecule. In some embodiments, the nanodisc comprises a 5-200 nM diameter. In some embodiments, the nanodisc comprises an amphiphilic toroidal polymethacrylate (PMA) copolymer, SMALP, DIBMA co-polymer, or non-immunogenic mimetic peptides of an alpha helix of ApoA. In some embodiments, the SARS-CoV-2 protein comprises an S1 or S2 spike protein. In some embodiments, the smVLP is formulated as a sugar glass for injection.

Disclosed herein, in certain embodiments, are microneedle devices loaded with a vaccine as described herein. In some embodiments, the microneedle device comprises a substrate comprising a sheet and a plurality of microneedles extending therefrom. In some embodiments, the vaccine is formulated in a sugar glass. In some embodiments, the sugar glass is trehalose. In some embodiments, the microneedle device comprises a metal snap applicator fastened by tape to a support material.

Disclosed herein, in certain embodiments, are methods of making a seVLP, comprising: microfluidically combining (i) an aqueous solution comprising an antigen bound to an anchor molecule with (ii) an ethanolic solution comprising a first lipid and a second lipid, thereby mixing the aqueous solution with the ethanolic solution to form a seVLP comprising a lipid bilayer comprising the first and second lipids with the anchor molecule embedded in the lipid bilayer. In some embodiments, microfluically combining the aqueous solution with the ethanolic solution comprises mixing a stream of the aqueous solution with a stream of the ethanolic solution.

Disclosed herein, in certain embodiments, are methods for preventing, reducing the occurrence of, or reducing the severity of a disease, comprising: administering a vaccine as described herein, to a subject; wherein the administration prevents, reduces the occurrence of, or reduces the severity of the disease. In some embodiments, the disease comprises an infection. In some embodiments, the disease comprises a bacterial, fungal, or viral infection. In some embodiments, the viral infection comprises an influenza infection. In some embodiments, the viral infection is a coronavirus infection. In some embodiments, the viral infection is coronavirus disease 2019 (COVID 19). In some embodiments, the subject is a mammal or human subject. In some embodiments, the administration comprises administration by one or more needles or microneedles. In some embodiments, the administration comprises administration by a pre-formed liquid syringe. In some embodiments, the administration comprises intranasal, intradermal, intramuscular, skin patch, topical, oral, subcutaneous, intraperitoneal, intravenous, or intrathecal administration. In some embodiments, the administration comprises administering a dose of 1 pg, 10 pg, 25 pg, 100 pg, 250 pg, 500 pg, 750 pg, 1 ng, 5 ng, 10 ng, 15 ng, 20 ng, 25 ng, 50 ng, 100 ng, 250 ng, 500 ng, 1μg, 10 μg, 50 μg, 100 μg, 500 μg, 1 mg, 5 mg, 10 mg, 50 mg, 100 mg, 500 mg, or 1 g of the seVLP or vaccine, or a range of doses defined by any two of the aforementioned doses. In some embodiments, 100 pL-20 nL of the vaccine is administered by each microneedle. In some embodiments, 5-20 nL of the vaccine is administered by each microneedle. In some embodiments, the vaccine is administered using a microneedle device as described herein.

Disclosed herein, in certain embodiments, are kits comprising: a microneedle loaded with a VLP or vaccine as described; and a wipe, a desiccant, and/or a bandage. In some embodiments, the kit comprises a microneedle device as described herein. In some embodiments, the kit contains an imiquimod wipe.

Disclosed herein, in certain embodiments, are methods for determining an effectiveness of a vaccine, comprising: obtaining a sample obtained from a subject who has been administered a vaccine, the sample comprising a presence or an amount of a virus; providing a substrate comprising an ACE2 or fragment thereof capable of binding to a virus protein; contacting the substrate with the sample to bind virus or protein virus in the sample to the ACE2 or fragment thereof; detecting virus or protein virus bound to the ACE2 or fragment thereof of the substrate; and determining the presence or amount of the virus in the sample based on the detected virus or protein virus bound to the ACE2 or fragment thereof of the substrate, thereby determining the effectiveness of the vaccine. In some embodiments, the sample is from a subject. In some embodiments, the sample comprises blood, serum, or plasma. In some embodiments, the virus is a coronavirus. In some embodiments, the virus is a SARS-CoV-2. In some embodiments, the virus protein is a SARS-CoV-2 spike protein. In some embodiments, the amount of virus in the sample is decreased compared to another sample obtained from the subject before the subject was administered the vaccine. In some embodiments, the amount of virus in the sample is increased compared to another sample obtained from the subject before the subject was administered the vaccine. Some embodiments further comprise recommending or providing a virus treatment to the subject based on the amount of the virus in the sample or the effectiveness of the vaccine. In some embodiments, the virus treatment comprises a coronavirus treatment such as a COVID-19 treatment. In some embodiments, the vaccine comprises a VLP.

Disclosed herein, in certain embodiments, are methods for determining an effectiveness of a vaccine, comprising: obtaining a sample obtained from a subject who has been administered a vaccine, the sample comprising a presence or an amount of anti-virus antibodies; providing a substrate comprising a virus protein or fragment thereof capable of binding to the anti-virus antibodies; contacting the substrate with the sample to bind anti-virus antibodies in the sample to the virus protein or fragment thereof; detecting anti-virus antibodies bound to the virus protein or fragment thereof of the substrate; and determining the presence or amount of the anti-virus antibodies in the sample based on the detected anti-virus antibodies bound to the virus protein or fragment thereof of the substrate, thereby determining the effectiveness of the vaccine. In some embodiments, the sample is from a subject. In some embodiments, the sample comprises blood, serum, or plasma. In some embodiments, the virus is a coronavirus. In some embodiments, the virus is a SARS-CoV-2. In some embodiments, the virus protein is a SARS-CoV-2 spike protein. In some embodiments, the amount of anti-virus antibodies in the sample is decreased compared to another sample obtained from the subject before the subject was administered the vaccine. In some embodiments, the amount of anti-virus antibodies in the sample is increased compared to another sample obtained from the subject before the subject was administered the vaccine. Some embodiments further comprise recommending or providing a virus treatment to the subject based on the amount of the anti-virus antibodies in the sample or the effectiveness of the vaccine. In some embodiments, the virus treatment comprises a coronavirus treatment such as a COVID-19 treatment. In some embodiments, the vaccine comprises a VLP.

Disclosed herein, in certain embodiments, are virus-like particle VLPs, comprising: a synthetic lipid bilayer comprising a first lipid and a second lipid; an anchor molecule embedded in the lipid bilayer; and a SARS-CoV-2 protein bound to the anchor molecule. In some embodiments, the first lipid comprises a phosphatidylcholine species. In some embodiments, wherein the first lipid comprises DOPC. In some embodiments, the second lipid comprises a phosphatidylethanolamine species. In some embodiments, the second lipid comprises DOPE. In some embodiments, the lipid bilayer comprises the first lipid and the second lipid at a predetermined ratio between 1:0.25 and 1:4. In some embodiments, the lipid bilayer further comprises cholesterol or DC-cholesterol, or a derivative thereof. In some embodiments, the lipid bilayer comprises the cholesterol or DC-cholesterol, or a derivative thereof at a ratio of 0-30 mol % in relation to the first lipid or the second lipid. In some embodiments, the SARS-CoV-2 protein is bound directly to the anchor molecule, or wherein the SARS-CoV-2 protein comprises the anchor molecule. In some embodiments, the SARS-CoV-2 protein comprises a spike protein. In some embodiments, the spike protein comprises S1 or S2. In some embodiments, the spike protein comprises an amino acid sequence that is at least 85% identical to SEQ ID NO: 25. In some embodiments, the spike protein comprises an amino acid sequence that has no more than 10 amino acid substitutions, deletions, or insertions, compared to SEQ ID NO: 25. In some embodiments, the spike protein binds to an ACE2. In some embodiments, a vaccine comprising the VLP, and a pharmaceutically acceptable excipient, carrier, or adjuvant. In some embodiments, the adjuvant comprises imiquimod. In some embodiments, the vaccine is formulated for injection by a microneedle. In some embodiments, the vaccine is lyophilized. In some embodiments, the vaccine is formulated as a sugar glass. Some embodiments comprise a vaccination method comprising administering the vaccine to a subject in need thereof

Disclosed herein, in certain embodiments, are seVLPs, comprising: a synthetic lipid vesicle comprising a lipid bilayer having an inner surface and an outer surface; an anchor molecule embedded in the lipid bilayer; and a SARS-CoV-2 protein bound to the anchor molecule. In some embodiments, the SARS-CoV-2 protein is presented on the outer surface of the lipid vesicle. In some embodiments, the SARS-CoV-2 protein is presented on the inner surface of the lipid vesicle. In some embodiments, the SARS-CoV-2 protein comprises an S1 or S2 spike protein. In some embodiments, the seVLPs are formulated as a sugar glass for injection.

Disclosed herein, in certain embodiments, are smVLPs, comprising: a synthetic nanodisc comprising a lipid bilayer comprising an inner surface and an outer surface; an anchor molecule embedded in the lipid bilayer; and a SARS-CoV-2 protein bound to the anchor molecule. In some embodiments, the nanodisc comprises a 5-200 nM diameter. In some embodiments, the nanodisc comprises an amphiphilic toroidal polymethacrylate (PMA) copolymer, styrene-maleic acid lipid particle (SMALP), DIBMA co-polymer, or non-immunogenic mimetic peptides of an alpha helix of ApoA. In some embodiments, the SARS-CoV-2 protein comprises an S1 or S2 spike protein. In some embodiments, the smVLP is formulated as a sugar glass for injection.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 is a diagram of some examples of antigens;

FIG. 2 is a flow diagram illustrating an example of a method for preparing an antigen;

FIG. 3 is a chart illustrating data related to antigen purification in accordance with some embodiments;

FIG. 4 is a western blot image showing eluted antigens in accordance with some embodiments;

FIG. 5 includes a table and chart illustrating the sizes and volumes of some liposomes;

FIG. 6 includes charts showing data related to liposome preparation in accordance with some embodiments;

FIG. 7 is a magnified image of some microneedles;

FIG. 8 is a chart illustrating ELISA data in accordance with some embodiments;

FIG. 9 includes images of an example of a microneedle device;

FIG. 10 is a schematic drawing of an example of a VaxiPatch;

FIG. 11 is an image showing the front and back of an example of a kit that includes a vaccine as described herein;

FIG. 12 is an image showing insertion of a printed array into a bending jig in an example process for making a microneedle device;

FIG. 13 is an image showing a metal snap applicator attached to a support material in an example process for making a microneedle device;

FIG. 14 shows an example three-pronged approach to address the point-of-care vaccination problem;

FIGS. 15A and 15B show example sheets of microneedle arrays;

FIG. 16 shows an example of a vaccine loaded microarray;

FIG. 17 shows an example of a VaxiPatch dye delivery in five minutes in a human subject;

FIG. 18 shows an example of a VaxiPatch dye delivery in a rat;

FIG. 19 shows VaxiPatch Rat ELISA titers with an IgG timecourse;

FIG. 20 shows VaxiPatch ELISA titers to B/Colorado 2017;

FIG. 21 shows Hemagglutination inhibition titers to B/Colorado 2017 dot plot;

FIG. 22 shows a bar graph representation of HAI data;

FIG. 23 shows VaxiPatch VMLP accelerated stability of antigen studies;

FIG. 24 shows that COGS are lower than industry average;

FIG. 25 shows an example chart with enveloped glycoprotein subunit vaccines;

FIG. 26 shows a Vaccine Pipeline introduction;

FIG. 27 shows an example COVID-S expression in ExpiCHO;

FIG. 28 shows an example COVID spike western blot that confirms the identity for recombinant COVID-S protein;

FIG. 29 shows a full-length spike purification with an elution profile of IMAC purification of COVID-S;

FIG. 30 shows a COVID-19 spike lentivirus pseudotype construction;

FIG. 31 depicts an example Coomassie stained SDS-PAGE gel showing samples from a purification;

FIG. 32 depicts an example of levels of activity in the ACE-2 samples;

FIG. 33 depicts an example linear regression of the data for this experiment.

FIG. 34 depicts an example standard curve from a test of the ability of VrS01 to bind 250 ng of ACE-2 over four different concentrations;

FIG. 35A depicts results from an example experiment where the stability of the VrS01 was tested at different temperatures;

FIG. 35B depicts the amount of potent VrS01 remaining determined based on converting the absorbance values;

FIG. 36 depicts an example linear regression for “print mix” VMLPs;

FIG. 37 depicts a graph of the ACE-2 binding at different pH levels is displayed;

FIG. 38 depicts a bar graph with a plot of the average absorbance;

FIG. 39 shows a summary diagram of the VRS01 construct; and

FIG. 40 shows specific IgG responses to VrS01 in SD rats.

### DETAILED DESCRIPTION

Disclosed herein, in certain embodiments, are seVLPs comprising: (a) a synthetic lipid vesicle comprising a lipid bilayer comprising an inner surface and an outer surface; (b) an anchor molecule embedded in the lipid bilayer; and (c) an antigen bound to the anchor molecule. Also disclosed herein, in certain embodiments, are smVLPs comprising a nanodisc comprising a synthetic, semisynthetic or natural lipid bilayer comprising an inner surface and an outer surface; an anchor molecule embedded in the lipid bilayer; and an antigen bound to the anchor molecule. Disclosed herein, in certain embodiments, are vaccines comprising a seVLP or smVLP, and methods for their use and manufacturing.

A benefit of some vaccines described herein is that they are cost-effective and safer than traditional vaccines or vaccines on the market. Some preventative viral vaccines on the market are based on inactivated or live-attenuated viruses. Formalin killed or inactivated polio, (Ipol®, Sanofi) and influenza (flu) (Afluria®, Seqiris; Fluzone®, Sanofi) vaccines are examples of inactivated viral vaccines, while the live-attenuated measles, mumps and rubella (MMR-II®, Merck) vaccines are examples of live-attenuated viral vaccines.

### I. OVERVIEW

The VLPs described herein have been developed to fill the need for providing a vaccine that is more cost-effective, safer, or faster to make than a traditional vaccine. VLPs are non-infectious particles resembling their parental viruses. In some embodiments, VLPs have antigens of their parental viruses, or have antigens that are similar to their parental viruses. In some embodiments, antigenic proteins of VLPs are produced in bacterial, yeast, insect, plant or mammalian expression systems by recombinant DNA methods. Beyond safety, another benefit of some VLPs is that they present the antigenic proteins in a structural array that are more easily recognized by pathogen associated molecular pattern recognition receptors (PAMPs) such as TLRs than other vaccines. In this way VLPs become an adjuvant to the antigenic proteins, in some embodiments. As a result, in some embodiments, VLPs are more immunogenic than individual soluble proteins of which they are composed.

Some non-enveloped VLP vaccines include commercial vaccines for Hepatitis B (Engerix-B®, GSK) produced in yeast and HPV; Gardasil® 9, Merck; Cevarix®, GSK) produced in yeast and insect cells respectively, and these vaccines have a single protein, HBsAg of Hepatitis B virus and L1 of HPV that spontaneously form an empty icosahedral capsid shell. Some additional non-enveloped VLP vaccines include Hepatitis E virus (HEV) (Hercolinl®, Xiamen Innovax Biotech Co., China) produced in E. coli, Malaria (Mosquirix®, GSK) produced in insect cells, and two second generation Hepatitis B vaccines (Sci-B-Vac®, VBI Vaccines, Inc. and HEPLISAV-B®, Dynavax).

Some VLPs are enveloped (eVLPs). eVLPs are more complex than nonenveloped VLPs in that they contain lipids derived from the expression system in which they are produced as well as one or more of the immunogenic proteins from the parental virus. These eVLPs get their lipid membrane from budding off of their host cells. For example, such eVLPs have been for HIV, Influenza, Chickungunya, SARS, Nipah, Ebola, Dengue, Rift Valley fever and Lassa virus. These eVLPs were produced in yeast, insect cells, mammalian cells and plants. But none of these eVLP vaccines have reached commercial production.

Problems have prohibited the commercial use of eVLPs and other vaccines. For example, a commercial eVLP vaccine is Inflexal®, an influenza vaccine. To produce Inflexal, influenza virus is grown in chicken eggs. Virions containing the hemagglutinin (HA) and neuroaminidase (NA) glycoproteins were solubilized with the detergent octaethylene glycol mono (n-dodecyl) ether, the nucleocapsid was removed by centrifugation, and the resulting crude undefined supernatant mixture was supplemented 10% with additional external phospholipids. These eVLPs were produced by mixing and removal of detergent. Inflexal was introduced in the European market in 1997. The cost of goods was a problem for Inflexal. In 2012 two contaminated lots of Inflexal were shipped from Switzerland to Italy, and the production of Inflexal was ended. These eVLPs contained egg derived protein and lipid contaminants, an undefined ratio of influenza HA and NA and an unknown amount of influenza M2. The mixing process and detergent removal producing eVLPs is poorly defined leading to the contamination that ended production.

Thus, existing eVLPs have problems that limit their success. Some eVLPs are less stable than single protein capsid VLPs due to the lipid membrane. Some eVLPs are produced in lower yield in expression systems as they form by budding off the producer cells. Some eVLPs are contaminated by host cell proteins encapsulated within the eVLP in the process of budding from the cells of the expression system. Some eVLPs produced in the insect cell system are contaminated by baculovirus particles of near identical size and morphology. Some eVLPs are difficult to purify often requiring ultracentrifugation through sucrose gradients. Some embodiments of the vaccines described herein have solved one or more of these issues and provide a solution to the long felt need in the art for improved vaccines that are safe, free of contaminants, and effective.

Previous vaccines have not included fully synthetic vesicles clean of other proteins or lipids derived from eggs. Making synthetic enveloped VLPs or vaccines solves the problem of the undefined nature of current VLP vaccines made from cells. In some embodiments, the vaccines provided herein are developed or produced quickly, whereas previous influenza vaccines, for example, took too long to develop or were too expensive to make to be fully effective during a particular flu season.

Influenza A is responsible for up to half a million deaths worldwide each year. Although several subtypes commonly circulate in humans, in some embodiments new subtypes are introduced at any time through zoonotic infection. In some embodiments, the zoonotic infection comprises H5N1 or H7N9. Even though the seasonal vaccine is updated every year, these zoonotic transmissions are unpredictable and not accounted for in the vaccine. Currently available vaccines are not sufficient because (1) inactivated vaccines do not generate a robust mucosal immune response, and (2) live attenuated influenza vaccines (LAIV) are problematic because they are over-attenuated, have restricted usage guidelines, and LAIV with HA and NA subtypes not present in seasonal strains cannot be used because of the risk of reassortment with wild type viruses. Currently available vaccines are designed to be protective against specific strains and reformulated every year and do not provide universal protection. Specific pre-pandemic vaccines, both inactivated and LAIV, against avian influenza viruses have not been very immunogenic. A universal vaccine aimed to stem zoonotic influenza infections from becoming pandemics could supplement the current seasonal vaccine and would be beneficial to public health. In some embodiments, a universal vaccine protects against all avian subtypes, against 16 avian HA subtypes (H1 to H16) or is manufactured quickly in the event of a pandemic.

In some embodiments, the VLPs comprise a polyvalent mixture of influenza seVLPs each containing a single influenza A HA subtype (or a single NA subtype) to avoid a problem of immunodominance of HA over NA. In some embodiments, the VLPs are seVLPs or smVLPs containing influenza A NA proteins. In some embodiments, the VLPs comprise two or more different antigens, for example influenza A NA proteins and influenza A matrix proteins, such as M1, M2, or both. These polyvalent VLPs are non-infectious, safe, and easy to manufacture and use. In some embodiments, these polyvalent VLPs are used to provide a broadly protective ‘universal’ pre-pandemic vaccine and a more broadly reactive seasonal vaccine.

In some embodiments, the vaccines are delivered intranasally, intramuscularly, intradermally, systemically, or intravenously to elicit broadly reactive immunity to conserved epitopes on the influenza virus HA head and stalk as well as to NA epitopes and thus to confer protection to a wide range of influenza A viruses. In some embodiments, although HA is antigenically diverse, conserved epitopes in the HA receptor binding and stalk domains allow cross-reactive vaccines to be produced.

In some embodiments, a subunit vaccine against SARS-CoV-2 is developed by expressing a recombinant SARS-CoV-2 spike protein in a mammalian cell line, purifying the protein, and formulating it into membrane bound particles (VMLP) to be used in combination with a dual adjuvant system. In some embodiments, aspects in the development of a subunit vaccine include determine a potency of the antigen used in the vaccine. To this end, the natural cellular receptor target of SARS-CoV-2, angiotensin converting enzyme 2 (ACE-2), may be leveraged in a sandwich enzyme-linked immunosorbent assay (ELISA). The ability of SARS-CoV-2-S to bind ACE-2 can be quantified with this assay and used as an indicator of SARS-CoV-2-S potency. In some embodiments, stability of the SARS-CoV-2-S is measured over time, in different storage conditions or in different formulations.

In some embodiments, modifications of the sandwich ELISA can also be used as a measure of whether a subunit vaccine has elicited an efficacious immune response. The ability of antibodies to neutralize the binding of SARS-CoV-2-S to ACE-2 is shown herein to correlate with protective immune responses. As a result, assays described herein can be used to screen people to see if they have SARS-Cov-2 neutralizing antibody (NAb). In addition, the amount of NAb can be measured and correlated with the level of NAb required to protect people from COVID-19. Currently, NAb is measured biologically with either live SARS-CoV-2 virus (BSL3 required and high coefficient of variation, (CV)) or with pseutotyped virus such as VSV expressing a reporter gene and the SARS-CoV-2 spike glycoprotein (BSL2 required and high CV). An improvement described herein turns the NAb test into a simple BLS1 quantitative immunoassay with a low CV. Commercial immunoassays in various formats are envisioned.

In some embodiments, to develop the sandwich ELISA, a mammalian expression vector is commissioned to generate the ectodomain of ACE-2 corresponding to the first 740 amino acids (SEQ ID NO: 17) of the protein (SEQ ID NO: 18). In some embodiments, ACE-2 was purified using ion-exchange chromatography and tested to determine whether it had retained its enzymatic activity using a fluorogenic substrate assay. In some embodiments, high-binding ELISA plates were coated with ACE-2 overnight, blocked with bovine serum albumin and then incubated with different concentrations of SARS-CoV-2-S to determine the linear range of the assay. In some embodiments, an “in-house” SARS-CoV-2-S (VrS01) was compared to commercially available SARS-CoV-2-S. In some embodiments, to ensure binding to ACE-2 was specific to the SARS-CoV-2-S, binding was compared to “in-house” hemagglutinin. In some embodiments, heat stress, pH stress, and commercially available polyclonal antibody raised against the S1 domain of SARS-CoV-2-S were tested for their ability to affect SARS-CoV-2-S/ACE-2 binding.

In some embodiments, purified recombinant ACE-2 can be used for the capture step of a sandwich ELISA used to test the potency of recombinant SARS-CoV-2-S as a vaccine antigen. The binding interaction between these molecules is disrupted when SARS-CoV-2-S has been stressed with pH or heat, suggesting this assay is sensitive to changes in the quality and conformation of SARS-CoV-2-S. There is a linear relationship in the binding interaction with ACE-2 over a large range of SARS-CoV-2-S concentrations, and when recombinant SARS-CoV-2-S is incorporated into membrane bound particles, the ACE-2 binding relationship remains linear and is not inhibited by other components of the vaccine formulation. Binding to ACE-2 is specific to SARS-CoV-2-S, as hemagglutinin from the B/Colorado '17 strain of influenza does not bind to ACE-2 when assayed at the same concentrations. Finally, a commercially available polyclonal antibody raised against the Si subunit of SARS-CoV-2-S may inhibit binding to ACE-2. Thus, an ACE-2 binding based sandwich ELISA is a powerful tool in determining SARS-CoV-2-S potency and/or stability and has utility in determining whether sera from vaccinated individuals have neutralizing antibodies. Non-limiting examples of some such embodiments are included in Examples 12-16.

### II. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, “administering” a vaccine to a subject comprises giving, applying or bringing the vaccine into contact with the subject. In some embodiments, administration is accomplished by any of a number of routes. In some embodiments, administration is accomplished by a topical, oral, subcutaneous, intramuscular, intraperitoneal, intravenous, intrathecal or intradermal route.

As used herein, an “antibody” is in some embodiments an immunoglobulin molecule produced by B lymphoid cells with a specific amino acid sequence. In some embodiments, the antibodies described herein comprise or consist of an antibody binding fragment. In some embodiments, the antibody binding fragment comprises or consists of a Fab, Fab′, a F(ab)′2, a single-chain Fv(scFv), a Fv fragment, or a Fc sequence. In some embodiments, the antibody comprises a human IgG. Antibodies are in some embodiments evoked in humans or other animals by a specific antigen (immunogen, such as HA and NA). Antibodies are in some embodiments characterized by reacting specifically with the antigen in some demonstrable way, antibody and antigen each being defined in terms of the other. “Eliciting an antibody response” refers in some embodiments to the ability of an antigen or other molecule to induce the production of antibodies.

In some embodiments, “antigen” or “immunogen” refers to a compound, composition, or substance that stimulates the production of antibodies or a T-cell response in an animal, including compositions that are injected or absorbed into an animal. In some embodiments, an antigen reacts with the products of specific humoral or cellular immunity, including those induced by heterologous immunogens. In some embodiments of the disclosed compositions and methods, the antigen is an influenza HA protein, an influenza NA protein, or both. As used herein, an “immunogenic composition” is in some embodiments a vaccine comprising an antigen (such as a plurality of seVLPs having different influenza HA proteins).

“Immune response” refers in some embodiments to a response of a cell of the immune system, such as a B-cell, T-cell, macrophage or polymorphonucleocyte, to a stimulus such as an antigen or vaccine (such as an influenza A or B HA and/or NA protein). In some embodiments, an immune response comprises any cell of the body involved in a host defense response, comprising for example, an epithelial cell that secretes an interferon or a cytokine. An immune response comprises, but is not limited to, an innate immune response or inflammation. As used herein, a protective immune response refers to an immune response that protects a subject from infection (prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, antibody production and the like.

An “isolated” biological component (such as a nucleic acid, protein, VLP, or virus) has in some embodiments been substantially separated or purified away from other biological components (such as cell debris, or other proteins or nucleic acids). In some embodiments biological components that have been “isolated” include those components purified by standard purification methods. The term also in some embodiments embraces recombinant nucleic acids, proteins, viruses and VLPs, as well as chemically synthesized nucleic acids or peptides.

In some embodiments, the term “purified” does not require absolute purity; rather, it is intended as a relative term. In some embodiments, a purified protein, virus, VLP or other compound is one that is isolated in whole or in part from naturally associated proteins and other contaminants. In some embodiments, the term “substantially purified” refers to a protein, virus, VLP or other active compound that has been isolated from a cell, cell culture medium, or other crude preparation and subjected to fractionation to remove various components of the initial preparation, such as proteins, cellular debris, and other components. In some embodiments, an isolated or purified biological component, protein, virus, VLP or other compound has or comprises 1%, 0.75%, 0.5%, 0.25%, 0.1%, 0.05%, 0.01%, 0.005%, 0.001%, 0.0005%, 0.0001%, 0.00005%, 0.00001%, 0.000005%, or 0.000001%, or a range of percentages defined by any two of the aforementioned percentages, contaminants. In some embodiments, an isolated or purified biological component, protein, virus, VLP or other compound has or comprises less than 1%, 0.75%, 0.5%, 0.25%, 0.1%, 0.05%, 0.01%, 0.005%, 0.001%, 0.0005%, 0.0001%, 0.00005%, 0.00001%, 0.000005%, or 0.000001% contaminants.

In some embodiments, “lipids” include naturally occurring, semisynthetic and totally synthetic lipids. Some examples of lipids used to produce VLPs include DOPC, DOPE, DSPE (1,2-Distearoyl-sn-glycero-3-phosphoethanolamine) and DSPE-PEG (1,2-distearoyl-sn-glycero-3-phosphoethanolamine-N-[amino(polyethylene glycol)-2000] (ammonium salt)), cholesterol, and their derivatives. Some embodiments include a mixture such as one comprising phosphatidyl choline (50 mg/ml), cholesterol (20 mg/ml), phosphatidyl ethanolamine (10 mg/ml), phosphatidyl serine (10 mg/ml), sphingomyelin (20 mg/ml) and phosphatidyl inositol (2.5 mg/ml) mixed in a ratio of 10:4.25:3:1:3.

In some embodiments, a first nucleic acid sequence is “operably linked” with a second nucleic acid sequence when the first nucleic acid sequence is placed in a functional relationship with the second nucleic acid sequence. In some embodiments, a promoter is operably linked to a coding sequence if the promoter affects the transcription or expression of the coding sequence. In some embodiments, operably linked DNA sequences are contiguous and, where necessary to join two protein-coding regions, in the same reading frame.

The similarity between amino acid or nucleic acid sequences is in some cases expressed in terms of the similarity between the sequences, otherwise referred to as “sequence identity.” In some embodiments, sequence identity is measured in terms of percentage identity (or similarity or homology); e.g. the higher the percentage, the more similar the two sequences are. In some embodiments, homologs or variants of a given gene or protein possess a relatively high degree of sequence identity when aligned using standard methods.

In some embodiments, “therapeutically effective amount” refers to a quantity of a specified agent sufficient to achieve a desired effect in a subject being treated with that agent. In some embodiments, this is an amount of a vaccine or VLP useful for eliciting an immune response in a subject and/or for preventing infection or disease caused by influenza virus. In some embodiments, a therapeutically effective amount of a vaccine is an amount sufficient to increase resistance to, prevent, ameliorate, and/or treat infection caused by influenza virus (such as influenza A, influenza B, or both) in a subject without causing a substantial cytotoxic effect in the subject. In some embodiments, the effective amount of a vaccine useful for increasing resistance to, preventing, ameliorating, and/or treating infection in a subject will be dependent on, for example, the subject being treated, the manner of administration of the therapeutic composition and other factors such as adjuvants.

In some embodiments, a “vaccine” refers to or comprises a preparation of immunogenic material capable of stimulating an immune response, administered for the prevention, amelioration, or treatment of disease, such as an infectious disease. In some embodiments, the immunogenic material is a VLP disclosed herein. In some embodiments, vaccines elicit both prophylactic (preventative) and therapeutic responses. In some embodiments, methods of administration vary according to the vaccine, or include inoculation, ingestion, intranasal, intradermal, or other forms of administration. In some embodiments, vaccines are administered with an adjuvant to enhance the immune response.

In some embodiments, a VLP refers to or comprises an enveloped structure resembling a virus made up of one of more viral structural proteins, but which lacks a viral genome. In some embodiments, VLPs lack a viral genome and are non-infectious. In some embodiments, VLPs are divided into non-enveloped and eVLPs. In some embodiments, enveloped VLPs include a lipid membrane. In some embodiments, the VLP presents a properly folded, functional antigen. In some embodiments, the VLPs present HA that binds to receptors on epithelial cells or red blood cells. In some embodiments, the VLPs present NA and have enzymatic activity that cleaves sialic acids. In some embodiments, the VLPs comprise synthetic enveloped VLPs (seVLPs). In some embodiments, the seVLPs presents or comprise HA or NA proteins, and include a viral core protein that drives budding and release of particles from a host cell (such as influenza M1, M2 or both). In some embodiments, the VLPs comprise smVLPs. In some embodiments, the smVLPs comprise a nanodisc. In some embodiments, the nanodisc comprises a synthetic, semisynthetic or natural lipid bilayer comprising a first side and a second side; an anchor molecule embedded in the lipid bilayer; and an antigen bound to the anchor molecule.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. In some embodiments, the term “a sample” comprises a plurality of samples, comprising mixtures thereof

In some embodiments, a “subject” is a biological entity containing expressed genetic materials. In some embodiments, the subject is a mammal. In some embodiments, the mammal is a human.

As used herein, the term “about” a number refers to that number plus or minus 10% of that number. The term “about” a range refers to that range minus 10% of its lowest value and plus 10% of its greatest value.

As used herein, the terms “treatment” or “treating” are, in some embodiments, used in reference to a pharmaceutical regimen for obtaining beneficial or desired results in the recipient. Beneficial or desired results include but are not limited to a therapeutic benefit and/or a prophylactic benefit. In some embodiments, a therapeutic benefit refers to eradication or amelioration of symptoms or of an underlying disorder being treated. In some embodiments, a therapeutic benefit is achieved with the eradication or amelioration of one or more of the physiological symptoms associated with the underlying disorder such that an improvement is observed in the subject, notwithstanding that the subject may still be afflicted with the underlying disorder. In some embodiments, a prophylactic effect comprises delaying, preventing, or eliminating the appearance of a disease or condition, delaying or eliminating the onset of symptoms of a disease or condition, slowing, halting, or reversing the progression of a disease or condition, or any combination thereof. In some embodiments, for prophylactic benefit, a subject at risk of developing a particular disease, or to a subject reporting one or more of the physiological symptoms of a disease undergoes treatment, even if a diagnosis of the disease has not been made. In some embodiments, a therapeutic benefit comprises immunization against a disease.

The section headings used herein are for organizational purposes only and are not to be construed as limiting the subject matter described.

### III. SEVLPS AND SMVLPS

Disclosed herein, in certain embodiments, are synthetic enveloped VLPs (seVLPs) comprising or consisting of (a) a synthetic lipid vesicle comprising a lipid bilayer comprising an inner surface and an outer surface; (b) an anchor molecule embedded in the lipid bilayer; and (c) an antigen bound to the anchor molecule. Also disclosed herein, in certain embodiments, are smVLPs comprising a nanodisc comprising a synthetic, semisynthetic or natural lipid bilayer comprising a first side and a second side; an anchor molecule embedded in the lipid bilayer; and an antigen bound to the anchor molecule. In some embodiments, the VLPS are stable at room temperature. In some embodiments, the lipid bilayer is synthetic. In some embodiments, the lipid bilayer is semi-synthetic. In some embodiments, the lipid bilayer is natural or non-synthetic. In some embodiments, the lipid bilayer comprises synthetic lipids. In some embodiments, the lipid bilayer is semi-synthetic, and comprises natural or non-synthetic lipids, and synthetic lipids. In some embodiments, the lipid bilayer comprises natural lipids.

In some embodiments, the antigen is made using purified recombinant proteins. In some embodiments, the recombinant proteins are produced from cultured cells. In some embodiments, the cultured cells comprise a nucleic acid encoding an antigen.

In some embodiments, the VLPs (e.g. seVLPs or smVLPs) comprise defined purified recombinant proteins mixed with defined lipids. In some embodiments, the VLPs comprise or consist of a chemically defined fully synthetic seVLPs. In some embodiments, the seVLPs contain the antigen proteins are embedded in the membrane. In some embodiments, the seVLPs contain the antigen proteins comprising an anchor molecule as described herein that is embedded in the membrane. In some embodiments, seVLPs comprise the antigen proteins embedded in the membrane by virtue of a membrane anchor domain while the surface of the seVLP is decorated with the hydrophilic domains of an antigenic protein of interest. In some embodiments, a vaccine formulation comprises combination of antigens in a single seVLP. In some embodiments, different seVLPs are mixed together into a single vaccine.

In some embodiments, the seVLPs comprise antigens anchored in place by a protein lipophilic transmembrane domain of the antigen whereas hydrophilic domains of the antigen are displayed both on the inner and outer surface of the lipid membrane. In some embodiments, the lipids of the membrane serve to enhance the immune response and to present the antigens is a structured ordered array to also enhance the immune response. In some embodiments, the antigen retains its native three-dimensional conformation within the seVLP or liposome.

In some embodiments, the VLPs comprise or consist of smVLPs. In some embodiments, the smVLP comprises a disc. In some embodiments, the disc is a nanodisc. In some embodiments, the nanodisc comprises a membrane. In some embodiments, the nanodisc or membrane comprises a synthetic, semisynthetic or natural lipid bilayer. In some embodiments, lipids of the lipid bilayer comprise a hydrophobic aliphatic side chain. In some embodiments, lipids of the lipid bilayer comprise a hydrophilic head. In some embodiments, the nanodisc comprises a first side and a second side. In some embodiments, each of the first and/or second side is flat. In some embodiments, each of the first and/or second side comprises an antigen embedded in the lipid bilayer. In some embodiments, the nanodisc comprises an edge. In some embodiments, the edge is circular. In some embodiments, the edge comprises a perimeter. In some embodiments, the nanodisc is toroidal, discoidal, or coin shaped.

In some embodiments, the nanodisc is made from or comprises polymethacrylate (PMA) copolymers. In some embodiments, the PMA copolymers are amphiphilic. In some embodiments, the PMA copolymers are toroidal. In some embodiments, the PMA copolymers wrap around a perimeter or edge of the nanodisc. In some embodiments, the PMA copolymers form a toroidal shape around the perimeter or edge of the nanodisc. In some embodiments, the nanodisc is made from or comprises styrene-maleic acid lipid particles (SMALPs). In some embodiments, the SMALPs are toroidal. In some embodiments, the SMALPs are amphiphilic. In some embodiments, the SMALPs wrap around a perimeter or edge of the nanodisc. In some embodiments, the SMALPs form a toroidal shape around the perimeter or edge of the nanodisc. In some embodiments, the SMALPs comprise SMALP 25010P, SMALP 30010P, and/or SMALP 40005P (e.g. from Polyscience, Geleen, Netherlands). In some embodiments, the nanodisc comprises PMA copolymers and SMALPs. In some embodiments, the nanodisc does not comprise SMALPS. In some embodiments, the nanodisc does not comprise PMA copolymers. In some embodiments, the nanodisc does not comprise a membrane scaffold protein (MSPS) or an amphipathic MSPS. In some embodiments, the nanodisc does not comprise apolipoprotein A-1 (ApoA). In some embodiments, the nanodisc comprises a non-immunogenic 22 amino acid mimetic peptides derived from the repeat alpha helix domain of ApoA. In some embodiments, the nanodisc is formulated for human use. In some embodiments the PMA copolymer provides a benefit of making the nanodisc suitable for human use. In some embodiments, the PMA is nontoxic. In some embodiments the SMALPs provide a benefit of making the nanodisc suitable for human use. In some embodiments, the SMALPs are nontoxic. In some embodiments, the nanodisc comprises a polymethacrylate copolymer (e.g. N—C4-52-6.9). In some embodiments, SMA is unstable at a low pH or in the presence of divalent metal ions.

In some embodiments, the nanodisc comprises DIBMA. In some embodiments, the nanodisc comprises a DIBMAA co-polymer. In some embodiments, the DIBMA co-polymer is toroidal. In some embodiments, the nanodisc comprises an amphiphilic toroidal DIBMA co-polymer. In some embodiments, the smVLP is styrene-free, or comprises a styrene-free polymer. In some embodiments, the smVLP comprises a DIBMA or polymethacrylate copolymer (PMA). In some embodiments, the DIBMA or PMA form nanodiscs and affect lipid acyl chains or have improved stability towards divalent metal ions compared to SMA.

In some embodiments, the nanodisc membrane comprises one or more membrane bound antigen proteins. In some embodiments, the nanodisc comprises 5, 10, 25, 50, 75, 100, 125, 150, 175, 200, 225, 250, 300, 400, or 500 nM diameter, or a range of diameters defined by any two of the aforementioned diameters. In some embodiments, the nanodisc comprises a 5-200 nM diameter. In some embodiments, the nanodisc comprises a 50-200 nM diameter. In some embodiments, the nanodisc comprises diameter less than 5, 10, 25, 50, 75, 100, 125, 150, 175, 200, 225, 250, 300, 400, or 500 nM. In some embodiments, the nanodisc comprises diameter greater than 5, 10, 25, 50, 75, 100, 125, 150, 175, 200, 225, 250, 300, 400, or 500 nM. In some embodiments, the nanodisc comprises diameter of less than 50 nM. In some embodiments, the nanodisc comprises diameter of greater than 50 nM. In some embodiments, the nanodisc comprises a 50-100 nM diameter. In some embodiments, the nanodisc comprises a 100-150 nM diameter. In some embodiments, the nanodisc comprises a 150-200 nM diameter. In some embodiments, the nanodisc comprises a 75-125 nM diameter. In some embodiments, the diameter is a diameter of a lipid bilayer of the VLP. In some embodiments, the diameter is a diameter of a toroidal protein on an outside edge of the VLP.

In some embodiments, the nanodisc comprises a diameter larger than 50 nM with an antigen (e.g. an influenza HA antigen) embedded in a lipid membrane of the nanodisc. In some embodiments, the nanodisc does not comprise an envelope or lipid envelope. In some embodiments, the nanodisc comprises a single antigen or anchor molecule. In some embodiments, the smVLP comprises a large nanodisc. In some embodiments, the nanodisc comprises multiple antigens and/or anchor molecules. In some embodiments, the nanodisc or large nanodisc embeds an array of antigens. In some embodiments, the nanodisc is a component of a vaccine comprising multiple smVLPs or polyvalent smVLPs. In some embodiments, first side of the lipid bilayer comprises a first anchor molecule and/or a first antigen, and the second side of the lipid bilayer comprises a second anchor molecule and/or a second antigen.

In some embodiments, the nanodisc comprises an anchor molecule embedded in the lipid bilayer, and an antigen bound to the anchor molecule. In some embodiments, the antigen is embedded directly in the lipid bilayer.

A. Lipid Vesicles

In some embodiments, the lipid vesicle comprises a first lipid such as a phosphatidylcholine species. In some embodiments, the lipid vesicle comprises a second lipid such as a phosphatidylethanolamine species. In some embodiments, the lipid vesicle comprises the first lipid and the second lipid at a predetermined ratio. In some embodiments, the predetermined ratio is between 1:0.25 and 1:4. In some embodiments, the lipid vesicle comprises the first lipid and the second lipid at a predetermined ratio between 1:0.25 and 1:4. In some embodiments, the lipid vesicle is part of an seVLP as described herein. Some embodiments include a VLP with a first lipid, a second lipid, and/or a third lipid as described herein. In some embodiments, the lipid or lipids of a smVLP do not form a lipid vesicle. In some embodiments, a smVLP does not comprise a lipid vesicle.

In some embodiments, the first lipid and/or the second lipid each comprise an acyl chain comprising 1, 2, 3, 4, 5, 6, 7, 8, 9, 10, 11, 12, 13, 14, 15, 16, 17, 18, 19, 20, or more carbon atoms, or a range of carbon atoms defined by any two of the aforementioned numbers. In some embodiments, the first lipid and/or the second lipid each comprise an acyl chain comprising between 4 and 18 carbon atoms. In some embodiments, the first lipid and/or the second lipid each comprise four or less unsaturated bonds. In some embodiments, the first lipid comprises 1, 2, 3, 4, 5, 6, 7, 8, 9, 10, or less unsaturated bonds, or a range of unsaturated bond defined by any two of the aforementioned numbers. In some embodiments, the second lipid comprises 1, 2, 3, 4, 5, 6, 7, 8, 9, 10, or less unsaturated bonds, or a range of unsaturated bond defined by any two of the aforementioned numbers.

In some embodiments, the first lipid and/or the second lipid of the lipid vesicle comprise or consist of a purified lipid. In some embodiments, the purified lipid is at least 75.0%, 80.0%, 85.0%, 90.0%, 91.0%, 92.0%, 93.0%, 94.0%, 95.0%, 96.0%, 97.0%, 97.5%, 98.0%, 98.5%, 99.0%, 99.5%, 99.9%, 100%, or a range of percentages defined by any two of the aforementioned percentages, pure. In some embodiments, the purified lipid is at least 99% pure.

In some embodiments, the first lipid comprises 1,2-Dioleoyl-sn-glycero-3-phosphocholine (DOPC). In some embodiments, the second lipid comprises 1,2-Dioleoyl-sn-glycero-3-phosphoethanolamine (DOPE). In some embodiments, the vaccine comprises one or more lipids such as DOPC or DOPE. In some embodiments, the vaccine comprises cholesterol. In some embodiments, the vaccine comprises DSPE-peg2000 (1,2 distearoyl-sn-glycero-3-phophoethanoamine-N[amino(polyethelene glycol)-2000] (ammonium salt), or a related lipid.

In some embodiments, the lipid vesicle comprises a sterol or sterol derivative. In some embodiments, the sterol or sterol derivative comprises cholesterol or DC-cholesterol. In some embodiments, the lipid vesicle comprises the sterol or sterol derivative at a ratio of 0, 5, 10, 15, 20, 25, 30, 35, 40, 45, or 50 mol %, or a range defined by any two of the aforementioned mole mole percentages, in relation to the first lipid and/or the second lipid. In some embodiments, the lipid vesicle comprises the sterol or sterol derivative at a ratio of 0-30 mol % in relation to the first lipid and/or the second lipid.

In some embodiments, the lipid vesicle, the first lipid of the lipid vesicle, and/or the second lipid of the lipid vesicle are synthetic. In some embodiments, the lipid vesicle, the first lipid of the lipid vesicle, and/or the second lipid of the lipid vesicle are natural lipids. In some embodiments, the lipid vesicle, the first lipid of the lipid vesicle, and/or the second lipid of the lipid vesicle comprise natural and synthetic lipids. In some embodiments, the lipid vesicle, the first lipid of the lipid vesicle, and/or the second lipid of the lipid vesicle are free or substantially free of biologic material.

B. Antigens

In some embodiments, the lipid vesicle comprises an outward surface, and wherein the antigen is presented on the outward surface of the lipid vesicle. In some embodiments, the lipid vesicle comprises an inward surface, and wherein the antigen is presented on the inward surface of the lipid vesicle.

In some embodiments, the antigen is produced in bacteria, yeast, plants, insect cells or mammalian cells. In some embodiments, the antigen is, consists of, or comprises a purified antigen. In some embodiments, the purified antigen is at least 75.0%, 80.0%, 85.0%, 90.0%, 91.0%, 92.0%, 93.0%, 94.0%, 95.0%, 96.0%, 97.0%, 97.5%, 98.0%, 98.5%, 99.0%, 99.5%, 99.9%, 100%, or a range of percentages defined by any two of the aforementioned percentages, pure. In some embodiments, the purified antigen is at least 99% pure. In some embodiments, the antigen is purified before being mixed with one or more lipids.

In some embodiments, the antigen is bound directly to a membrane anchor as described herein. In some embodiments, the antigen comprises the membrane anchor.

In some embodiments, the antigen comprises a tag such as a hexahistidine tag or a flag tag.

In some embodiments, the VLPs (e.g. seVLPs or smVLPs) comprise a transmembrane antigen such as respiratory syncytial virus, chickenpox, HIV, SARS, Ebola, Nipah, Dengue, Rift Valley fever, rabies, measles, mumps, rubella, Lassa and Marburg viruses. The synthetic nature of some embodiments combines defined lipids with defined proteins and teaches techniques that extend in some instances to any antigen of interest. In some embodiments, the VLP includes a coronavirus antigen, such as a coronavirus antigen described herein.

In some embodiments, the antigen is a pathogen antigen. In some embodiments, the antigen is a protein or component of a pathogen. In some embodiments, the pathogen is a virus or a parasite. Non-limiting examples of types of viruses and parasites a VLP targets in some embodiments include a lentivirus, a flavivirus, a filovirus, a coronavirus, a paramyxovirus, a HPV, a herpes virus, a hepatitis C (HepC) virus, a plasmodium parasite, or a trypanosoma parasite.

In some embodiments, the antigen is a cancer-associated peptide or antigen, or a fragment thereof. Examples of cancer-associated antigens include, but are not limited to, tumor-specific immunoglobulin variable regions, GM2, Tn, sTn, TF, Globo H, Le(y), MUC1, MUC2, MUC3, MUCO, MUC5AC, MUC5B, MUC7, carcinoembryonic antigens, beta chain of human chorionic gonadotropin (hCG beta), C35, HER2/neu, CD20, PSMA, EGFRvIII, KSA, PSA, PSCA, GP100, MAGE 1, MAGE 2, TRP 1, TRP 2, tyrosinase, MART-1, PAP, CEA, BAGE, MAGE, RAGE, and related proteins.

In some embodiments, the antigen is a bacterial peptide or antigen, or a fragment thereof. Examples of bacterial antigens include, but are not limited to, Actinomyces antigens, Bacillus antigens, e.g., immunogenic antigens from Bacillus anthracis, Bacteroides antigens, Bordetella antigens, Bartonella antigens, Borrelia antigens, e.g., B. burgdorferi OspA, Brucella antigens, Campylobacter antigens, Capnocytophaga antigens, Chlamydia antigens, Clostridium antigens, Corynebacterium antigens, Coxiella antigens, Dermatophilus antigens, Enterococcus antigens, Ehrlichia antigens, Escherichia antigens, Francisella antigens, Fusobacterium antigens, Haemobartonella antigens, Haemophilus antigens, e.g., H. influenzae type b outer membrane protein, Helicobacter antigens, Klebsiella antigens, L form bacteria antigens, Leptospira antigens, Listeria antigens, Mycobacteria antigens, Mycoplasma antigens, Neisseria antigens, Neorickettsia antigens, Nocardia antigens, Pasteurella antigens, Peptococcus antigens, Peptostreptococcus antigens, Pneumococcus antigens, Proteus antigens, Pseudomonas antigens, Rickettsia antigens, Rochalimaea antigens, Salmonella antigens, Shigella antigens, Staphylococcus antigens, Streptococcus antigens, e.g., S. pyogenes M proteins, Treponema antigens, and Yersinia antigens, e.g., Y. pestis F1 and V antigens.

In some embodiments, the antigen is a fungal peptide or antigen, or a fragment thereof. Examples of parasitic antigens include, but are not limited to Balantidium coli antigens, Entamoeba histolytica antigens, Fasciola hepatica antigens, Giardia lamblia antigens, Leishmania antigens, and Plasmodium antigens (e.g., Plasmodium falciparum antigens).

In some embodiments, the antigen is a parasitic peptide or antigen, or a fragment thereof. Examples of parasitic include, but are not limited to Balantidium coli antigens, Entamoeba histolytica antigens, Fasciola hepatica antigens, Giardia lamblia antigens, Leishmania antigens, and Plasmodium antigens (e.g., Plasmodium falciparum antigens).

In some embodiments, the antigen is a viral peptide or antigen, or a fragment thereof. Examples of viral antigenic and immunogenic antigens include, but are not limited to, adenovirus antigens, alphavirus antigens, calicivirus antigens, e.g., a calicivirus capsid antigen, coronavirus antigens, distemper virus antigens, Ebola virus antigens, enterovirus antigens, flavivirus antigens, hepatitis virus (A-E) antigens, e.g., a hepatitis B core or surface antigen, herpesvirus antigens, e.g., a herpes simplex virus or varicella zoster virus glycoprotein, immunodeficiency virus antigens, e.g., the human immunodeficiency virus envelope or protease, infectious peritonitis virus antigens, influenza virus antigens, e.g., an influenza A hemagglutinin, neuraminidase, or nucleoprotein, leukemia virus antigens, Marburg virus antigens, orthomyxovirus antigens, papilloma virus antigens, parainfluenza virus antigens, e.g., the hemagglutinin/neuraminidase, paramyxovirus antigens, parvovirus antigens, pestivirus antigens, picorna virus antigens, e.g., a poliovirus capsid polypeptide, pox virus antigens, e.g., a vaccinia virus polypeptide, rabies virus antigens, e.g., a rabies virus glycoprotein G, reovirus antigens, retrovirus antigens, and rotavirus antigens.

In the case of a lentivirus, the antigen is in some embodiments a HIV antigen or protein. In the case of a flavivirus, the antigen is in some embodiments a Dengue virus, a Zika virus, or a West Nile virus antigen or protein. In the case of a Filovirus, the antigen is in some embodiments an Ebola virus, a Marburg virus, or a Ravies virus antigen or protein. In the case of a coronavirus, the antigen is in some embodiments a MERS virus or a SARS virus antigen or protein. In the case of a paramyxovirus, the antigen is in some embodiments a Respiratory Syncytial Virus (RSV) or a Nipah virus antigen or protein. In the case of a plasmodium parasite, the antigen is in some embodiments a malaria parasite antigen or protein. In the case of a trypanosoma parasite, the antigen is in some embodiments a Chagas parasite, a Sleeping Sickness parasite, or a Leishmaniasis parasite antigen or protein.

Some non-limiting examples of suitable antigens include glycoproteins such as the surface proteins and glycoproteins (GPs) of an enveloped virus such as the Gag and/or Env of HIV, the HA, and/or NA and/or M2 proteins of influenza, the C, E3, E2, 6k, and/or E1 proteins of Chikungunya, the S, E, M and/or N proteins of SARS, the M, G, F proteins of Nipah, the V40, GP, NP proteins of Ebola, the prM and E proteins of Dengue, the Gn, Gc, or NP proteins of Rift Valley fever virus or the GPC, NP or Z proteins of Lassa virus.

In some embodiments, the antigen comprises a hybrid protein that contains or comprises a membrane anchor such as a membrane anchor domain fused to a non-membrane protein such as the L2 protein of HPV fused to the membrane anchor domain of the influenza HA. In some embodiments, antigens are or include any number of tumor related antigens such as MUC, HPV E6 and/or E7, MAGE-A3, or CEA.

In some embodiments, the antigen comprises a glycoprotein of any enveloped virus. In some embodiments, the antigen adheres to the outside surface of a lipid containing structure forming a seVLP as described herein. In some embodiments, the antigen adheres a side of a smVLP.

In some embodiments, the antigen comprises a protein fusion. In some embodiments, the antigen is fused to a membrane anchor domain.

In some embodiments, the antigen comprises a carbohydrate antigen chemically attached to a carrier protein that contains a membrane anchor. In some embodiments, the antigen is without a membrane anchor.

In some embodiments, the antigen comprises a fusion protein. In some embodiments of the fusion protein, the antigen is fused to the transmembrane domain of surface protein or surface glycoprotein. For example, a HPV is used in some embodiments. HPV infection is a precursor to some cervical cancers. Some HPV VLPs are based on the immunodominant protein L1, the outer capsid protein, but L1 based HPV VLPs are strain specific. Gardasil 9® (Merck) is composed of nine different L1 proteins that assemble into non-enveloped VLPs. In contrast the L2 protein is in some embodiments poorly immunogenic but is a common antigen for HPV strains. In some embodiments, to make an VLP based on the L2 protein of HPV, L2 is fused to the transmembrane domain of the influenza HA. In some embodiments, this is where the antigen is at the N-terminus and the HA transmembrane domain is at the C-terminus of the protein. In some embodiments, a VLP would yield a structured and patterned array of the normally poorly immunogenic L2 protein of HPV. In some embodiments, an HPV VLP based on L2 would be expected to protect against other HPV strains. In some embodiments, fusion antigens of the E6 and E7 proteins of HPV are used to create VLPs that treat patients with cervical cancer.

1. Influenza Antigens

In some embodiments, the antigen is an influenza virus antigen, or a variant or fragment thereof. Influenza virus is a segmented negative-strand RNA virus included in the Orthomyxoviridae family. There are three types of Influenza viruses, A, B and C. Influenza A virus (IAV): A negative-sense, single-stranded, segmented RNA virus, which has eight RNA segments (PB2, PB1, PA, NP, M, NS, HA and NA) that code for 11 proteins, comprising RNA-directed RNA polymerase proteins (PB2, PB1 and PA), nucleoprotein (NP), neuraminidase (NA), hemagglutinin (subunits HA1 and HA2), the matrix proteins (M1 and M2) and the non-structural proteins (NS1 and NS2). This virus is prone to rapid evolution by error-protein polymerase and by segment reassortment. The host range of influenza A is quite diverse, and comprises humans, birds (e.g., chickens and aquatic birds), horses, marine mammals, pigs, bats, mice, ferrets, cats, tigers, leopards, and dogs. In animals, most influenza A viruses cause mild localized infections of the respiratory and intestinal tract. In some embodiments, highly pathogenic influenza A strains, such as H5N1, cause systemic infections in poultry in which mortality reaches 100%. In some embodiments, animals infected with influenza A act as a reservoir for the influenza viruses and certain subtypes cross the species barrier to humans.

In some embodiments, the antigen is an influenza A virus antigen, or a variant or fragment thereof. Influenza A viruses are classified into subtypes based on allelic variations in antigenic regions of two genes that encode surface glycoproteins, namely, hemagglutinin (HA) and neuraminidase (NA) which are required for viral attachment and cellular release. There are currently 18 different influenza A virus HA antigenic subtypes (H1 to H18) and 11 different influenza A virus NA antigenic subtypes (N1 to N11). In some embodiments, 1-H16 and N1-N9 are found in wild bird hosts and are a pandemic threat to humans. H17-H18 and N10-N11 have been described in bat hosts and are not currently thought to be a pandemic threat to humans.

Specific examples of influenza A include, but are not limited to: H1N1 (such as 1918 H1N1), H1N2, H1N7, H2N2 (such as 1957 H2N2), H2N1, H3N1, H3N2, H3N8, H4N8, H5N1, H5N2, H5N8, H5N9, H6N1, H6N2, H6N5, H7N1, H7N2, H7N3, H7N4, H7N7, H7N9, H8N4, H9N2, H1ON1, H1ON7, H1ON8, H11N1, H11N6, H12N5, H13N6, and H14N5. In one example, influenza A comprises those known to circulate in humans such as H1N1, H1N2, H3N2, H7N9, and H5N1.

In animals, some influenza A viruses cause self-limited localized infections of the respiratory tract in mammals and/or the intestinal tract in birds. In some embodiments, highly pathogenic influenza A strains, such as H5N1, cause systemic infections in poultry in which mortality reaches 100%. In 2009, H1N1 influenza is the most common cause of human influenza. A new strain of swine-origin H1N1 emerged in 2009 and is declared pandemic by the World Health Organization. This strain is referred to as “swine flu.” H1N1 influenza A viruses were also responsible for the Spanish flu pandemic in 1918, the Fort Dix outbreak in 1976, and the Russian flu epidemic in 1977-1978.

In some embodiments, the antigen comprises an influenza B virus antigen, or a variant or fragment thereof. Influenza B virus (IBV) is a negative-sense, single-stranded, RNA virus, which has eight RNA segments. The capsid of IBV is enveloped while its virion comprises an envelope, matrix protein, nucleoprotein complex, a nucleocapsid, and a polymerase complex. The surface projection are made of neuraminidase (NA) and hemagglutinin. This virus is less prone to evolution than influenza A, but it mutates enough such that lasting immunity has not been achieved. The host range of influenza B is narrower than influenza A, and is only known to infect humans and seals. Influenza B viruses are not divided into subtypes, but are further broken down into lineages and strains. Specific examples of influenza B include, but are not limited to: B/Yamagata, B/Victoria, B/Shanghai/361/2002 and B/Hong Kong/330/2001.

In some embodiments, the antigen is an influenza virus antigen or protein, or a fragment thereof. In some embodiments, the influenza protein is a HA, NA, M1, M2, NS1, NS2, PA, PB1, or PB2 influenza protein, or a fragment thereof

In some embodiments, the influenza protein comprises an amino acid sequence that is 75.0%, 80.0%, 85.0%, 90.0%, 91.0%, 92.0%, 93.0%, 94.0%, 95.0%, 96.0%, 97.0%, 97.5%, 98.0%, 98.5%, 99.0%, 99.5%, 99.9%, 100%, or a range of percentages defined by any two of the aforementioned percentages, identical to any of SEQ ID NOs: 1-14, or a fragment thereof. In some embodiments, the influenza protein comprises an amino acid sequence that is 75.0%, 80.0%, 85.0%, 90.0%, 91.0%, 92.0%, 93.0%, 94.0%, 95.0%, 96.0%, 97.0%, 97.5%, 98.0%, 98.5%, 99.0%, 99.5%, 99.9%, 100%, or a range of percentages defined by any two of the aforementioned percentages, identical to SEQ ID NO: 15 or 16, or a fragment thereof. In some embodiments, the influenza protein comprises an amino acid sequence that has no more than 1, 2, 3, 4, 5, 6, 7, 8, 9, 10, 20, 40, or a range defined by any of the aforementioned integers, amino acid substitutions, deletions, and/or insertions, compared to any of SEQ ID NOs: 1-14, or a fragment thereof. In some embodiments, the influenza protein comprises an amino acid sequence that has no more than 1, 2, 3, 4, 5, 6, 7, 8, 9, 10, 20, 40, or a range defined by any of the aforementioned integers, amino acid substitutions, deletions, and/or insertions, compared to SEQ ID NO: 15 or 16, or a fragment thereof. In some embodiments, the antigen comprises an amino acid sequence in accordance with SEQ ID NO: 15, or a variant thereof. In some embodiments, the antigen comprises an amino acid sequence in accordance with SEQ ID NO: 16, or a variant thereof

In some embodiments, the influenza protein is encoded by a nucleic acid with a sequence that is 75.0%, 80.0%, 85.0%, 90.0%, 91.0%, 92.0%, 93.0%, 94.0%, 95.0%, 96.0%, 97.0%, 97.5%, 98.0%, 98.5%, 99.0%, 99.5%, 99.9%, 100%, or a range of percentages defined by any two of the aforementioned percentages, identical to a nucleic acid sequence encoding any of amino acid SEQ ID NOs: 1-14, or a fragment thereof. In some embodiments, the influenza protein is encoded by a nucleic acid with a sequence that is 75.0%, 80.0%, 85.0%, 90.0%, 91.0%, 92.0%, 93.0%, 94.0%, 95.0%, 96.0%, 97.0%, 97.5%, 98.0%, 98.5%, 99.0%, 99.5%, 99.9%, 100%, or a range of percentages defined by any two of the aforementioned percentages, identical to a nucleic acid sequence encoding amino acid SEQ ID NO: 15 or 16, or a fragment thereof. In some embodiments, the influenza protein is encoded by a nucleic acid with a sequence that has no more than 1, 2, 3, 4, 5, 6, 7, 8, 9, 10, 20, 40, or a range defined by any of the aforementioned integers, nucleic acid substitutions, deletions, and/or insertions, compared to a nucleic acid sequence encoding any of amino acid SEQ ID NOs: 1-14, or a fragment thereof. In some embodiments, the influenza protein is encoded by a nucleic acid with a sequence that has no more than 1, 2, 3, 4, 5, 6, 7, 8, 9, 10, 20, 40, or a range defined by any of the aforementioned integers, nucleic acid substitutions, deletions, and/or insertions, compared to a nucleic acid sequence encoding amino acid SEQ ID NO: 15 or 16, or a fragment thereof

In some embodiments, the influenza virus is of type A type B, type C, or type D. In some embodiments, if a virus is a type A flu virus, it is H1N1, H1N2, H3N1, H3N2, or H2N3. In some embodiments, the flu virus is H2N2, H5N1, or H7N9.

Examples of flu virus strains are listed in Table 1. Some VLPs comprise a set of antigens that activate an immune response in a subject to at least 80% of strains in Table 1. In some embodiments, the VLP comprises a set of antigens that activate an immune response in a subject to at least 50%, 55%, 60%, 65%, 70%, 75%, 80%, 85%, at least 90%, at least 95%, at least 99%, or at least 100% of strains in Table 1.

In some embodiments, the VLP comprises an antigen of a strain in Table 1. Some VLPs include one or more homologues of one or more antigens of strains in Table 1. In some cases, such a homologue comprises at least 90% sequence identity to an antigen in Table 1. In some cases, such a homologue comprises at least 80% sequence identity to an antigen in Table 1. In some cases, such a homologue comprises at least 85% sequence identity to an antigen in Table 1. In some cases, such a homologue comprises at least 95% sequence identity to an antigen in Table 1. In some cases, such a homologue comprises at least 99% sequence identity to an antigen in Table 1.

TABLE 1
Examples of Flu Virus Strains

| | |
|---|---|
| H1N1 | A/Albany/12/1951 |
| | A/Beijing/22808/2009 |
| | A/Beijing/262/1995 |
| | A/Brevig Mission/1/1918 |
| | A/Brisbane/59/2007 |
| | A/California/04/2009 |
| | A/California/06/2009 |
| | A/California/07/2009 |
| | A/Chile/1/1983 |
| | A/England/195/2009 |
| | A/England/42/1972 |
| | A/New Caledonia/20/1999 |
| | A/New York/06/2009 |
| | A/New York/1/1918 |
| | A/New York/18/2009 |
| | A/New Jersey/8/1976 |
| | A/Ohio/07/2009 |
| | A/Ohio/UR06-0091/2007 |
| | A/Puerto Rico/8/1934 |
| | A/Puerto Rico/8/34/Mount Sinai |
| | A/Solomon Islands/3/2006 |
| | A/swine/Belgium/1/1998 |
| | A/Swine/Wisconsin/136/1997 |
| | A/Taiwan/01/1986 |
| | A/Texas/05/2009 |
| | A/Texas/36/1991 |
| | A/USSR/90/1977 |
| | A/USSR/92/1977 |
| | A/WSN/1933 |
| H1N2 | A/swine/Guangxi/13/2006 |
| H1N3 | A/duck/NZL/160/1976 |
| H2N2 | A/Ann Arbor/6/1960 |
| | A/Canada/720/2005 |
| | A/Guiyang/1/1957 |
| | A/Japan/305/1957 |
| H3N2 | A/Aichi/2/1968 |
| | A/Babol/36/2005 |
| | A/Brisbane/10/2007 |
| | A/California/7/2004 |
| | A/Chiang Rai/277/2011 |
| | A/Christchurch/4/1985 |
| | A/Fujian/411/2002 |
| | A/Guangdong-Luohu/1256/2009 |
| | A/Hong Kong/1/1968 |
| | A/Hong Kong/CUHK31987/2011 |
| | A/Indiana/07/2012 |
| | A/Memphis/1/68 |
| | A/Moscow/10/1999 |
| | A/New York/55/2004 |
| | A/Perth/16/2009 |
| | A/reassortant/IVR-155 |
| | A/Sydney/5/1997 |
| | A/Texas/50/2012 |
| | A/Victoria/208/2009 |
| | A/Victoria/210/2009 |
| | A/Victoria/3/1975 |
| | A/Victoria/361/2011 |
| | A/Wisconsin/15/2009 |
| | A/Wisconsin/67/X-161 /2005 |
| | A/Wyoming/03/2003 |
| | A/X-31 |
| H3N8 | A/canine/New York/145353/2008 |
| | A/equine/Gansu/7/2008 |
| H4N2 | A/duck/Hunan/8-19/2009 |
| H4N4 | A/mallard duck/Alberta/299/1977 |
| H4N6 | A/mallard/Ohio/657/2002 |
| | A/Swine/Ontario/01911-1/99 |
| H4N8 | A/chicken/Alabama/1/1975 |
| H5N1 | A/Anhui/1/2005 |
| | A/bar-headed goose/Qinghai/14/2008 |
| | A/bar-headed goose/Qinghai/1A/2005 |
| | A/barnswallow/Hong Kong/D10-1161/2010 |
| | A/Cambodia/R0405050/2007 |
| | A/Cambodia/S1211394/2008 |
| | A/chicken/Egypt/2253-1/2006 |
| | A/chicken/India/NIV33487/2006 |
| | A/chicken/Jilin/9/2004 |
| | A/chicken/VietNam/NCVD-016/2008 |
| | A/chicken/Yamaguchi/7/2004 |
| | A/Common magpie/Hong Kong/2256/2006 |
| | A/common magpie/Hong Kong/5052/2007 |
| | A/Duck/Hong Kong/p46/97 |
| | A/duck/Hunan/795/2002 |
| | A/duck/Laos/3295/2006 |
| | A/Egypt/2321-NAMRU3/2007 |
| | A/Egypt/3300-NAMRU3/2008 |
| | A/Egypt/N05056/2009 |
| | A/goose/Guangdong/1/96 |
| | A/goose/Guiyang/337/2006 |
| | A/Hong kong/213/03 |
| | A/Hong Kong/483/97 |
| | A/Hubei/1/2010 |
| | A/Hubei/2011 |
| | A/hubei/2011-CDC |
| | A/Indonesia/5/2005 |
| | A/Japanese white-eye/Hong Kong/1038/2006 |
| | A/Thailand/1(KAN-1)/2004 |
| | A/turkey/Turkey/1/2005 |
| | A/Vietnam/UT31413II/2008 |
| | A/whooper swan/Mongolia/244/2005 |
| | A/Xinjiang/1/2006 |
| H5N2 | A/American green-winged |
| | teal/California/HKWF609/07 |
| | A/ostrich/South Africa/AI1091/2006 |
| H5N3 | A/duck/Hokkaido/167/2007 |
| H5N8 | A/breeder duck/Korea/Gochang1/2014 |
| | A/broiler duck/Korea/Buan2/2014 |
| | A/duck/Jiangsu/k1203/2010 |
| | A/duck/NY/191255-59/2002 |
| | A/duck/Zhejiang/6D18/2013 |
| | A/duck/Zhejiang/W24/2013 |
| | A/turkey/Ireland/1378/1983 |
| H5N9 | A/chicken/Italy/22A/1998 |
| H6N1 | A/northern shoveler/California/HKWF115/2007 |
| H6N4 | A/chicken/HongKong/17/77 |
| H6N5 | A/shearwater/Australia/1/1973 |
| H6N6 | A/duck/Eastern China/11/2009 |
| H6N8 | A/mallard/Ohio/217/1998 |
| H7N1 | A/turkey/Italy/4602/99 |
| H7N2 | A/ruddy turnstone/New Jersey/563/2006 |
| H7N3 | A/chicken/SK/HR-00011/2007 |
| | A/turkey/Italy/214845/2002 |
| H7N7 | A/chicken/Netherlands/1/03 |
| | A/equine/Kentucky/1a/1975 |
| | A/Netherlands/219/2003 |
| H7N8 | A/mallard/Netherlands/33/2006 |
| H7N9 | A/Anhui/1/2013 |
| | A/Anhui/PA-1/2013 |
| | A/chicken/Zhejiang/DTID-ZJU01/2013 |
| | A/Hangzhou/1/2013 |
| | A/Hangzhou/3/2013 |
| | A/Huzhou/10/2013 |
| | A/Pigeon/Shanghai/S1069/2013 |
| | A/Shanghai/1/2013 |
| | A/Shanghai/4664T/2013 |
| | A/Shanghai/Patient3/2013 |
| | A/Zhejiang/1/2013 |
| | A/Zhejiang/DTID-ZJU10/2013 |
| H8N4 | A/pintail duck/Alberta/114/1979 |
| H9N2 | A/brambling/Beijing/16/2012 |
| | A/Chicken/Hong Kong/G9/1997 |
| | A/duck/Hong Kong/448/78 |
| | A/Guinea fowl/Hong Kong/WF10/99 |
| | A/Hong Kong/1073/99 |
| | A/Hong Kong/2108/2003 |
| | A/Hong Kong/3239/2008 |
| | A/Hong Kong/35820/2009 |
| H9N5 | A/shorebird/DE/261/2003 |
| H9N8 | A/chicken/Korea/164/04 |
| H10N3 | A/duck/Hong Kong/786/1979 |
| | A/duck/Hunan/S11205/2012 |
| | A/mallard/Minnesota/Sg-00194/2007 |
| H10N4 | A/mink/Sweden/3900/1984 |
| H10N7 | A/blue-winged teal/Louisiana/Sg-00073/2007 |
| H10N8 | A/duck/Guangdong/E1/2012 |
| | A/Jiangxi-Donghu/346/2013 |
| H10N9 | A/duck/Hong Kong/562/1979 |
| | A/duck/Hong Kong/562/1979 |
| H11N2 | A/duck/Yangzhou/906/2002 |
| | A/thick-billed murre/Newfoundland/031/2007 |
| H11N6 | A/duck/England/1/1956 |
| H11N9 | A/mallard/Alberta/294/1977 |
| H12N1 | A/mallard duck/Alberta/342/1983 |
| H12N3 | A/bar headed goose/Mongolia/143/2005 |
| H12N5 | A/green-winged teal/ALB/199/1991 |
| H13N6 | A/black-headed gull/Sweden/1/1999 |
| H13N8 | A/black-headed gull/Netherlands/1/00 |
| H14N5 | A/Mallard/Astrakhan(Gurjev)/263/1982 |
| H15N2 | A/Australian shelduck/Western Australia/1756/1983 |
| H15N8 | A/duck/AUS/341/1983 |
| H16N3 | A/black-headed gull/Sweden/5/99 |
| H17N10 | A/little yellow-shouldered bat/Guatemala/164/2009 |
| H18N11 | A/flat-faced bat/Peru/033/2010 |
| Influenza B | B/Brisbane/3/2007 |
| | B/Brisbane/60/2008 |
| | B/Florida/07/2004 |
| | B/Florida/4/2006 |
| | B/Hong Kong/05/1972 |
| | B/Malaysia/2506/2004 |
| | B/Massachusetts/03/2010 |
| | B/Ohio/01/2005 |
| | B/PHUKET/3073/2013 |
| | B/Utah/02/2012 |
| | B/Victoria/02/1987 |
| | B/Victoria/504/2000 |
| | B/Wisconsin/01/2012 |
| | B/Yamagata/16/1988 |

In some embodiments, the VLP (e.g. seVLP or smVLP) comprises antigens of 2 or more of H1N1, H1N2, H3N1, H3N2, H2N3, H2N2, H5N1, or H7N9. In some embodiments, the VLP comprises antigens of 3 or more of H1N1, H1N2, H3N1, H3N2, H2N3, H2N2, H5N1, or H7N9. In some embodiments, the VLP comprises antigens of 4 or more of H1N1, H1N2, H3N1, H3N2, H2N3, H2N2, H5N1, or H7N9. In some cases, the VLP is part of a flu vaccine and comprises antigens of 5 or more of H1N1, H1N2, H3N1, H3N2, H2N3, H2N2, H5N1, or H7N9. In some embodiments, the VLP comprises antigens of 6 or more of H1N1, H1N2, H3N1, H3N2, H2N3, H2N2, H5N1, or H7N9. In some embodiments, the VLP comprises antigens of 7 or more of H1N1, H1N2, H3N1, H3N2, H2N3, H2N2, H5N1, or H7N9. In some embodiments, the VLP comprises antigens of H1N1, H1N2, H3N1, H3N2, H2N3, H2N2, H5N1, and H7N9.

In some embodiments, the antigen comprises a Neuraminidase (NA) protein, or a variant or fragment thereof. NA is an influenza virus membrane glycoprotein, and is in some embodiments involved in the destruction of the cellular receptor for the viral HA by cleaving terminal sialic acid residues from carbohydrate moieties on the surfaces of infected cells. NA also in some embodiments cleaves sialic acid residues from viral proteins, preventing aggregation of viruses. NA (along with HA) is one of the two major influenza virus antigenic determinants. The nucleotide and amino acid sequences of some influenza NA proteins are known in the art and are publically available, such as those deposited with the GenBank database.

In some embodiments, the NA comprises a homotetramer. In some embodiments, the NA comprises a subtype have been identified in influenza viruses from birds (N1, N2, N3, N4, N5, N6, N7, N8 or N9). In some embodiments, the NA comprises a Yamagata-like and Victoria-like antigenic lineage. In some embodiments, the NA is involved in the destruction of the cellular receptor for the viral HA by cleaving terminal neuraminic acid (also called sialic acid) residues from carbohydrate moieties on the surfaces of infected cells. In some embodiments, the NA also cleaves sialic acid residues from viral proteins, preventing aggregation of viruses. In some embodiments, the NA facilitates release of viral progeny by preventing newly formed viral particles from accumulating along the cell membrane, as well as by promoting transportation of the virus through the mucus present on the mucosal surface.

Non-limiting, exemplary NA sequences (such as IVA NA found in birds) that are available from GenBank include N1 FJ966084.1, ACP41107.1, HM006761.1, ADD97097.1, AF474048.1, AA033498.1, AY254145.1, AAP21476.1, AY254139.1, AAP21470.1, CY187031.1, AHZ43937.1, CY020887.1, AB052063.1, AY207531.1, AA062045.1, AY207533.1, AA062047.1, AY207528.1, AA062042.1, N5, M24740.1, AAA43672.1, P03478.2, NMIVAA, N6, AY207557.1, AA062071.1, AY207556.1, AA062070.1, AY207553.1, AA062067.1, N7, M38330.1, AAA43425.1, P18881.1, N8, L06575.1, AAA43404.1, AY531038.1, AAT08005.1, CY020903.1, AB052085.1, N9, M17812.1, AAA43575.1, M17813.1, AAA43574.1, AB472040.1, BAH69263.1, NA, AB036870.1, BAB32609.1, NC 002209.1, NP 056663.1, D14855.1, BAA03583.1, AJ419110.1, ACT85965.1, AJ784104.1, AGA18957.1, AJ419111.1, and AA038872.1. Some examples of NA amino acid sequences are provided herein as SEQ ID NOs: 1-4.

In some embodiments, the antigen comprises hemagglutinin (HA), or a variant or fragment thereof. HA is an influenza virus surface glycoprotein. HA mediates binding of the virus particle to a host cells and subsequent entry of the virus into the host cell. In some embodiments, HA also causes red blood cells to agglutinate. The nucleotide and amino acid sequences of numerous influenza HA proteins are known in the art and are publically available, such as those deposited with the GenBank database. HA (along with NA) is one of the two major influenza virus antigenic determinants. Exemplary HA sequences for, for example, 16 HA subtypes from influenza A and examples of HA from influenza B available from the GenBank database. Some examples of HA amino acid sequences are provided herein as SEQ ID NOs: 5-8.

In some embodiments, the antigen comprises HA and a signal sequence. In some embodiments, the HA peptide in the VLP does not include the signal sequence (that is, for example, about amino acids 1-15, 1-16, 1-17, 1-18, or 1-19 of the pre-processed HA protein sequence). In some embodiments, the HA or variant HA (for example when part of a VLP) retains an ability to induce an immune response when administered to a subject, such as a mammal or bird.

In some embodiments, the nucleic acid molecule encoding HA or any other antigen described herein is codon-optimized for expression in mammalian or insect cells. In some embodiments, the nucleic acid molecule is optimized for RNA stability.

In some embodiments, the antigen comprises a matrix protein or an influenza virus matrix protein antigen, or a variant or fragment thereof. Influenza A virus has two matrix proteins, M1 and M2. M1 is a structural protein found within the viral envelope. M1 is a bifunctional membrane/RNA-binding protein that mediates the encapsidation of RNA-nucleoprotein cores into the membrane envelope. M1 consists of two domains connected by a linker sequence. The M2 protein is a single-spanning transmembrane protein that forms tetramers having H+ion channel activity, and when activated by the low pH in endosomes, acidify the inside of the virion, facilitating its uncoating. Homologous proteins in influenza B virus, M1 and BM2, have been described.

The VLP disclosed herein, in addition to comprising, having or presenting an HA subtype or an NA subtype, in some embodiments include an influenza matrix protein, such as M1, M2, or both. In some embodiments, the antigen comprises a matrix protein. In some embodiments, the influenza matrix protein is from the same influenza type as the HA or HA (e.g., if the HA or NA in the VLP is from influenza A, then the matrix protein is from influenza A, but if the HA or NA in the VLP is from influenza B, then the matrix protein is from influenza B). In some embodiments, the matrix peptide sequence present in a VLP provided herein is an influenza A Ml, M2, or M1 and M2 sequence, such as an avian M1, M2, or M1 and M2 sequence, or an influenza B matrix peptide (such as M1, BM2, or both M1 and BM2). In some embodiments, the VLP comprises an influenza A M1 protein (for example if the VLP comprises an influenza A NA or HA protein). In some embodiments, the VLP comprises both an influenza A M1 and an influenza A M2 protein (for example if the VLP comprises an influenza A NA or HA protein). In some embodiments, the VLP comprises an influenza B matrix peptide (for example if the VLP comprises an influenza B NA or HA protein). In some embodiments, the VLP comprises both an influenza B M1 and an influenza B BM2 protein (for example if the VLP comprises an influenza B NA or HA protein).

The nucleotide and amino acid sequences of numerous influenza A M1 and M2 proteins, as well as influenza B matrix proteins, are known in the art and are publically available, such as those deposited with GenBank. Exemplary sequences available from GenBank Some exemplary sequences such as IBV matrix, M1, and M2 sequences include CY002697.1, ABA12718.1, AB189064.1, ABA12719.1, DQ870897.1, AF231361.1, ABS52607.1, AY044171.1, AAD49068.1, ABQ12378.1, AY504605.1, ABS52606.1, ABV53560.1, AB120274.1, AAD49091.1, AAK95902.1, AF100382.1, DQ508916.1, AAT69429.1, BAD29821.1, ABF21318.1, and AHW46771.1. Some examples of matrix or M2 amino acid sequences are provided herein as SEQ ID NOs: 9-12. In some embodiments, the matrix sequences are small M2 membrane proteins, rather than larger, cytoplasmic matrix proteins. In some cases, the larger cytoplasmic matrix proteins are co-expressed to drive budding of particles for traditional VLPs, or the small M2 membrane proteins such as those provided in SEQ ID NOs: 9-12 are used for the VLPs provided herein.

In some embodiments, the antigen or influenza antigen comprises an influenza NB peptide or fragment thereof. Some examples of NB peptide sequences are provided herein as SEQ ID NOs: 13-14. In some embodiments, an influenza virus such as influenza B incorporates two small ion channel transmembrane proteins (NB and BM2) into the virion rather than the one (M2) in influenza A. In some embodiments, the antigen or influenza antigen comprises NB or BM2. In some embodiments, NB is encoded by a nucleic acid such as RNA, and is on the same nucleic acid segment as NA, but in a different reading frame.

Variants of the disclosed influenza HA, NA, M1 and M2 proteins and coding sequences disclosed herein are in some embodiments characterized by possession of at least about 80%, at least 90%, at least 95%, at least 96%, at least 97%, at least 98% or at least 99% sequence identity counted over the full-length alignment with the amino acid sequence using the NCBI Blast 2.0, gapped blastp set to default parameters. For comparisons of amino acid sequences of greater than about 30 amino acids, the Blast 2 sequences function employed in some embodiments using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with greater similarity to the reference sequences will show increasing percentage identities when assessed by this method, such as at least 95%, at least 98%, or at least 99% sequence identity. In some embodiments, when less than the entire sequence is compared for sequence identity, homologs and variants will in some embodiments possess at least 80% sequence identity over short windows of 10-20 amino acids, and in some embodiments possess sequence identities of at least 85% or at least 90% or at least 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided. Thus, a variant influenza HA, NA, or matrix protein (or coding sequence) has in some embodiments at least 80%, at least 85%, at least 90%, at least 91%, at least 92%, at least 95%, at least 96%, at least 97%, at least 98% or at least 99% sequence identity to any antigen or antigen sequence provided herein and are in some embodiments used in the methods and compositions provided herein.

In some embodiments, the VLP presents or comprises an influenza A HA or influenza ANA protein, in combination with influenza A M1, influenza A M2, or both influenza A M1 and influenza A M2 proteins. In other embodiments herein, an influenza VLP presents or comprises an influenza B HA or influenza B NA protein, in combination with influenza B matrix protein M1 or both influenza B M1 and BM2 proteins.

2. Coronavirus Antigens

Disclosed herein, in some embodiments, are VLPs comprising an antigen. In some embodiments, the antigen is a coronavirus antigen, or a variant or fragment thereof. In some embodiments, the fragment is a functional fragment. In some embodiments, the antigen is a coronavirus antigen. In some embodiments, the antigen is a variant or a coronavirus antigen. In some embodiments, the antigen is a fragment or a coronavirus antigen.

The coronavirus antigen may be from a coronavirus. Non-limiting examples of coronaviruses include MHV, HCoV-0C43, AIBV, BcoV, TGV, FIPV, HCoV-229E, MERS virus, severe acute respiratory syndrome coronavirus 1 (SARS-CoV-1), or SARS-CoV-2. In some embodiments, the coronavirus is a MERS virus. In some embodiments, the coronavirus is a SARS coronavirus. In some embodiments, the coronavirus is a SARS-CoV-1. In some embodiments, the coronavirus is a SARS-CoV-2. In some embodiments, the coronavirus comprises SARS-CoV-2.

In some embodiments, the coronavirus causes a viral infection. For example, the SARS coronavirus may cause a SARS infection. In some embodiments, SARS-CoV-2 causes coronavirus disease 2019. In some embodiments, the viral infection is a coronavirus infection. In some embodiments, the viral infection is coronavirus disease 2019 (COVID-19). In some embodiments, the subject has the viral infection. In some embodiments, the subject has COVID-19.

In some embodiments, the coronavirus antigen is a coronavirus protein. In some embodiments, the antigen comprises a coronavirus protein, or a fragment thereof. In some embodiments, the antigen comprises a coronavirus protein. In some embodiments, the coronavirus protein comprises a spike (S) protein, an envelope (E) protein, a membrane protein (M), or a nucleocapsid (N) protein. In some embodiments, the coronavirus protein comprises a spike (S) protein. In some embodiments, the coronavirus protein comprises a envelope (E) protein. In some embodiments, the coronavirus protein comprises a membrane protein (M). In some embodiments, the coronavirus protein comprises a nucleocapsid (N) protein. In some embodiments, the coronavirus protein comprises S1 or S2. In some embodiments, the spike protein is cleaved into S1 and/or S2. In some embodiments, the spike protein includes S 1. In some embodiments, the spike protein includes S2. In some embodiments, the coronavirus protein is recombinant and/or non-naturally occurring. In some embodiments, the spike protein is a functional spike protein, or a functional fragment thereof. In some embodiments, the spike protein binds to a receptor. In some embodiments, the spike protein fragment binds to a receptor. In some embodiments, the receptor comprises an ACE2. In some embodiments, the receptor is angiotensin ACE2. In some embodiments, the spike protein binds to ACE2. In some embodiments, the spike protein fragment binds to ACE2. In some embodiments, the receptor is a human protein. In some embodiments, the receptor is a human ACE2. In some embodiments, upon binding to the human receptor the spike protein is capable of being internalized into a cell.

In some embodiments, the coronavirus protein comprises an amino acid sequence that is 75.0%, 80.0%, 85.0%, 90.0%, 91.0%, 92.0%, 93.0%, 94.0%, 95.0%, 96.0%, 97.0%, 97.5%, 98.0%, 98.5%, 99.0%, 99.5%, 99.9%, 100%, or a range of percentages defined by any two of the aforementioned percentages, identical to any of SEQ ID NOs: 20-29, or a fragment thereof. In some embodiments, the coronavirus protein comprises an amino acid sequence that is 75.0%, 80.0%, 85.0%, 90.0%, 91.0%, 92.0%, 93.0%, 94.0%, 95.0%, 96.0%, 97.0%, 97.5%, 98.0%, 98.5%, 99.0%, 99.5%, 99.9%, 100%, or a range of percentages defined by any two of the aforementioned percentages, identical to any of SEQ ID NOs: 20-29.

In some embodiments, the coronavirus protein comprises an amino acid sequence that is at least 75.0% identical, at least 80.0% identical, at least 85.0% identical, at least 90.0% identical, at least 91.0% identical, at least 92.0% identical, at least 93.0% identical, at least 94.0% identical, at least 95.0% identical, at least 96.0% identical, at least 97.0% identical, at least 97.5% identical, at least 98.0% identical, at least 98.5% identical, at least 99.0% identical, at least 99.5% identical, at least 99.9% identical, or 100% identical, to any of SEQ ID NOs: 20-29.

In some embodiments, the coronavirus protein comprises an amino acid sequence that is no more than 75.0% identical, no more than 80.0% identical, no more than 85.0% identical, no more than 90.0% identical, no more than 91.0% identical, no more than 92.0% identical, no more than 93.0% identical, no more than 94.0% identical, no more than 95.0% identical, no more than 96.0% identical, no more than 97.0% identical, no more than 97.5% identical, no more than 98.0% identical, no more than 98.5% identical, no more than 99.0% identical, no more than 99.5% identical, no more than 99.9% identical, or 100% identical, to any of SEQ ID NOs: 20-29.

In some embodiments, the coronavirus protein comprises an amino acid sequence that is 75.0% identical, 80.0% identical, 85.0% identical, 90.0% identical, 91.0% identical, 92.0% identical, 93.0% identical, 94.0% identical, 95.0% identical, 96.0% identical, 97.0% identical, 97.5% identical, 98.0% identical, 98.5% identical, 99.0% identical, 99.5% identical, 99.9% identical, or 100% identical to SEQ ID NO: 20 or a fragment thereof, or comprises an amino acid sequence comprising a range of percent identities compared to SEQ ID NO: 20 or a fragment thereof. In some embodiments, the coronavirus protein comprises the sequence of SEQ ID NO: 20. In some embodiments, the coronavirus protein comprises the sequence of a fragment of SEQ ID NO: 20.

In some embodiments, the coronavirus protein comprises an amino acid sequence that is 75.0% identical, 80.0% identical, 85.0% identical, 90.0% identical, 91.0% identical, 92.0% identical, 93.0% identical, 94.0% identical, 95.0% identical, 96.0% identical, 97.0% identical, 97.5% identical, 98.0% identical, 98.5% identical, 99.0% identical, 99.5% identical, 99.9% identical, or 100% identical to SEQ ID NO: 21 or a fragment thereof, or comprises an amino acid sequence comprising a range of percent identities compared to SEQ ID NO: 21 or a fragment thereof. In some embodiments, the coronavirus protein comprises the sequence of SEQ ID NO: 21. In some embodiments, the coronavirus protein comprises the sequence of a fragment of SEQ ID NO: 21.

In some embodiments, the coronavirus protein comprises an amino acid sequence that is 75.0% identical, 80.0% identical, 85.0% identical, 90.0% identical, 91.0% identical, 92.0% identical, 93.0% identical, 94.0% identical, 95.0% identical, 96.0% identical, 97.0% identical, 97.5% identical, 98.0% identical, 98.5% identical, 99.0% identical, 99.5% identical, 99.9% identical, or 100% identical to SEQ ID NO: 22 or a fragment thereof, or comprises an amino acid sequence comprising a range of percent identities compared to SEQ ID NO: 22 or a fragment thereof. In some embodiments, the coronavirus protein comprises the sequence of SEQ ID NO: 22. In some embodiments, the coronavirus protein comprises the sequence of a fragment of SEQ ID NO: 22.

In some embodiments, the coronavirus protein comprises an amino acid sequence that is 75.0% identical, 80.0% identical, 85.0% identical, 90.0% identical, 91.0% identical, 92.0% identical, 93.0% identical, 94.0% identical, 95.0% identical, 96.0% identical, 97.0% identical, 97.5% identical, 98.0% identical, 98.5% identical, 99.0% identical, 99.5% identical, 99.9% identical, or 100% identical to SEQ ID NO: 23 or a fragment thereof, or comprises an amino acid sequence comprising a range of percent identities compared to SEQ ID NO: 23 or a fragment thereof. In some embodiments, the coronavirus protein comprises the sequence of SEQ ID NO: 23. In some embodiments, the coronavirus protein comprises the sequence of a fragment of SEQ ID NO: 23.

In some embodiments, the coronavirus protein comprises an amino acid sequence that is 75.0% identical, 80.0% identical, 85.0% identical, 90.0% identical, 91.0% identical, 92.0% identical, 93.0% identical, 94.0% identical, 95.0% identical, 96.0% identical, 97.0% identical, 97.5% identical, 98.0% identical, 98.5% identical, 99.0% identical, 99.5% identical, 99.9% identical, or 100% identical to SEQ ID NO: 24 or a fragment thereof, or comprises an amino acid sequence comprising a range of percent identities compared to SEQ ID NO: 24 or a fragment thereof. In some embodiments, the coronavirus protein comprises the sequence of SEQ ID NO: 24. In some embodiments, the coronavirus protein comprises the sequence of a fragment of SEQ ID NO: 24.

In some embodiments, the coronavirus protein comprises an amino acid sequence that is 75.0% identical, 80.0% identical, 85.0% identical, 90.0% identical, 91.0% identical, 92.0% identical, 93.0% identical, 94.0% identical, 95.0% identical, 96.0% identical, 97.0% identical, 97.5% identical, 98.0% identical, 98.5% identical, 99.0% identical, 99.5% identical, 99.9% identical, or 100% identical to SEQ ID NO: 25 or a fragment thereof, or comprises an amino acid sequence comprising a range of percent identities compared to SEQ ID NO: 25 or a fragment thereof. In some embodiments, the coronavirus protein comprises the sequence of SEQ ID NO: 25. In some embodiments, the coronavirus protein comprises the sequence of a fragment of SEQ ID NO: 25.

In some embodiments, the coronavirus protein comprises an amino acid sequence that is 75.0% identical, 80.0% identical, 85.0% identical, 90.0% identical, 91.0% identical, 92.0% identical, 93.0% identical, 94.0% identical, 95.0% identical, 96.0% identical, 97.0% identical, 97.5% identical, 98.0% identical, 98.5% identical, 99.0% identical, 99.5% identical, 99.9% identical, or 100% identical to SEQ ID NO: 26 or a fragment thereof, or comprises an amino acid sequence comprising a range of percent identities compared to SEQ ID NO: 26 or a fragment thereof. In some embodiments, the coronavirus protein comprises the sequence of SEQ ID NO: 26. In some embodiments, the coronavirus protein comprises the sequence of a fragment of SEQ ID NO: 26.

In some embodiments, the coronavirus protein comprises an amino acid sequence that is 75.0% identical, 80.0% identical, 85.0% identical, 90.0% identical, 91.0% identical, 92.0% identical, 93.0% identical, 94.0% identical, 95.0% identical, 96.0% identical, 97.0% identical, 97.5% identical, 98.0% identical, 98.5% identical, 99.0% identical, 99.5% identical, 99.9% identical, or 100% identical to SEQ ID NO: 27 or a fragment thereof, or comprises an amino acid sequence comprising a range of percent identities compared to SEQ ID NO: 27 or a fragment thereof. In some embodiments, the coronavirus protein comprises the sequence of SEQ ID NO: 27. In some embodiments, the coronavirus protein comprises the sequence of a fragment of SEQ ID NO: 27.

In some embodiments, the coronavirus protein comprises an amino acid sequence that is 75.0% identical, 80.0% identical, 85.0% identical, 90.0% identical, 91.0% identical, 92.0% identical, 93.0% identical, 94.0% identical, 95.0% identical, 96.0% identical, 97.0% identical, 97.5% identical, 98.0% identical, 98.5% identical, 99.0% identical, 99.5% identical, 99.9% identical, or 100% identical to SEQ ID NO: 28 or a fragment thereof, or comprises an amino acid sequence comprising a range of percent identities compared to SEQ ID NO: 28 or a fragment thereof. In some embodiments, the coronavirus protein comprises the sequence of SEQ ID NO: 28. In some embodiments, the coronavirus protein comprises the sequence of a fragment of SEQ ID NO: 28.

In some embodiments, the coronavirus protein comprises an amino acid sequence that is 75.0% identical, 80.0% identical, 85.0% identical, 90.0% identical, 91.0% identical, 92.0% identical, 93.0% identical, 94.0% identical, 95.0% identical, 96.0% identical, 97.0% identical, 97.5% identical, 98.0% identical, 98.5% identical, 99.0% identical, 99.5% identical, 99.9% identical, or 100% identical to SEQ ID NO: 29 or a fragment thereof, or comprises an amino acid sequence comprising a range of percent identities compared to SEQ ID NO: 29 or a fragment thereof. In some embodiments, the coronavirus protein comprises the sequence of SEQ ID NO: 29. In some embodiments, the coronavirus protein comprises the sequence of a fragment of SEQ ID NO: 29.

In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to any of SEQ ID NOs: 20-29, or a fragment thereof. In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to any of SEQ ID NOs: 20-29.

In some embodiments, the coronavirus protein comprises an amino acid sequence that has no more than 1, 2, 3, 4, 5, 6, 7, 8, 9, 10, 20, 40, or a range defined by any of the aforementioned integers, amino acid substitutions, deletions, and/or insertions, compared to any of SEQ ID NOs: 20-29, or a fragment thereof. In some embodiments, the coronavirus protein comprises an amino acid sequence that has no more than 1, 2, 3, 4, 5, 6, 7, 8, 9, 10, 20, 40, or a range defined by any of the aforementioned integers, amino acid substitutions, deletions, and/or insertions, compared to any of SEQ ID NOs: 20-29.

In some embodiments, the coronavirus protein comprises an amino acid sequence that has at least 1, 2, 3, 4, 5, 6, 7, 8, 9, 10, 20, 40, or a range defined by any of the aforementioned integers, amino acid substitutions, deletions, and/or insertions, compared to any of SEQ ID NOs: 20-29, or a fragment thereof. In some embodiments, the coronavirus protein comprises an amino acid sequence that has at least 1, 2, 3, 4, 5, 6, 7, 8, 9, 10, 20, 40, or a range defined by any of the aforementioned integers, amino acid substitutions, deletions, and/or insertions, compared to any of SEQ ID NOs: 20-29.

In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 20, or a fragment thereof. In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 20.

In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 21, or a fragment thereof. In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 21.

In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 22, or a fragment thereof. In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 22.

In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 23, or a fragment thereof. In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 23.

In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 24, or a fragment thereof. In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 24.

In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 25, or a fragment thereof. In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 25.

In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 26, or a fragment thereof. In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 26.

In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 27, or a fragment thereof. In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 27.

In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 28, or a fragment thereof. In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 28.

In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 29, or a fragment thereof. In some embodiments, the coronavirus protein comprises an amino acid sequence that has 1, 2, 3, 4, 5, 6, 7, 8, 9, 10, 20, 40, or a range defined by any of the aforementioned integers, amino acid substitutions, deletions, and/or insertions, compared to SEQ ID NO: 29.

3. Polyvalent VLPs

Provided herein are vaccines that contain two or more different VLPs (e.g. seVLPs or smVLPs), such as two or more different VLP populations. Such vaccines are referred to as polyvalent VLPs (or polyvalent VLP-containing vaccines). In some embodiments, the vaccines comprise VLPs comprising different antigens. In some embodiments, the vaccines comprise VLPs comprising different influenza hemagglutinin (HA) polypeptides, such as a first VLP that contains or comprises a first HA polypeptide, and a second VLP that contains or comprises a second HA polypeptide, wherein the first and second HA polypeptides are different subtypes (or are from different influenza viruses, such as influenza A and B). In some embodiments, the vaccine contains a plurality of different VLPs, each comprising or containing a different HA subtype or HA from a different influenza (e.g., A and B). In some embodiments, the VLPs include other reagents, such as a pharmaceutically acceptable carrier and/or an adjuvant.

In some embodiments, the disclosed vaccines include a polyvalent mixture of influenza VLPs each containing a single HA subtype from influenza A or B. In some embodiments, the vaccines further include VLPs containing influenza A or B NA proteins (e.g., additional VLP populations each comprising an influenza A NA subtype or influenza B NA). In some embodiments, the VLPs also contain influenza A or B matrix proteins. In some embodiments, VLPs comprising influenza A NA or HA comprise influenza A M1, M2 or both, while VLPs comprising influenza B NA or HA comprise an influenza B matrix protein, such as influenza B M1, BM2, or both. Intranasal, intradermal, systemic, or intravenous delivery or administration is used in some embodiments to induce mucosal and systemic immunity. In some embodiments, the monovalent or polyvalent VLPs are non-infectious, safe, and easy to manufacture and use. In some embodiments, the polyvalent VLPs (which in some embodiments include mixtures of VLP populations comprising influenza A or B HA), are used to provide a broadly reactive seasonal vaccine.

In some embodiments, the vaccine comprises at least two different VLPs, such as at least two different populations of VLPs, each VLP or VLP population containing one HA subtype (or containing an HA from one influenza virus, such as influenza A and B). Some embodiments include a first VLP that contains a first HA subtype (H—X) and a second VLP that contains a different HA subtype (H—Y). In some embodiments, the first VLP contains a first HA from influenza B (H—X) and the second VLP contain a second but different HA from influenza B (H—Y), or the first VLP contains a first HA from influenza A (H—X) and the second VLP contains a second but different HA from influenza A (H—Y). In some embodiments, the first VLP contains a first HA from influenza A (H—X) and the second VLP contains a second HA from influenza B (H—Y). In some embodiments, each VLP contains a plurality of VLPs, each population containing a different HA subtype (or HA from a different influenza virus).

In some embodiments, more than two different VLPs or vaccines are included in the vaccine. In some embodiments, the vaccine comprises at least 2, at least 3, at least 4, at least 5, at least 6, at least 7, at least 8, at least 9, or at least 10, such as 2, 3, 4, 5, 6, 7, 8, 9, 10, 11, 12, 13, 14, 15, 16, 17, or 18 different VLPs or VLP populations, each comprising a different antigen. In some embodiments, the different antigens are each from a different influenza HA subtype and/or from a different influenza virus, such as 2-8, 2-6, 5-6, or 4-6 different VLPs or VLP populations (wherein each VLP or VLP population has a different HA protein subtype and/or HA from a different virus). In some embodiments, a first VLP comprises a first influenza A HA polypeptide selected from the group consisting of HA subtype H1, H2, H3, H4, H5, H6, H7, H8, H9, H10, H11, H12, H13, H14, H15 and H16; while a second VLP comprises a second influenza A HA polypeptide selected from the group consisting of HA subtype H1, H2, H3, H4, H5, H6, H7, H8, H9, H10, H11, H12, H13, H14, H15 and H16, wherein the first and the second HA polypeptide are different subtypes. Thus, if the vaccine included a third VLP, such as a third VLP population, the third influenza A HA polypeptide would be selected from the group consisting of HA subtype H1, H2, H3, H4, H5, H6, H7, H8, H9, H10, H11, H12, H13, H14, H15 and H16, wherein the third HA polypeptide subtype is different from the first and the second HA polypeptide subtypes.

In some embodiments, a first VLP comprises a first influenza A HA polypeptide selected from the group consisting of HA subtype H1, H2, H3, H4, H5, H6, H7, H8, H9, H10, H11, H12, H13, H14, H15 and H16; while a second VLP comprises a first influenza B HA polypeptide such as Yamagata-like or Victoria-like antigens. If the vaccine included a third VLP, such as a third VLP population containing a second influenza A HA polypeptide, it would be selected from the group consisting of HA subtype H1, H2, H3, H4, H5, H6, H7, H8, H9, H10, H11, H12, H13, H14, H15 and H16, wherein the second influenza A HA polypeptide subtype is different from the first influenza A HA polypeptide subtype. If the vaccine included a third VLP, such as a third VLP population containing a second influenza B HA polypeptide, the second influenza B HA would be different from the first influenza B HA. In a specific example, the vaccine comprises at least two, at least three, at least four, at least five, such as 2, 3, 4, 5, 6, 7, 8, 9 or 10 different VLPs (or VLP populations), wherein at least one VLP population comprises an influenza A HA subtype, at least one VLP population comprises an influenza B HA, and optionally at least one VLP population comprises an influenza A NA subtype.

In some embodiments, the vaccine comprises separate VLPs (or VLP populations). In some embodiments, a first VLP population comprises influenza A H1, a second VLP population comprises influenza A H3, a third VLP population comprises influenza A H5, a fourth VLP population comprises influenza A H7, a fifth VLP population comprises influenza A N1, a sixth VLP population comprises influenza A N2, a seventh VLP population comprises influenza B Yamagata-like or Victoria-like antigen, and optionally an eighth VLP population comprises influenza B Yamagata-like or Victoria-like antigen (that is different from the seventh VLP population. In some embodiments, a vaccine is used as a seasonal vaccine or as a prepandemic vaccine.

In some embodiments, there are two major groups of influenza A virus HAs: group 1 contains H1, H2, H5, H6, H8, H9, H11, H12, H13, and H16, and group 2 contains H3, H4, H7, H10, H14, and H15 subtypes. In some embodiments, the vaccine comprises a first VLP or first population of VLPs comprising at least one HA polypeptide of Group 1 (e.g., H1, H2, H5, H6, H8, H9, H11, H12, H13, or H16), and a second VLP or second population of VLPs comprising at least one HA polypeptide of Group 2 (e.g., H3, H4, H7, H10, H14, or H15). In another example, the vaccine comprises at least two different VLPs or different populations of VLPs, each comprising a different HA polypeptide of Group 1 (e.g., H1, H2, H5, H6, H8, H9, H11, H12, H13, or H16). In another example, the vaccine comprises at least two different VLPs or different populations of VLPs, each comprising a different HA polypeptide of Group 2 (e.g., H3, H4, H7, H10, H14, or H15). Similarly, while influenza B virus HA does not have distinct subtypes, there are two major antigenic lineages, Victoria-like and Yamagata-like that are also phylogenetically distinct.

In some embodiments, the vaccine comprises at least two, at least three, at least four, at least five, such as 2, 3, 4, 5, 6, 7, 8, 9 or 10 different VLPs (or VLP populations), each containing a different influenza A HA polypeptide of Group 1 (e.g., H1, H2, H5, H6, H8, H9, H11, H12, H13, or H16). In a specific example, the vaccine comprises at least two, at least three, at least four, at least five, at least six, such as 2, 3, 4, 5, or 6, different VLPs (or VLP populations), each containing a different influenza A HA polypeptide of Group 2 (e.g., H3, H4, H7, H10, H14, or H15).

In some embodiments, the first influenza A HA polypeptide is HA subtype H1, H2 or H5 and the second influenza A HA polypeptide is HA subtype H3, H7 or H9. In another specific example, the first influenza A HA polypeptide is HA subtype H1, H2, H3, H5, H7 or H9 and the second influenza A HA polypeptide is HA subtype H1, H2, H3, H5, H7 or H9, wherein the first and the second HA polypeptide are different subtypes. In some embodiments, (i) the first influenza A HA polypeptide is HA subtype H2 and the second influenza A HA polypeptide is HA subtype H5; (ii) the first influenza A HA polypeptide is HA subtype H3 and the second influenza A HA polypeptide is HA subtype H7; (iii) the first influenza A HA polypeptide is HA subtype H1 and the second influenza A HA polypeptide is HA subtype H3; (iv) the first influenza A HA polypeptide is HA subtype H2 and the second influenza A HA polypeptide is HA subtype H7; (v) the first influenza A HA polypeptide is HA subtype H5 and the second influenza A HA polypeptide is HA subtype H3; or (vi) the first influenza A HA polypeptide is HA subtype H1 and the second influenza A HA polypeptide is HA subtype H7.

In some embodiments, the vaccine comprises at least four different populations of VLPs, wherein the first population of VLPs comprises influenza A HA subtype H1, the second population of VLPs comprises influenza A HA subtype H3, the third population of VLPs comprises influenza A HA subtype H5, and the fourth population of VLPs comprises influenza A HA subtype H7. In some embodiments, the vaccine further comprises a fifth population of VLPs comprising influenza A HA subtype H9. In some embodiments, the vaccine further comprises a sixth population of VLPs comprising an influenza ANA, such as N1 or N2. In some embodiments, the vaccine further comprises a seventh and eighth population of VLPs comprising influenza A NA N1 (seventh population) and N2 (eighth population). Such VLPs In some embodiments, also include M1 and M2.

In some embodiments, the VLPs of the disclosure in addition to having an HA protein, comprise an influenza matrix protein (e.g., influenza A M1, influenza A M2, or both). In some embodiments, the vaccine 106 comprises a VLP or VLP population having a first HA subtype H—X and matrix protein M1 and VLP or VLP population having a second HA subtype H—Y and matrix protein M1. In some embodiments, M2 is present in VLP and/or VLP population. In some embodiments, the VLP or VLP population contains a first HA from influenza A (H—X) and an influenza A matrix protein such as M1 or M2, and the second VLP or VLP population contains a second HA from influenza B (H—Y) and an influenza B matrix protein.

Some embodiments, in addition to comprising VLPs comprising HA, include a VLP (or population of VLPs) that comprises an influenza neuraminidase (NA) polypeptide. In some embodiments, the vaccine comprises two or more different VLPs or VLP populations, each having a different influenza NA polypeptide. In some embodiments, the vaccine comprises a first VLP comprising a first influenza NA polypeptide, a second VLP comprising a second influenza NA polypeptide, or both, wherein the first and the second NA polypeptide are different subtypes or are from different influenza viruses. In some embodiments, the vaccine comprises VLP or VLP populations, each having a different HA subtype (or NA from a different influenza virus), and further comprises VLP or VLP population having NA subtype N-X. In some embodiments, the VLPs or vaccine comprises an influenza matrix protein (e.g., M1, M2, or both).

Phylogenetically, there are two groups of influenza A virus NAs that form two groups: group 1 contains N1, N4, N5, and N8, and group 2 contains N2, N3, N6, N7, and N9. Thus, in one example, the polyvalent VLP-containing vaccine further comprises a first VLP or first population of VLPs containing at least one NA polypeptide of Group 1 (e.g., N1, N4, N5, or N8), and a second VLP or second population of VLPs containing at least one NA polypeptide of Group 2 (e.g., N2, N3, N6, N7, or N9). In another example, the polyvalent VLP-containing vaccine further comprises at least two different VLPs or different populations of VLPs, each containing a different NA polypeptide of Group 1 (e.g., N1, N4, N5, or N8). In another example, the polyvalent VLP-containing vaccine further comprises at least two different VLPs or different populations of VLPs, each containing a different NA polypeptide of Group 2 (e.g., N2, N3, N6, N7, or N9).

In some embodiments, the polyvalent VLP-containing vaccine further comprises 1, 2, 3, or 4 different VLPs (or VLP populations), each containing a different NA polypeptide of Group 1 (e.g., N1, N4, N5, and N8). In a specific example, the vaccine comprises 1, 2, 3, 4, or 5, different VLPs (or VLP populations), each containing a different NA polypeptide of Group 2 (e.g., N2, N3, N6, N7, or N9).

Similarly, while influenza B virus NA does not have distinct subtypes, there are two major antigenic lineages, Victoria-like and Yamagata-like that are also phylogenetically distinct. In some embodiments, the polyvalent VLP-containing vaccine further comprises a first VLP or first population of VLPs containing at least one influenza B NA polypeptide (e.g., Victoria-like), and a second VLP or second population of VLPs containing at least one influenza B NA polypeptide (e.g., Yamagata-like).

In some embodiments, the NA-VLPs of the disclosure in addition to having an NA protein, include an influenza matrix protein (e.g., influenza A M1, influenza A M2, or both; or influenza B M1, influenza B BM2, or both).

In some embodiments, the vaccine comprises a first population of VLPs comprising influenza A HA subtype H1, a second population of VLPs comprising influenza A HA subtype H3, a third population of VLPs comprising influenza A HA subtype H5, and a fourth population of VLPs comprising influenza A HA subtype H7. In some embodiments, the vaccine further or optionally comprises a fifth population of VLPs comprising influenza A HA subtype H9. In some embodiments, the vaccine further comprises a sixth population of VLPs comprising an influenza ANA, such as N1 or N2. In some embodiments, the vaccine further comprises a sixth and seventh population of VLPs comprising influenza A NA N1 (sixth population) and N2 (seventh population). In some embodiments, the vaccine further comprises a eighth VLP population that comprises influenza B Yamagata-like or Victoria-like antigen, and optionally a ninth VLP population comprises influenza B Yamagata-like or Victoria-like antigen (that is different from the eighth VLP population). In some embodiments, such a vaccine is used as a seasonal vaccine or as a prepandemic vaccine.

C. Anchor Molecules

Disclosed herein, in certain embodiments, are seVLPs comprising or consisting of (a) a synthetic lipid vesicle comprising a lipid bilayer comprising an inner surface and an outer surface; (b) an anchor molecule embedded in the lipid bilayer; and (c) an antigen bound to the anchor molecule. Also disclosed herein, in certain embodiments, are smVLPs comprising a synthetic, semisynthetic or natural lipid bilayer comprising a first side and a second side; an anchor molecule embedded in the lipid bilayer; and an antigen bound to the anchor molecule. In some embodiments, the VLPs are stable at room temperature.

In some embodiments, the anchor molecule comprises a transmembrane protein, a lipid-anchored protein, or a fragment or domain thereof.

In some embodiments, the anchor molecule comprises a hydrophobic moiety. In some embodiments, the anchor molecule comprises a prenylated protein, fatty acylated protein, a glycosylphosphatidylinositol-linked protein, or a fragment thereof.

In some embodiments, the anchor molecule comprises a hydrophobic transmembrane domain, a glycosylphosphatidylinositol attachment, or another structural feature that assists in localizing the antigen to the membrane such as a protein-protein association domain, a lipid association domain, a glycolipid association domain, or a proteoglycan association domain, for example, a cell surface receptor binding domain, an extracellular matrix binding domain, or a lipid raft-associating domain.

In some embodiments, the anchor molecule comprises a transmembrane polypeptide domain. In some embodiments, the transmembrane polypeptide domain comprises a membrane spanning domain (such as an [a]-helical domain) which comprises a hydrophobic region capable of energetically favorable interaction with the phospholipid fatty acyl tails that form the interior of the plasma membrane bilayer, or a membrane-inserting domain polypeptide that in some embodiments comprise a membrane-inserting domain which comprises a hydrophobic region capable of energetically favorable interaction with the phospholipid fatty acyl tails that form the interior of the plasma membrane bilayer but that in some embodiments do not span the entire membrane. Some examples of transmembrane proteins having one or more transmembrane polypeptide domains include members of the integrin family, CD44, glycophorin, MHC Class I and Il glycoproteins, EGF receptor, G protein coupled receptor (GPCR) family, receptor tyrosine kinases (such as insulin-like growth factor 1 receptor (IGFR) and platelet-derived growth factor receptor (PDGFR)), porin family and other transmembrane proteins. Some embodiments include use of a portion of a transmembrane polypeptide domain such as a truncated polypeptide having membrane-inserting characteristics.

In some embodiments, the anchor molecule comprises a protein-protein association domain, for example a protein-protein association domain that is capable of specifically associating with an extracellularly disposed region of a cell surface protein or glycoprotein. In some embodiments, the protein-protein association domain results in an association that is initiated intracellularly, for instance, concomitant with the synthesis, processing, folding, assembly, transport and/or export to the cell surface of a cell surface protein. In some embodiments, the protein-protein association domain is known to associate with another cell surface protein that is membrane anchored and exteriorly disposed on a cell surface. Non-limiting examples of such domains include, RGD-containing polypeptides comprising those that are capable of integrin.

In some embodiments, sequences encoding the anchor molecule or transmembrane domain are included in a polynucleotide to provide surface expression of the antigen or a fusion protein that comprises the antigen and anchor molecule. In some embodiments, the fusion protein is cloned in-frame with a selectable marker to allow for the selection of productive in-frame products.

### IV. VACCINES

Disclosed herein, in certain embodiments, are vaccines comprising (a) a VLP (e.g. seVLP or smVLP), and (b) an excipient, carrier or adjuvant.

In some embodiments, the vaccine contains at least one excipient. In some embodiments, the excipient is an antiadherent, a binder, a coating, a color or dye, a disintegrant, a flavor, a glidant, a lubricant, a preservative, a sorbent, a sweetener, or a vehicle. In some embodiments, the excipient comprises a wetting or emulsifying agent, or a pH buffering agent. In some embodiments, the excipient contains pharmaceutically acceptable salts to adjust the osmotic pressure, buffers, preservatives and the like.

In some embodiments, the excipient comprises sodium alginate. In some embodiments, the excipient comprises alginate microspheres. In some embodiments, the excipient comprises carbopol, for example in combination with starch. In some embodiments, the excipient comprises chitosan, a non-toxic linear polysaccharide that is produced by chitin deacetylation. In one example the chitosan is in the form of chitosan nanoparticles, such as N-trimethyl chitosan (TMC)-based nanoparticles.

In some embodiments, excipient comprises wetting or emulsifying agents, or pH buffering agents. In some embodiments, the excipient comprises one or more lipopeptides of bacterial origin, or their synthetic derivatives, such as Pam3Cys, (Pam2Cys, single/multiple-chain palmitic acids and lipoamino acids (LAAs). In some embodiments, the vaccine contains one or more adjuvants, for example a mucosal adjuvant, such as one or more of CpG oligodeoxynucleotides (CpG ODN), Flt3 ligand, and MLA. In some embodiments, the adjuvant comprises a clinical grade MLA formulation, such as MPL (3-O-desacyl-4′-monophosphoryl lipid A) adjuvant. In some embodiments, the vaccine contains a pharmaceutically acceptable carrier and an adjuvant, such as a mucosal adjuvant, for example as one or more of CpG oligodeoxynucleotides, Flt3 ligand, and MLA. In one example, the adjuvant comprises MLA, such as a clinical grade formulation, for example MPL (3-O--desacyl-4′-monophosphoryl lipid A) adjuvant. In some embodiments, the vaccine contains one or more adjuvants, such as lipid A monophosphoryl (MPL), Flt3 ligand, immunostimulatory oligonucleotides (such as CpG oligonucleotides), or combinations thereof In some embodiments, the adjuvant comprises a TLR agonist such as imiquimod, Flt3 ligand, MLA, or an immunostimulatory oligonucleotide such as a CpG oligonucleotide. In some embodiments, the adjuvant is imiquimod.

In some embodiments, the vaccine contains at least one adjuvant. As used here, an “adjuvant” is a substance or vehicle that non-specifically enhances the immune response to an antigen (e.g., influenza HA and/or NA). In some embodiments, the adjuvant is used with the VLPs disclosed herein. In some embodiments, the adjuvant comprises a suspension of minerals (alum, aluminum hydroxide, or phosphate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. In some embodiments, immunostimulatory oligonucleotides (such as those comprising a CpG motif) are used as adjuvants. Some examples of adjuvants include biological molecules, such as costimulatory molecules. Exemplary biological adjuvants include IL-2, RANTES, GM-CSF, TNF-alpha., IFN-gamma., G-CSF, LFA-3, CD72, B7-1, B7-2, OX-40L and 41 BBL. In some embodiments, the adjuvant is one or more a TLR agonists, such as an agonist of TLR1/2 (which is in some embodiments a synthetic ligand) (e.g., Pam3Cys), TLR2 (e.g., CFA, Pam2Cys), TLR3 (e.g., polyI:C, poly A:U), TLR4 (e.g., MPLA, Lipid A, and LPS), TLRS (e.g., flagellin), TLR7 (e.g., gardiquimod, imiquimod, loxoribine, Resiquimod), TLR7/8 (e.g., R848), TLR8 (e.g., imidazoquionolines, ssPolyU, 3M-012), TLR9 (e.g., ODN 1826 (type B), ODN 2216 (type A), CpG oligonucleotides) and/or TLR11/12 (e.g., profilin). In some embodiments, the adjuvant is lipid A, such as lipid A monophosphoryl (MPL) from Salmonella enterica serotype Minnesota Re 595.

In some embodiments, the vaccine contains at least one pharmaceutically acceptable carrier. In some embodiments, the carrier is saline, buffered saline, dextrose, water, glycerol, sesame oil, ethanol, and combinations thereof. In some embodiments, the pharmaceutically acceptable carrier is determined in part by the particular vaccine being administered, and/or by the particular method used to administer the vaccine. Pharmaceutically acceptable carriers include, but are not limited to, saline, buffered saline, dextrose, water, glycerol, sesame oil, ethanol, and combinations thereof. In some embodiments, the carrier is sterile, and the formulation suits the mode of administration. In some embodiments, the vaccine contains a liquid solution, suspension, emulsion, tablet, pill, capsule, sustained release formulation, or powder.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, comprising saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. In some embodiments, preservatives or other additives are present such as, for example, antimicrobials, anti-oxidants, chelating agents, and inert gases and the like.

In some embodiments, the carrier comprises one or more biodegradable, mucoadhesive polymeric carriers. In some embodiments, polymers such as polylactide-co-glycolide (PLGA), chitosan (for example in the form of chitosan nanoparticles, such as N-trimethyl chitosan (TMC)-based nanoparticles), alginate (such as sodium alginate) and carbopol are included. In some embodiments, the excipient or carrier comprises one or more hydrophilic polymers, such as sodium alginate or carbopol. In some embodiments, the vaccine comprises carbopol, for example in combination with starch. In some embodiments, the vaccine is formulated for intravenous or systemic administration. In some embodiments the vaccine comprises liposomes, immune-stimulating complexes (ISCOMs) and/or polymeric particles, such as virosomes.

In some embodiments, the carrier comprises a liquid solution, suspension, emulsion, tablet, pill, capsule, sustained release formulation, or powder. In some embodiments, the vaccine comprises a liquid, or a lyophilized or freeze-dried powder. In some embodiments, the vaccine is formulated as a suppository, with traditional binders and carriers such as triglycerides. In some embodiments, oral formulations include one or more standard carriers such as pharmaceutical grades of mannitol, lactose, starch, magnesium stearate, sodium saccharine, cellulose, and magnesium carbonate.

In some embodiments, the carrier comprises one or more biodegradable, mucoadhesive polymeric carriers. In some embodiments, polymers such as polylactide-co-glycolide (PLGA), chitosan, alginate and carbopol are included. In some embodiments, hydrophilic polymers, such as sodium alginate or carbopol, absorb to the mucus by forming hydrogen bonds, consequently enhancing nasal residence time, and in some embodiments are included in the disclosed vaccines.

In some embodiments, the vaccine is formulated as a particulate delivery system used for nasal administration or is formulated for intravenous or systemic administration or delivery. In some embodiments, the vaccine comprises liposomes, immune-stimulating complexes (ISCOMs) and/or polymeric particles, such as virosomes. In some embodiments, the liposome is surface-modified (e.g., glycol chitosan or oligomannose coated). In some embodiments, the liposome is fusogenic or cationic-fusogenic.

In some embodiments, the vaccine is lyophilized. In some embodiments, the disclosed vaccines are freeze-dried. In some embodiments the vaccine is vitrified in a sugar glass.

In some embodiments, the vaccine is formulated in a solvent or liquid such as a saline solution, a dry powder, or as a sugar glass. For example, in some embodiments, VLPs are used as vaccines by intranasal administration, or IM or ID injection, formulated in saline, dry powders or as sugar glasses made from trehalose, and/or are mixed with adjuvants to enhance the immune response to the vaccine. In some embodiments, the vaccine comprises a sugar glass. In some embodiments, the sugar glass comprises trehalose. In some embodiments, the vaccine comprises a VLP and an adjuvant embedded in the sugar glass. In some embodiments, the vaccine comprises VLPs or adjuvants formulated in salt buffered trehalose solutions that are printed and are dried. In some embodiments, the drying is by vitrification. In some embodiments, this provides the benefit of room temperature stability.

In some embodiments, the vaccine formulation contains trehalose and imiquimod. In some embodiments, the vaccine contains cyclodextrin such as sulfobutyl-β-cyclodextrin. In some embodiments, the vaccine antigen is embedded in a liposome formulation that comprises DOPC (1,2-dioleoyl-sn-glycero-3 -phosphocholine), DOPE (1,2-dioleoyl-sn-glycero-3-phosphoethanolamine), cholesterol and DSPE-peg2000 (1,2 distearoyl-sn-glycero-3-phophoethanoamine-N[amino(polyethelene glycol)-2000] (ammonium salt).

In some embodiments, the vaccine is formulated for microneedle administration. In some embodiments, the vaccine is formulated for intranasal, intradermal, intramuscular, topical, oral, subcutaneous, intraperitoneal, intravenous, or intrathecal administration. In some embodiments, the disclosed vaccines are formulated for intranasal administration, for example for mucosal immunization.

In some embodiments, the vaccine comprises a dose of 1 pg, 10 pg, 25 pg, 100 pg, 250 pg, 500 pg, 750 pg, 1 ng, 5 ng, 10 ng, 15 ng, 20 ng, 25 ng, 50 ng, 100 ng, 250 ng, 500 ng, 1 μg, 10 μg, 50 μg, 100 μg, 500 μg, 1 mg, 5 mg, 10 mg, 50 mg, 100 mg, 500 mg, or 1 g of the vaccine, or a range of doses defined by any two of the aforementioned doses. In some embodiments, the vaccine comprises a dose of 25 pL, 50 pL, 100 pL, 250 pL, 500 pL, 750 pL, 1 nL, 5 nL, 10 nL, 15 nL, 20 nL 25 nL, 50 nL, 100 nL, 250 nL, 500 nL, 1 μL, 10 μL, 50 μL, 100 μL, 500 μL, 1 mL, or 5 mL of the vaccine, or a range of doses defined by any two of the aforementioned doses. In some embodiments, the dose is on or in each microneedle of a microneedle device described herein.

### V. DEVICES

Disclosed herein, in certain embodiments, are microneedle devices comprising: a microneedle loaded with a vaccine as described herein. In some embodiments, the microneedle device comprises a substrate comprising a sheet and a plurality of microneedles extending therefrom. In some embodiments, each of said microneedles comprises a tip. In some embodiments, each of said microneedles comprises a base. In some embodiments, each of said microneedles comprises a hinge at the base connecting the microneedle to the sheet. In some embodiments, each of said microneedles comprises a well comprising the vaccine. In some embodiments, the vaccine is dehydrated. In some embodiments, the microneedle device comprises a sugar glass comprising the vaccine. In some embodiments, the sugar glass comprises trehalose. In some embodiments, the microneedle device comprises a vaccine patch such as a VaxiPatch.

In some embodiments, the microneedles comprise structures of micrometer to millimeter size. In some embodiments, the microneedles are designed to pierce the skin and deliver a vaccine to the epidermis or dermis of a subject. Microneedles offer some advantages over traditional sub-cutaneous or intramuscular injections. In some embodiments, microneedles are used to deliver the vaccine directly to the immune cells in the skin, which is advantageous for immunization purposes. The amount of vaccine needed for microneedle administration, compared to traditional sub-cutaneous or intramuscular injections, is smaller and can reduce production cost and time. In some embodiments, the microneedle is self-administered. In some embodiments, the vaccine is dried onto the microneedle, which greatly increases the stability of the vaccine at room temperature. Microneedle administration is painless, making it a more tolerated form of administration.

In some embodiments, microneedles are solid structures. In some embodiments, microneedles are hollow structures. In some embodiments, a vaccine is released through hollow structures (e.g., a liquid vaccine is injected or infused into the skin). In some embodiments, a vaccine is packaged onto a microneedle (for example, coated onto a surface of the microneedle after formation). In some embodiments, the vaccine is packaged onto a microneedle as a dried form. In some embodiments, the vaccine is dehydrated after being packaged onto a microneedle. In some embodiments, vaccines are packaged into a microneedle (for example, forming part of the microneedle itself, such as by deposition into the interior of the microneedle, or by inclusion in a mixture used to form the microneedle). In some embodiments, the vaccine is dissolved in the skin compartment. In some embodiments, the vaccine is injected into the skin. In some embodiments, microneedles are formed in an array comprising a plurality of microneedles. In some embodiments, the microneedle array is a 5×5 array of microneedles. In some embodiments, the microneedle array is physically or operably coupled to a solid support or substrate. In some embodiments, the solid support is a patch. In some embodiments, the microneedle array is applied directly to the skin for intradermal administration of a vaccine.

A microneedle array patch can be any suitable shape or size. In some embodiments, a microneedle array patch is shaped to mimic facial features, e.g., an eyebrow. In some embodiments, a microneedle array patch is the smallest size allowable to deliver a selected amount of bioactive agent.

The size and shape of the microneedles varies as desired. In some embodiments, microneedles include a cylindrical portion physically or operably coupled to a conical portion having a tip. In some embodiments, microneedles have an overall pyramidal shape or an overall conical shape. In some embodiments, the microneedle includes a base and a tip. In some embodiments, the tip has a radius that is less than or equal to about 1 micrometer. In some embodiments, the microneedles are of a length sufficient to penetrate the stratum corneum and pass into the epidermis or dermis. In certain embodiments, the microneedles have a length (from their tip to their base) between about 0.1 micrometer and about 5 millimeters in length, for instance about 5 millimeters or less, 4 millimeters or less, between about 1 millimeter and about 4 millimeters, between about 500 micrometers and about 1 millimeter, between about 10 micrometers and about 500 micrometers, between about 30 micrometers and about 200 micrometers, or between about 250 micrometers to about 1,500 micrometers. In some embodiments, the microneedles have a length (from their tip to their base) between about 400 micrometers to about 600 micrometers.

In some embodiments, the size of individual microneedles is optimized depending upon the desired targeting depth or the strength requirements of the needle to avoid breakage in a particular tissue type. In some embodiments, the cross-sectional dimension of a transdermal microneedle is between about 10 nm and 1 mm, or between about 1 micrometer and about 200 micrometers, or between about 10 micrometers and about 100 micrometers. In some embodiments, the outer diameter of a hollow needle is between about 10 micrometers and about 100 micrometers and the inner diameter of a hollow needle is between about 3 micrometers and about 80 micrometers.

In some embodiments, the microneedles are arranged in a pattern. In some embodiments, the microneedles are spaced apart in a uniform manner, such as in a rectangular or square grid or in concentric circles. In some embodiments, the microneedles are spaced on the periphery of the substrate, such as on the periphery of a rectangular grid. In some embodiments, the spacing depends on numerous factors, including height and width of the microneedles, the characteristics of a film to be applied to the surface of the microneedles, as well as the amount and type of a substance that is intended to be moved through the microneedles. In some embodiments, the arrangement of microneedles is a “tip-to-tip” spacing between microneedles of about 50 micrometers or more, about 100 micrometers to about 800 micrometers, or about 200 micrometers to about 600 micrometers.

In some embodiments, the microneedle comprises or consists of any suitable material. Example materials include metals, ceramics, semiconductors, organics, polymers, and composites. In some embodiments, materials of construction include, but are not limited to: pharmaceutical grade stainless steel, gold, titanium, nickel, iron, gold, tin, chromium, copper, alloys of these or other metals, silicon, silicon dioxide, and polymers. In some embodiments, the polymer is a biodegradable polymer or a non-biodegradable polymer. Representative biodegradable polymers include, but are not limited to: polymers of hydroxy acids such as lactic acid and glycolic acid polylactide, polyglycolide, polylactide-co-glycolide, and copolymers with PEG, polyanhydrides, poly(ortho)esters, polyurethanes, poly(butyric acid), poly(valeric acid), and poly(lactide-co-caprolactone). Representative non-biodegradable polymers include polycarbonate, polymethacrylic acid, ethylenevinyl acetate, polytetrafluorethylene and polyesters.

In some embodiments, the microneedle is dissolvable, biosoluble, biodegradable, or any combinations thereof “Biodegradable” is used to refer to any substance or object that is decomposed by bacteria or another living organism. Any suitable dissolvable, biosoluble, and/or biodegradable microneedles are contemplated for use with the vaccines and methods disclosed herein. In some embodiments, the dissolvable, biosoluble, or biodegradable microneedles are composed of water soluble materials. In some embodiments, these materials include chitosan, collagen, gelatin, maltose, dextrose, galactose, alginate, agarose, cellulose (such as carboxymethylcellulose or hydroxypropylcellulose), starch, hyaluronic acid, or any combinations thereof. In some embodiments, a selected material is resilient enough to allow for penetration of the skin. In some embodiments, the dissolvable microneedle dissolves in the skin within seconds, such as within about 5, 10, 15, 20, 25, 30, 45, 50, 60, 120, 180, or more seconds. In some embodiments, the dissolvable microneedle dissolves in the skin within minutes, such as within about 1, 2, 3, 4, 5, 6, 7, 8, 9, 10, 15, 20, 30, 60, 120 or more minutes. In some embodiments, the dissolvable microneedle comprises a dissolvable portion (such as the tip of the microneedle) and a non-dissolvable portion (such as the base of a microneedle), such that a portion of the microneedle structure dissolves in the skin. In some embodiments, the dissolvable microneedle encompasses the entire microneedle, such that the entire microneedle structure dissolves in the skin. In some embodiments, a dissolvable coating is formed on anon-dissolvable support structure such that only the coating dissolves in the skin. In some embodiments, the microneedle is coated with a polymer that is dissolvable, biodegradable, biosoluble, or any combinations thereof

In some embodiments, a vaccine is directly coated onto the dissolvable, biodegradable, or biosoluble microneedle. In some embodiments, a vaccine is contained within the dissolvable, biodegradable, or biosoluble microneedle itself (e.g., by forming part of the dissolvable polymer matrix). In some embodiments, a vaccine is mixed with a polymer matrix prior to molding and polymerization of microneedle structures.

In some embodiments, the microneedle array comprises a thin sheet of medical grade stainless steel (SS). In some embodiments, photochemical etching is used to create arrays in two dimensions (x, y axis). In some embodiments, each individual tip is formed and remains connected to the SS sheet by a pre-formed hinge. In some embodiments, the microneedles are formed with a sharp point and chiseled edges, and each has a pre-formed well designed to subsequently receive the appropriate vaccine. In some embodiments, a microfluidic dispensing instrument is used to deliver a precise amount of vaccine into each pre-formed well. In some embodiments, the microfluidic dispensing equipment simultaneously and/or accurately applies the fluid into hundreds of wells outlined on the stainless-steel sheet. In some embodiments, the small amount of vaccine dries immediately and adheres to the well of the microneedles. In some embodiments, the microneedle array comprises a 1.2 cm circular microarray of 37 microneedles. In some embodiments, the microneedles comprise photochemically etched stainless steel.

A variety of methods for manufacturing microneedles are available and any suitable method for manufacturing microneedles or microneedle arrays are contemplated for use with the vaccines and methods disclosed herein. In some embodiments, microneedles are manufactured using any suitable method, including, but not limited to: molding (e.g., self-molding, micromolding, microembossing, microinjection, and the like), casting (e.g., die-casting), or etching (e.g., soft microlithography techniques). In some embodiments, the microneedle device is prepared in accordance with Example 10. In some embodiments, the microneedle device is prepared in accordance with one or more steps described in Example 10.

### VI. KITS

Disclosed herein, in certain embodiments, are kits comprising: a vaccine as described herein, and comprising a microneedle loaded with the vaccine, a cleaning wipe, a desiccant, and a bandage. In certain embodiments the kit also contains a second adjuvant containing wipe where the adjuvant is imiquimod.

In some embodiments, the kit comprises containers or vials. In some embodiments, the containers or vials each contain a different VLP or vaccine. In some embodiments, the containers comprise VLPs in a suspension, such as with PBS or other pharmaceutically acceptable carrier. In some embodiments, the vaccine or VLPs are in a dried or powered form, such as lyophilized or freeze dried, configured to be reconstituted by an end user (for example with PBS or other pharmaceutically acceptable carrier). In some embodiments, the vaccine or VLPs are in trehalose sugar glasses for microneedle intradermal administration. In some embodiments, the kit comprises a first container comprising VLPs comprising a first antigen (e.g. a first HA subtype, or HA from a first influenza virus). In some embodiments, the kit comprises a second container comprising VLPs comprising a second antigen (e.g. a second HA subtype or HA from a second influenza virus). In some embodiments, the kit comprises a third container comprising VLPs comprising a third antigen (e.g. a first NA subtype). In some embodiments, the containers comprise a mixture of VLPs provided herein. In some embodiments, the containers in the kit comprise an adjuvant. In some embodiments, the adjuvant is in a separate container in the kit. In some embodiments, the containers comprise a pharmaceutically acceptable carrier such as PBS. In some embodiments, the pharmaceutically acceptable carrier is in a separate container (for example if the VLPs are freeze-dried or lyophilized). In some embodiments, the containers in the kit further comprise one or more stabilizers. In some embodiments, the kits comprise a device that permits administration of the VLPs to a subject. Examples of such devices include a microneedle in a VaxiPatch or other device provided herein. In some embodiments, the kit contains an imiquimod wipe.

### VII. MANUFACTURING METHODS

Disclosed herein, in certain embodiments, are methods of making a VLP (e.g. seVLP) comprising: microfluidically combining (i) a first solution comprising an antigen as described herein with (ii) a second solution comprising one or more lipids such as a first lipid and a second lipid. In some embodiments, the first and/or second solution comprises an aqueous solution. In some embodiments, the first and/or second solution comprises an ethanolic solution. In some embodiments, the antigen is bound to an anchor molecule. In some embodiments, the combining the first and second solutions, mixes the first and second solutions to form a VLP as described herein. In some embodiments, the VLP comprises a lipid vesicle as described herein. In some embodiments, the VLP comprises a lipid bilayer. In some embodiments, the lipid vesicle or the lipid bilayer comprises the first lipid and/or the second lipid with the anchor molecule embedded in the lipid bilayer.

In some embodiments, the method comprises: microfluidically combining (i) an aqueous solution comprising an antigen bound to an anchor molecule with (ii) an ethanolic solution comprising a first lipid and a second lipid, thereby mixing the aqueous solution with the ethanolic solution to form a VLP comprising a lipid bilayer comprising the first and second lipids with the anchor molecule embedded in the lipid bilayer. In some embodiments, microfluidically combining the aqueous solution with the ethanolic solution comprises mixing a stream of the aqueous solution with a stream of the ethanolic solution.

In some embodiments, the method comprises: providing an aqueous solution comprising a peptide comprising an antigen domain and a membrane anchor domain; providing an ethanolic solution comprising a first lipid and a second lipid; and/or combining the aqueous solution with the ethanolic solution to produce a VLP wherein the peptide is anchored to the lipid vesicle by the membrane anchor domain with the antigen domain on an outward surface of the lipid vesicle. In some embodiments, combining the aqueous solution with the ethanolic solution comprises microfluidic mixing of a stream of the aqueous solution with a stream of the ethanolic solution.

In some embodiments, the antigens are produced from purified proteins produced using recombinant DNA methods. In some embodiments, defined purified recombinant proteins are mixed with defined lipids using a microfluidic mixer to form chemically defined VLPs (e.g. seVLPs or smVLPs). An example of a microfluidic mixer is a NanoAssmblr (Precision Nanosystems, Inc.). In some embodiments, the VLPs (e.g. seVLPs) are produced by: (1) producing essentially pure antigenic proteins in any recombinant DNA-based protein expression system (2) chemically defined lipids, and (3) assembled in vitro using a microfluidic mixer.

In some embodiments, the method produces seVLPs by a controlled microfluidics process. In some embodiments, the microfluidics produce liposomes of uniform size in scalable commercial quantities. In some embodiments, the microfluidics use mild solvents that preserve the native properties of the antigens. In some embodiments, the seVLPs are produced without the use of dialysis or a detergent. In some embodiments, the seVLPs are produced with dialysis or a detergent.

In some embodiments, the antigen is purified using a detergent such as a detergent described herein. In some embodiments, the detergent is cleavable. In some embodiments, the detergent-purified antigen is used to make a VLP. In some embodiments, the detergent comprises octyl glucoside (n-octyl-β-d-glucoside). In some embodiments, cleavable detergent reduces the time in manufacturing to remove the detergents (for example, from about 5 days to minutes). In some embodiments, the detergent comprises a chemically cleavable detergent (CCD). In some embodiments, the CCD is derived by disulfide incorporation of a disulfide bond in a detergent such as n-dodecyl-β-D-maltopyranoside. In some embodiments, the disulfide bond of the detergent is cleaved by tris(2-carboxyethyl)phohine (TCEP). In some embodiments, the disulfide bond of the detergent is cleaved under conditions that do not cleave disulfides in native proteins that contain disulfide bonds. Some embodiments include a cleavable disulfide edition of octyl glucoside.

In some embodiments, the VLPs (e.g. seVLP) are made by two steps. In the first step the antigen is produced and/or purified by recombinant DNA methods. Second, the antigen is mixed with defined lipids by microfluidics. In some embodiments, an antigen is expressed in a protein expression system. In some embodiments, the antigen is HA, NA, or an influenza matrix protein (such as influenza M1 or M2). In some embodiments, protein expression system is bacterial, yeast, plant, insect cell or mammalian cell based. In some embodiments, these cells are transfected or infected with (1) a virus encoding an antigen or a virus encoding an antigen, and in some embodiments, also with (2) a virus encoding an antigen, under conditions sufficient to allow for expression of the antigen in the cell. Second, in some embodiments, the antigens are mixed with DOPC, DOPE and cholesterol in a microfluidizer such as the Nanoassemblr™ Benchtop (Precision Nanosystems, Inc., Vancouver, Canada). In some embodiments, the seVLPs are made by extrusion. In some embodiments, the extrusion comprises the use of an extruder device such as an extruder device from Avanti Polar Lipids.

In some embodiments, seVLPs are formed with their antigens in an aqueous solution, and with lipids in an ethanolic solution. Two streams, each containing either the aqueous or ethanolic solution, are combined by microfluidic mixing in a mixer such as a Nanoassemblr™ Benchtop (Precision Nanosystems, Inc., Vancouver, Canada) from Precision NanoSystems. In some embodiments, the VLP comprises a lipid component that contains or comprises at least one synthetic or essentially pure phosphatidylcholine (PC) species and at least one synthetic or essentially pure phosphatidylethanolamine (PE) at a molar ratio of, 3:1 to 1:3, characterized in that the acyl chains have between 4 and 18 carbon atoms, the total number of unsaturated bonds in the acyl chains being four or less. In some embodiments, synthetic 1,2-dioleoyl-sn-glycero-3-phosphocholine (DOPC) and synthetic 1,2-choleoyl-S7-glycero-3-phosphoetanolamine (DOPE) are used. In some embodiments, DSPE-peg2000 (1,2 distearoyl-sn-glycero-3-phophoethanoamine-N[amino(polyethelene glycol)-2000] (ammonium salt), or a related lipid, is used (for example, mixed with a purified antigen) to make the VLP. In some embodiments, the lipid component is supplemented with sterol such as cholesterol, or with a sterol derivative at a ratio of 0-30 mol % of total added phospholipid. In some embodiments, the VLPs are made with, and comprise or consist of synthetic or essentially pure components. Some embodiments include an exogenously added, non-viral phospholipid species of defined quality, purity and chemical structure. Some embodiments include synthetic or essentially pure PC and/or PE species. In some embodiments, the VLP is made by combining DOPC, DOPE, cholesterol, and DSPE-peg2000.

In some embodiments, the VLPs are produced with a ratio of DOPE to DOPC between 4:1 and 0.5:1. In some embodiments, a sterol or sterol derivative is added to increase the storage stability of the seVLPs. Examples of sterol derivatives include cholesterol, cholesterol hemisuccinate, phytosterols such as lanosterol, ergosterol, and vitamin D and vitamin D related compounds. In some embodiments, the amount of cholesterol to DOPC and DOPE combined is about 20 mol %.

Some embodiments include a predetermined ratio of antigen to lipids. A distinguishing feature of some embodiments of this disclosure is the insertion of the antigen into the membrane of the seVLP during the microfluidic mixing. To prepare seVLPs, a Nanoassemblr™ Benchtop (Precision Nanosystems, Inc., Vancouver, Canada) is used with a 300 p.m Staggered Herringbone Micromixer. In some embodiments, the lipids are dissolved at a predetermined ratio in methanol or ethanol, and the antigen is dissolved in PBS, 10 mM, pH 7.4 aqueous buffer containing 0.1-10% octyl glucoside (n-octyl-β-d-glucoside) (OG), a detergent. Another detergent is 1,2-dicaproyl-sn-glycero-3-phosocholine (DCPC). In some embodiments, the antigens with transmembrane domains are kept in detergent(s) prior to forming seVLPs. In some embodiments, a critical micelle concentration (c.m.c.) of OG and DCPC is 25 mM and 14 mM respectively. In some embodiments, a c.m.c. below 5mM is used to remove the detergent by dialysis. As an example, influenza rHA protein in aqueous buffered saline and 15 — 20 mM DCPC is mixed with DOPE, DOPC, cholesterol and 2-5 mM DCPC in ethanol with the Nanoassemblr™ Benchtop such that the eluant is slightly below the 14 mM c.m.c. of DSPC. In some embodiments, this fast detergent removal leads to simultaneous coalescence of lipid-detergent and lipid-protein detergent micelles resulting in direct co-reconstitution of lipids and proteins forming homogeneous seVLPs. In some embodiments, without detergent, the transmembrane domains of antigens form aggregates, which in the case of influenza HA, leads to rosette formation. In some embodiments, such aggregation is irreversible. In some embodiments, seVLPs comprise200-500 nmol DOPC, 600-1000 nmol DOPE, about and 200-300 nmol cholesterol per mg of recombinant influenza membrane protein(s). The flow rate ratio between the aqueous and solvent stream is between 1:1 to 5:1 (aqueous:alcohol) with a 3:1 ratio preferred. The total flow rate is 1-10 mL/min. seVLPs were purified and concentrated using 750 kD tangential flow (TFF) column Spectra/Por® Dialysis membrane, Biotech CE Tubing, Spectrum Laboratories, USA).

In some embodiments, the VLP (e.g. seVLP) has a narrow size distribution. In some embodiments, lipid vesicles or VLPs have a diameter (particle size) in the range of 40 to 200 nm, from 50 nm to 150 nm, or from 70 nm to 130 nm. In some embodiments, the lipid vesicles or VLPs have a homogeneous size distribution with less than 15% or 10% of the VLPs having a particle size above 150 nm, and less than 15% or 10% below 50 nm. In some embodiments, the modal diameter is below 90 nm. In some embodiments, cholesterol lowers the need for DOPC and stabilizes the seVLPs.

In some embodiments, microfluidic preparation of the VLPs is used. In some embodiments, the VLPs are not prepared by sonication, and/or or detergent removal is not performed by dialysis. In some embodiments, microfluidic preparation of the VLPs tightens the size variation to a more uniform size compared to VLPs such as eVLPs prepared by sonication or detergent removal by dialysis.

Some VLPs are made without the use of a detergent in one or more steps, or in all of the steps, of the method. In some embodiments, the VLP (e.g. smVLP) is produced with polymer based nanodiscs. In some embodiments, a smVLP is made by a method that includes the use of a polymethacrylate (PMA) copolymer. In some embodiments, the methacrylate copolymer is made to mimic the amphipathic helical structure of a natural apolipoprotein that forms a lipid bilayer nanodisc. In some embodiments, amphipathic a-helical peptides are used to form nanodiscs. In some embodiments, an amphipathic structure of these proteins and peptides is beneficial to form lipid nanodiscs. In some embodiments, to mimic the amphiphilic nature of such proteins or peptides, amphiphilic polymethacrylate random copolymers comprising hydrophobic and hydrophilic side chains are used to produce a nanodisc-forming polymer. In some embodiments, their monomer sequence is random, but the amphiphilic polymethacrylate random copolymer provides an amphiphipathic structure upon its interaction with a lipid bilayer. In some embodiments, hydrophobic butyl methacrylate and cationic methacroylcholine chloride of the resultant polymer interact with hydrophobic acyl chains and anionic phosphate headgroups of lipids, respectively, to form a lipid nanodisc formation surrounded by the polymer. In some embodiments, the copolymers are synthesized using free radical polymerization initiated by azobis(isobutyronitrile) (AIBN). In some embodiments, the molecular weight of a polymer is adjusted by varying the amount of methyl 3-mercaptopropionate used as a chain-transfer agent. In some embodiments, the hydrophobic/cationic ratio is varied by the feed ratio of two monomers. In some embodiments, the resultant polymer is purified by reprecipitation in diethyl ether, which in some embodiments provides the benefit of complete removal of unreacted monomers.

In some embodiments, the ability of each synthesized polymer to solubilize lipids is examined by carrying out turbidity measurements on large unilamellar vesicles of DMPC (1,2-dimyristoyl-sn-glycero-3-phosphocholine) prepared by the extrusion method (LUVs of 100 nm in diameter). In some embodiments, the addition of a polymer to DMPC vesicles results in a decrease of the solution turbidity in many cases, reflecting polymer-induced fragmentation of vesicles and resulting lipid nanodisc formation. In some embodiments, an optimization of the amphiphilic balance is beneficial to obtain efficient nanodisc-forming polymers.

In some embodiments, nanodiscs comprise or are formed using styrene maleic acid (SMA) polymers or co-polymers. In some embodiments, addition of the SMA to a synthetic or biological lipid membrane leads to the spontaneous formation of nanodiscs. In some embodiments, such polymer-bounded nanodiscs comprise a bilayer organization of incorporated lipid molecules that is conserved. In some embodiments, an advantage of using SMA is the ability of the SMA polymer to directly extract proteins from a native cell membrane environment. Depending on the origin of the lipid material, the terms SMALPs is used in some embodiments for particles derived from synthetic liposomes and synthetic natural nanodiscs is used in some embodiments to refer to isolations from biological membranes. In some embodiments, the use of SMALPs comprises the isolation of a membrane protein with detergents, insertion of the membrane protein into a liposome and then the formation of the nanodisc with the addition of SMA. In some embodiments, this has the advantage that the lipids are defined in vitro. In some embodiments, a native nanodisc system combines a solubilizing power similar to detergents with the small particle size of nanodiscs, while conserving a minimally perturbed native lipid environment that stabilizes the protein.

In some embodiments, SMALPs are made of poly(styrene-co-maleic acid) (SMA). In some embodiments, the SMA is incorporated into membranes and spontaneously forms SMALPs. In some embodiments, a Styrene Maleic Anhydride Co-polymer reagent uses a styrene to maleic acid ratio of 2:1. In some embodiments, an anhydride polymer powder is obtained and converted to an acid using hydrolysis. In some embodiments, The Styrene Maleic Anhydride Co-polymer is dissolved in 1 M NaOH. In some embodiments, the reaction is carried out while heating and refluxing a solution. In some embodiments, after cooling at room temperature. In some embodiments, the Styrene Maleic Anhydride Co-polymer is precipitated by reducing the pH to below 5 by the addition of concentrated HCl. In some embodiments, the precipitate is washed three times with water followed by separation using centrifugation. In some embodiments, at the end of the third wash the precipitate is resuspended in 0.6 M NaOH. In some embodiments, the solution is precipitated and washed again, and resuspended in 0.6 M NaOH. In some embodiments, the pH is then adjusted to pH 8. In some embodiments, the polymer is lyophilized. In some embodiments, the Styrene Maleic Anhydride Co-polymer is added to a suspension of lipid. In some embodiments, the SMA interacts with the lipid bilayer, self-assembling into SMALPs.

In some embodiments, when used as VLPs presenting antigens to the immune system the nanodisc technology provides a spectrum of membrane VLPs (mVLPs) of mVLPs (from natural mVLPs derived from cells, to semi-synthetic semi-synthetic mVLPS where exogenous lipids are supplemented to the lipid mix to fully smVLPs where all the lipids are defined and supplied in vitro.

In some embodiments, DIBMA or SMA provides the ability of directly extracting membrane proteins from native cell membranes. In some embodiments, (e.g. when VLPs described herein comprise influenza HA, NA or M2 antigens produced by recombinant DNA methods), this simplifies vaccine nanodisc formation. In some embodiments, DIBMA is directly added to cell membranes to extract vaccine antigen(s) produced by recombinant methods that are embedded in the membrane of the protein expression system. In some embodiments, DIBMA is obtained from Anatace. In some embodiments, the antigen of a nanodisc comprising DIBMA comprises a HIS tag at, for example, the C-terminus of the antigen. In some embodiments, the antigen and/or the DIBMA nanodiscs are purified by IMAC chromatography. In some embodiments, the nanodiscs comprise an antigen (e.g. HA) embedded in a flat lipid membrane of the producer cell defined by a belt of DIBMA In some embodiments, DIBMA is supplemented with DMPC (1,2-dimyristoyl-sn-glycero-3-phosphocholine). In some embodiments, such supplementation provides the benefit of improving extraction of the antigen from the producer cells. In some embodiments, the VLPs comprise native nanodiscs. In some embodiments, the nanodiscs are synthetic or semi-synthetic.

In some embodiments, a vector is included that comprises a nucleic acid molecule encoding an antigen comprising a recombinant peptide. In some embodiments, the vector is any suitable vector for expression of the recombinant polypeptide, such as a mammalian expression vector. In some embodiments, the vector is the pCAGGS expression vector or the pFastBacl baculovirus transfer vector plasmid. In some embodiments, any expression vector used for transfection or baculovirus expression is used. In some embodiments, the vector comprises a promoter operably linked to the nucleic acid sequence encoding the recombinant peptide. In particular examples, the promoter is a CMV or SV40 promoter.

A. Anti₂en Generation in Mammalian Cells

Antigens for use with the vaccines and methods described herein are made by any suitable method. In some embodiments, a nucleic acid molecule encoding a desired antigen such as a HA protein or NA protein, in some embodiments, along with a nucleic acid molecule encoding an influenza matrix protein(s), are each cloned into an expression plasmid (e.g., pCAGGS). In some embodiments, the antigen, M1, M2, NA and/or HA coding sequences is codon-optimized for expression in mammalian cells. In some embodiments, a resulting vector is transfected into cells, along with the matrix protein(s) containing vector. In some embodiments, matrix protein(s) are expressed from the same vector as HA or NA. In some embodiments, the transfection is a transient transfection. In some embodiments, the cells include 293 cells, Vero cells, A549 cells, CHO cells, or the like.

In some embodiments, the cells are incubated under conditions that allow the antigen to be expressed by the cell. In some embodiments, the mammalian cells are incubated for about 72 hours at 37 degree C. In some embodiments, proteins are purified by standard techniques well known to those in the art.

In some embodiments, the amounts of proteins are determined by western blot or other quantitative immunoassay, Bradford assay, and in the case of HA the FDA approved potency test, the single radial immunoassay (SRID) test.

B. Antigen Generation in Insect Cells

In some embodiments, the antigen is produced in an insect cell. In some embodiments, a nucleic acid molecule encoding an antigen. In some embodiments, along with a nucleic acid molecule encoding an influenza matrix protein(s), are each cloned into a baculovirus transfer vector plasmid (e.g., pFastBacl, Invitrogen, Carlsbad, Calif). In some embodiments, the matrix protein(s) are expressed from the same baculovirus transfer vector as HA or NA. In some embodiments, expression of the antigen, HA, NA, M1 and/or M2 is under the transcriptional control of the Autographa californica multiple nuclear polyhedrosis virus (AcMNPV) polyhedrin promoter. In some embodiments, the antigen, M1, M2, NA and/or HA coding sequences is codon-optimized for expression in insect cells. In some embodiments, each recombinant baculovirus construct is plaque purified and master seed stocks prepared, characterized for identity, and used to prepare working virus stocks. In some embodiments, titers of baculovirus master and working stocks are determined by using a rapid titration kit (e.g., BacPak Baculovirus Rapid Titer Kit; Clontech, Mountain View, Calif.).

In some embodiments, insect cells, such as S. frugiperda Sf9 insect cells (ATCC CRL-1711), are maintained as suspension cultures in insect serum free medium (e.g., HyQ-SFX HyClone, Logan, Utah) at 27±2° C. In some embodiments, recombinant baculovirus stocks are prepared by infecting cells at a low multiplicity of infection (MOI) of <0.01 plaque forming units (pfu) per cell and harvested at 68-72 h post infection (hpi).

In some embodiments, a resulting antigen-containing baculovirus vector is used to infect cells. In some embodiments, along with the matrix protein(s) containing baculovirus vector. In some embodiments, about 2-3x10⁶ cells/ml are infected with the antigen-containing baculovirus vector. The resulting infected cells are incubated with continuous agitation at 27±2° C. and harvested about 68-72 hpi, for example by centrifugation (e.g., 4000.times.g for 15 minutes). In some embodiments, the antigen is purified by a standard method known in the art.

### VIII. METHODS OF USE

Disclosed herein, in certain embodiments, are methods of preventing, reducing the occurrence of, and/or reducing the severity of a disease comprising: administering a vaccine as described herein to a subject in need thereof. Disclosed herein, in certain embodiments, are methods of preventing a disease comprising: administering a vaccine as described herein to a subject in need thereof. Disclosed herein, in certain embodiments, are methods of reducing the occurrence of a disease comprising: administering a vaccine as described herein to a subject in need there. Disclosed herein, in certain embodiments, are methods of reducing the severity of a disease comprising: administering a vaccine as described herein to a subject in need thereof

In some embodiments, the method comprises administering a VLP (e.g. seVLP or smVLP) as described herein to a subject. In some embodiments, the administration prevents the severity of the disease. In some embodiments, the administration reduces the occurrence of the disease. In some embodiments, the administration reduces the severity of the disease. In some embodiments, the administration prevents, reduces the occurrence of, and/or reduces the severity of the disease. In some embodiments, the method comprises preventing, reducing the occurrence of, or reducing the severity of a disease. In some embodiments, the method comprises administering the vaccine as described herein to a subject; wherein the administration prevents, reduces the occurrence of, or reduces the severity of the disease.

In some embodiments of the method, the disease is an infection. In some embodiments, the disease comprises a bacterial, fungal, or viral infection. In some embodiments, the viral infection comprises an influenza infection. In some embodiments, the subject is a mammal or human subject.

Disclosed herein, in certain embodiments, are methods for preventing, reducing the occurrence of, or reducing the severity of a disease comprising: administering the vaccine to a subject; wherein the administration prevents, reduces the occurrence of, or reduces the severity of the disease. In some embodiments, the disease is an infection. In some embodiments, the disease is a bacterial, fungal, or viral infection. In some embodiments, the viral infection is an influenza infection. In some embodiments, the subject is a mammal or human subject.

In some embodiments, the administration comprises administration by one or more needles or microneedles. In some embodiments, the administration comprises administration by a pre-formed liquid syringe. In some embodiments, the administration comprises intranasal, intradermal, intramuscular, skin patch, topical, oral, subcutaneous, intraperitoneal, intravenous, or intrathecal administration. In some embodiments, the administration comprises administering a dose of 1 pg, 10 pg, 25 pg, 100 pg, 250 pg, 500 pg, 750 pg, 1 ng, 5 ng, 10 ng, 15 ng, 20 ng, 25 ng, 50 ng, 100 ng, 250 ng, 500 ng, 1μg, 10 μg, 50 μg, 100 μg, 500 μg, 1 mg, 5 mg, 10 mg, 50 mg, 100 mg, 500 mg, or 1 g of the vaccine, or a range of doses defined by any two of the aforementioned doses. In some embodiments, 100 pL-20 nL of the vaccine is administered by each microneedle. In some embodiments, 5-20 nL of the vaccine is administered by each microneedle. In some embodiments, 10-20 nL of the vaccine is administered by each microneedle.

A. Methods of Administration

Any of the disclosed vaccines are administered to a subject by any suitable method. Suitable methods of administration include, but are not limited to, intradermal, intramuscular, intraperitoneal, parenteral, intravenous, systemic, subcutaneous, mucosal, vaginal, rectal, intranasal, inhalation or oral. In some embodiments, parenteral administration, such as subcutaneous, intravenous or intramuscular administration, is achieved by injection. In some embodiments, injectables are prepared in conventional forms, either as liquid solutions or suspensions, solid forms suitable for solution or suspension in liquid prior to injection, or as emulsions. In some embodiments, injection solutions and suspensions are prepared from sterile powders, granules, tablets, and the like. In some embodiments, the administration is systemic. In some embodiments, the administration is local. In some embodiments, the vaccines provided herein are formulated for mucosal vaccination, such as oral, intranasal, pulmonary, rectal and vaginal. In a specific example, this is achieved by intranasal administration. In some embodiments, the administration comprises administering a vaccine as described herein comprising a sugar glass. In some embodiments, the sugar glass comprises trehalose.

In some embodiments, the administration comprises administration by a pre-formed liquid syringe. In some embodiments, the administration comprises administration by one or more needles or microneedles. In some embodiments, 100 pL-20 nL of the vaccine is administered by each microneedle. In some embodiments, the administration comprises intranasal, intradermal, intramuscular, skin patch, topical, oral, subcutaneous, intraperitoneal, intravenous, systemic, or intrathecal administration.

In some embodiments, the administration comprises rubbing or wiping a subject's skin with a wipe at a site of administration prior to injecting the vaccine with a needle or microneedle. In some embodiments, the wipe is a cleaning wipe. In some embodiments, the wipe is an imiquimod wipe. In some embodiments, the imiquimod wipe is rubbed into a subject's skin at the subject's site of administration such that the imiquimod is rubbed into the skin at the site be vaccinated prior to injecting the vaccine into the site of administration with a microneedle device.

Some embodiments include microneedle administration. Some embodiments include skin patch administration. Some embodiments include microneedle skin patch administration. In some embodiments, microneedles are placed on cleaned skin of the subject and pressed into the skin. In some embodiments, the microneedle skin patch comprises a dose of vaccine loaded on or in the microneedles in a liquid dispensing step. In some embodiments, microfluidic dispensing of 10-20 nL per microneedle is used.

In some embodiments, the vaccines are dried in a well inside each microneedle. In some embodiments, this keeps the microneedles sharp enough for a light force of under 10 Newtons to be successful in delivery. In some embodiments, the vaccines are dried outside each microneedle. In some embodiments, a microneedle array is used for administration.

In some embodiments, vaccines are packaged onto microneedles. In some embodiments, vaccines are packaged or embedded into microneedles. In some embodiments, the vaccine is dehydrated after packaging into or onto the microneedle. In some embodiments, the microneedle is packaged individually at a unit dose of vaccine. In some embodiments, the unit dose is effective in inducing an immune response in a subject to the antigen. In some embodiments, the unit dose is effective in inducing an immune response in a subject to the antigen after storage for at least about one week (e.g., about or more than about 1, 2, 3, 4, 6, 8, 12, or more weeks) at room temperature. In some embodiments, the unit dose is effective in inducing an immune response in a subject to the antigen after storage for at least about one month (e.g., about or more than about 1, 2, 3, 4, 5, 6, 8, 10, 12, or more months) at room temperature. In some embodiments, the vaccine is present in an amount effective to induce an immune response in the subject to the antigen. In some embodiments, the microneedle administration is painless.

In some embodiments, the vaccine antigen is expressed in terms of an amount of antigen per dose. In some embodiments, a dose has 100 μg antigen or total protein (e.g., from 1-100 μg, such as about 1μg, 5μg, 10 μg, 25 μg, 50 μg, 75 μg or 100 μg). In some embodiments, expression is seen at much lower levels (e.g., 1 μg/dose, 100 ng/dose, 10 ng/dose, or 1 ng/dose).

In some embodiments, the subject is pre-treated with an adjuvant before vaccination. In some embodiments, the adjuvant is imiquimod.

B. Timing of Administration

In some embodiments, the method comprises multiple administrations or doses of a vaccine as described herein. In some embodiments, a disclosed vaccine is administered as a single or as multiple doses (e.g., boosters). In some embodiments, the first administration is followed by a second administration. In some embodiments, the second administration is with the same, or with a different vaccine than the vaccine administered. In some embodiments, the second administration is with the same vaccine as the first vaccine administered. In some embodiments, the second administration is with a vaccine comprising a different VLP (e.g. seVLP or smVLP) than the first vaccine administered. In some embodiments, if the first vaccine includes a first HA subtype and a second HA subtype, the second vaccine comprises a third HA subtype and a fourth HA subtype, wherein all four subtypes are different (such as four of H1, H2, H3, H5, H7, and H9).

In some embodiments, the vaccines containing two or more VLPs are administered as multiple doses, such as 2, 3, 4, 5, 6, 7, 8, 9, or 10 doses (such as 2-3 doses). In some embodiments, the timing between the doses is at least 1 week, at least 2 weeks, at least 3 weeks, at least 4 weeks, at least 6 weeks, at least 8 weeks, at least 12 weeks, at least 2 months, at least 3 months, at least 4 months, at least 5 months, at least 6 months, at least 1 year, at least 2 years, or at least 5 years, such as 1-4 weeks, 2-3 weeks, 1-6 months, 2-4 months, 1 week, 2 weeks, 3 weeks, 4 weeks, 5 weeks, 6 weeks, 7 weeks, 8 weeks, 9 weeks, 10 weeks, 12 weeks, 1 month, 2 months, 3, months, 4, months, 5 months, 6 months, 1 year, 2 years, 5 years, or 10 years, or combinations thereof (such as where there are at least three administrations, wherein the timing between the first and second, and second and third doses, are in some embodiments the same or different).

C. Dosages

In some embodiments, the method comprises administering a dose of 1 pg, 10 pg, 25 pg, 100 pg, 250 pg, 500 pg, 750 pg, 1 ng, 5 ng, 10 ng, 15 ng, 20 ng, 25 ng, 50 ng, 100 ng, 250 ng, 500 ng, 1 μg, 10 μg, 50 μg, 100 μg, 500 μg, 1 mg, 5 mg, 10 mg, 50 mg, 100 mg, 500 mg, or 1 g of the vaccine or VLP (e.g. seVLP or smVLP), or a range of doses defined by any two of the aforementioned doses.

In some embodiments, the subject is administered (e.g., intravenous or systemic) about 1 to about 100 μg of each VLP, such as about 1 μg to about 50 μg, 1 μg to about 25 μg, 1 μg to about 5 μg, about 5 μg to about 20 μg, or about 10 μg to about 15 μg of each VLP. In some embodiments, the subject is administered about 15 μg of each VLP. In some embodiments, the subject is administered about 10 μg of each VLP. In some embodiments, the subject is administered about 20 μg of each VLP. In some embodiments, the subject is administered about 1 μg or 2 μg of each VLP.

In some embodiments, the dose administered to a subject is sufficient to induce a beneficial therapeutic response in the subject over time, or to inhibit or prevent an infection. In some embodiments, the dose varies from subject to subject, or is administered depending on the species, age, weight and general condition of the subject, the severity of an infection being treated, and/or the particular vaccine being used and its mode of administration.

D. Methods for Measuring Immune Response

Some embodiments include measuring an immune response. Some embodiments include a method for determining whether a vaccine disclosed herein elicits or stimulates an immune response, such as achieve a successful immunization. Although exemplary assays are provided herein, the disclosure is not limited to the use of specific assays.

In some embodiments, following administration of a vaccine provided herein, one or more assays are performed to assess the resulting immune response. In some embodiments, the assays are also performed prior to administration of a vaccine, and/or to serve as a baseline or control. In some embodiments, samples are collected from the subject following administration of the vaccine, such as a blood or serum sample. In some embodiments, the sample is collected at least 1 week, at least 2 weeks, at least 3 weeks, at least 4 weeks, at least 5 weeks, at least 6 weeks, or at least 8 weeks (such as 1, 2, 3, 4, 5, 6, 7, 8, 9, 10, 11, or 12 weeks) after the first vaccine administration. In some embodiments, subsequent samples are obtained as well, for example following subsequent vaccine administrations.

1. Hemagglutination Titer Assay

In some embodiments, following production and purification of a vaccine provided herein, a hemagglutination titer assay is performed. In some embodiments, such assays are performed to measure or evaluate hemagglutinating units (HAU). In some embodiments, this is used to evaluate that the VLP (e.g. seVLP or smVLP) presents functional HA trimers and is in some embodiments used to quantify HA protein in the VLP preparation. Hemagglutination titers are also used to quantify the amount of influenza virus used a challenge virus, or for example to quantify amount of virus (titering) present in the lungs or respiratory tract of challenged animals. In some embodiments, vaccinated subjects show a reduction in viral titers as compared to mock-vaccinated subjects.

In some embodiments, the assay is used to quantify the amount of VLP or also to quantify virus in a sample, such as a lung sample from a virus challenged subject previously administered a vaccine provided herein. In some embodiments, vaccine is serially diluted (e.g., 2-fold from 1:4 to 1:4096) and then added to wells containing red blood cells (RBCs). In some embodiments, the RBC solution (such as 0.75% to 1% RBC) is added to the wells. In some embodiments, the mixture is then incubated for 30 min at room temperature, which allows the RBC to settle. In some embodiments, the samples are then analyzed for their resulting agglutination pattern, for example by examining microtiter wells in which the sample is placed. For example, in a microtiter plate placed on its edge, the RBC in the RBC control wells will flow into a characteristic teardrop shape (no influenza virus is present so there is no agglutination). In some embodiments, wells that contain influenza virus agglutinate the RBC to varying degrees. In some embodiments, the wells with the greatest amount of virus will appear cloudy, because the virus has cross-linked red blood cells, preventing their pelleting. In some embodiments, lesser amounts of virus in succeeding wells result in partial agglutination, but the pellet will not stream into a teardrop shape similar to the pellets in the RBC control wells. In some embodiments, the endpoint is determined as the greatest dilution of the vaccine resulting in complete agglutination of the RBC.

In some embodiments, a number of hemagglutinating units (HAU) in the sample being titered is determined. The HA titer is the reciprocal of the dilution of the last well of a series showing complete agglutination of the RBC (e.g., if the last dilution is 1:640, the titer of the sample is 640 HA units/5 μl sample).

2. Hemagglutination Inhibition (HA1) Assay

In some embodiments, following administration of a vaccine provided herein, a hemagglutination inhibition (HA1), assay is performed. In some embodiments, influenza viruses agglutinate red blood cells, a process called hemagglutination. In some embodiments, in the presence of specific antibody to the surface hemagglutinin, hemagglutination is blocked. In some embodiments, this phenomenon provides the basis for the HA1 assay, which is used to detect and quantitate specific antiviral antibodies in serum. Thus, HA1 measures the presence of antibodies that block HA receptor binding (as assessed by hemagglutination of RBC).

In some embodiments, sera to be evaluated for the presence of antibodies against the head of hemagglutinin is treated with receptor destroying enzyme (RDE) at 37° C. overnight. In some embodiments, the following day, RDE is inactivated by incubation at 56° C. for 1 hour. In some embodiments, assay plates used are 96-well, nonsterile, non-tissue culture-treated, round-bottom microtiter plates. In some embodiments, two-fold serial dilutions are carried out on each sample down the plate from row B through row G. 50 μl of working dilution of viral antigen (a set number of HAU) is added to all wells of the microtiter plates except for row H (the RBC control wells) and the antigen control wells. In some embodiments, the plates are incubated for 30 min at room temperature. 50 μl 1% RBC suspension in PBS is added to all wells and the plates incubated for 30 to 45 min at room temperature. In some embodiments, the microtiter plate is analyzed to read the agglutination patterns. In some embodiments, the negative control wells (those containing normal serum without anti-influenza antibodies) will appear cloudy, because the influenza virus has completely agglutinated the RBC. In some embodiments, the positive control wells (those containing known anti-influenza antiserum) will have RBC pellets similar in appearance to the row H control pellets as long as there is sufficient anti-influenza antibody to inhibit agglutination. In some embodiments, with increasing serum dilution, the amount of antibody will decrease so that increasing amounts of RBC agglutination become apparent. In some embodiments, the hemagglutination inhibition (HA1) titer for each serum sample is the reciprocal of the greatest dilution which completely inhibits the agglutination of the RBC (e.g., the last well in a dilution series forming an RBC pellet). In some embodiments, the HA1 titer for each sample is the mean of the endpoint titers of its duplicate dilution series. In some embodiments, if the titer of the duplicates differs by more than one two-fold dilution, the HA1 titer is repeated for that sample.

3. Influenza Virus Neutralization Assay

In some embodiments, following administration of a vaccine provided herein, a neutralization assay is performed. In some embodiments, serum samples from subjects who received a vaccine provided herein are diluted, influenza virus is added, and the amount of serum necessary to prevent virus growth determined. In some embodiments, neutralization assesses the presence of antibodies that inhibit viral replication. In some embodiments, antibodies to the stalk of HA, for example, neutralize viral replication but not affect hemagglutination because the epitope is not around the receptor binding domain. In some embodiments, antibodies that bind to the head and inhibit hemagglutination are usually neutralizing.

In some embodiments, the serum samples are incubated in tissue culture medium (such as DMEM/5% FBS containing antibiotics), for example in 96-well, round-bottom, tissue culture-treated microtiter plate. In some embodiments, the serum samples are serially diluted, for example in duplicate adjacent wells of a microwell plate (for example initially diluted 1:10 to a dilution of the sample of 1:640). In some embodiments, previously titered influenza virus (of any subtype) are diluted to contain 1 TCID_{50/50} μl. In some embodiments, equal amounts of the working stock virus (such as about 50 TCID₅₀) are added to each serum sample (comprising the serial dilutions), and incubate at 37° C. for 1 hr. In some embodiments, with this protocol, the same neutralization titer is obtained if the final amount of virus is between 10 to 100 TCID₅₀. In some embodiments, following the incubation, tissue culture medium (such as DMEM/5% FBS with antibiotics) containing 2.5×10⁵ MDCK cells/ml (or other cells) are added to the serum samples (e.g., to all wells of the microtiter plate). In some embodiments, this is incubated overnight in a humidified 37° C., 5% CO₂ incubator. In some embodiments, some influenza viruses grow better at temperatures of 34° to 35° C., and thus those temperatures are used. In some embodiments, the media is removed, and replaced with tissue culture medium (such as DMEM with antibiotics) containing trypsin (such as 0.0002%), and the mixture incubated in a humidified 37° C., 5% CO₂ incubator for 4 days. In some embodiments, subsequently, sterile 0.5% RBC/PBS solution is added, and the mixture incubated at 4° C. for 1 hr, and the wells checked for the presence of agglutination. In some embodiments, the virus neutralization titer of a particular serum sample is defined as the reciprocal of the highest dilution of serum where both wells show no agglutination of the RBC.

In some embodiments, samples (e.g., in a microwell) containing influenza virus neutralizing antibodies at sufficient concentration prevent the virus from infecting the cells so that viral multiplication will not take place. In some embodiments, the addition of RBCs to these wells will result in the formation of a pellet of RBC. In contrast, in some embodiments, samples (e.g., in a microwell) that had none or less than neutralizing concentrations of anti-influenza antibody will have influenza virus present at the end of the 4-day incubation. In some embodiments, the RBC added to these samples will agglutinate. In some embodiments, influenza virus cross-links the red blood cells, inhibiting their settling in the microwell, and the wells therefore appear cloudy.

4. Neuraminidase Inhibiting (NI) Antibody Titer Assay

In some embodiments, neuraminidase inhibiting (NI) antibody titers are determined if a vaccine contains an NA protein. In some embodiments, to measure NI antibody titers, reassortant viruses containing the appropriate NA are generated, for example by using plasmid-based reverse genetics. In some embodiments, the appropriate NA are the same one(s) present in the vaccine administered to the subject. In some embodiments, the NI assay is performed using fetuin as a NA substrate. An exemplary method is provided below.

In some embodiments, the NI titer is the inverse of the greatest dilution of sera that provides at least 50% inhibition of NA activity. In some embodiments, it is expected that use of the VLPs disclosed herein will decrease or even eliminate challenge virus titers in subjects who received the VLPs. In some embodiments, subjects who receive the VLPs are expected to have at least 10-fold, at least 20-fold, at least 50-fold, or even 100-fold less virus in the lungs than subjects who did not receive the VLPs (e.g., are mock vaccinated).

In some embodiments, NI antibody titers are determined in an enzyme-linked lectin assay using peroxidase-labeled peanut agglutinin (PNA-PO) to bind to desialylated fetuin. In some embodiments, NA activity is determined by incubating serial dilutions of purified, full length NA on fetuin coated microtiter plates. In some embodiments, after 30 min incubation at RT, plates are washed, and PNA-PO added. In some embodiments, after 1 h incubation at RT, plates are again washed and the peroxidase substrate 3,3′,5,5′-tetramethylbenzidine added and color development allowed to proceed for 10 min. In some embodiments, color development is stopped and the plates the OD450 measured. In some embodiments, dilution corresponding to 95% NA activity is determined.

In some embodiments, NI titers against an NA subtype are measured beginning at a 1:20 dilution of sera followed by 2-fold serial dilutions in 96-well U-bottomed tissue culture plates. In some embodiments, NAs corresponding to 95% maximum activity are added to diluted sera and incubated for 30 min at RT after which sera/NA samples are transferred to fetuin coated microtiter plates. In some embodiments, plates are incubated for 2 h at 37° C., washed and PNA-PO added. In some embodiments, the plates are incubated at RT an additional hour, washed and peroxidase substrate TMB added. In some embodiments, color development is stopped after 10 min and the OD450 of the plates measured. In some embodiments, the NI titers are the reciprocal dilution at which 50% NA activity is inhibited. In some embodiments, the lower limit of quantitation for the assay is 20; titers lower than 20 are considered to be negative and assigned a value of 10. In some embodiments, a good or positive response produces a value of >30, while a poor or no response produces a value <20.

5. Viral Lung Titers and Pathology

In some embodiments, viral lung titers and pathology are determined. In some embodiments, tissue samples, such as lung samples (e.g., inflated lung samples) are fixed (e.g., 24 h fixation in 10% formaldehyde), embedded (e.g., in paraffin), cut into sections (e.g., 1 to 10 μm, such as 5 μm), and mounted.

In some embodiments, influenza virus antigen distribution is evaluated by immunohistochemistry using an appropriate antibody. In some embodiments, the antibody is a polyclonal or monoclonal antibody that is either specific for the virus used to challenge the subject or one that is cross-reactive to different influenza virus strains. In some embodiments, it is expected that use of the vaccines disclosed herein will decrease or even eliminate virus titers in subjects who received the vaccines. In some embodiments, subjects who receive the vaccines are expected to have at least 10-fold, at least 20-fold, at least 50-fold, or even 100-fold less virus in the lungs than subjects who did not receive the vaccines (e.g., are mock vaccinated). In some embodiments, it is expected that use of the vaccines disclosed herein will decrease or even eliminate symptoms of influenza infection, such as bronchitis, bronchiolitis, alveolitis, and/or pulmonary edema, in subjects who received the vaccines. In some embodiments, subjects who receive the vaccines are expected to have at least 20%, at least 50%, at least 75%, or at least 90% less bronchitis, bronchiolitis, alveolitis, and/or pulmonary edema (or such reductions in severity of these symptoms) as compared subjects who did not receive the vaccines (e.g., are mock vaccinated). In some embodiments, the VLPs are polyvalent.

6. Other Exemplary Assays

In some embodiments, subjects are assessed for respiratory IgA and/or systemic IgG, T-cell responses. In some embodiments, immune responses are analyzed by transcriptomics and cytokine ELISAs or other cytokine immunoassays. In some embodiments, immune responses are analyzed by microneutralization. In some embodiments, immune responses are analyzed by anti-HA stalk assays.

E. Methods of Evaluating a Vaccine

Disclosed herein, in certain embodiments, are methods for determining an effectiveness of a vaccine. Some embodiments include obtaining a sample obtained from a subject who has been administered a vaccine, the sample comprising a presence or an amount of a virus. Some embodiments include providing a substrate comprising an ACE2 or fragment thereof capable of binding to a virus protein. Some embodiments include contacting the substrate with the sample to bind virus or protein virus in the sample to the ACE2 or fragment thereof. Some embodiments include detecting virus or protein virus bound to the ACE2 or fragment thereof of the substrate. Some embodiments include determining the presence or amount of the virus in the sample based on the detected virus or protein virus bound to the ACE2 or fragment thereof of the substrate, thereby determining the effectiveness of the vaccine. In some embodiments, the sample is from a subject. In some embodiments, the sample comprises blood, serum, or plasma. In some embodiments, the virus is a coronavirus. In some embodiments, the virus is a SARS-CoV-2. In some embodiments, the virus protein is a SARS-CoV-2 spike protein. In some embodiments, the amount of virus in the sample is decreased compared to another sample obtained from the subject before the subject was administered the vaccine. In some embodiments, the amount of virus in the sample is increased compared to another sample obtained from the subject before the subject was administered the vaccine. Some embodiments further comprise recommending or providing a virus treatment to the subject based on the amount of the virus in the sample or the effectiveness of the vaccine. In some embodiments, the virus treatment comprises a coronavirus treatment such as a COVID-19 treatment. In some embodiments, the vaccine is a vaccine described herein, such as a vaccine comprising a VLP.

Disclosed herein, in certain embodiments, are methods for determining an effectiveness of a vaccine, comprising: obtaining a sample obtained from a subject who has been administered a vaccine, the sample comprising a presence or an amount of anti-virus antibodies. Some embodiments include providing a substrate comprising a virus protein or fragment thereof capable of binding to the anti-virus antibodies. Some embodiments include contacting the substrate with the sample to bind anti-virus antibodies in the sample to the virus protein or fragment thereof. Some embodiments include detecting anti-virus antibodies bound to the virus protein or fragment thereof of the substrate. Some embodiments include determining the presence or amount of the anti-virus antibodies in the sample based on the detected anti-virus antibodies bound to the virus protein or fragment thereof of the substrate, thereby determining the effectiveness of the vaccine. In some embodiments, the sample is from a subject. In some embodiments, the sample comprises blood, serum, or plasma. In some embodiments, the virus is a coronavirus. In some embodiments, the virus is a SARS-CoV-2. In some embodiments, the virus protein is a SARS-CoV-2 spike protein. In some embodiments, the amount of anti-virus antibodies in the sample is decreased compared to another sample obtained from the subject before the subject was administered the vaccine. In some embodiments, the amount of anti-virus antibodies in the sample is increased compared to another sample obtained from the subject before the subject was administered the vaccine. Some embodiments further comprise recommending or providing a virus treatment to the subject based on the amount of the anti-virus antibodies in the sample or the effectiveness of the vaccine. In some embodiments, the virus treatment comprises a coronavirus treatment such as a COVID-19 treatment. In some embodiments, the vaccine is a vaccine described herein, such as a vaccine comprising a VLP.

### IX. EXAMPLES

The following examples are included for illustrative purposes only and are not intended to limit the scope of the invention.

### Example 1

### Use of VLP Vaccines

After the selection of optimal broadly cross-reactive VLP (e.g. seVLP or smVLP) vaccines in experimental animals, studies will be conducted in human with polyvalent influenza seVLPs (for example that are produced using the Good Manufacturing Practice (GMP) such as from Paragon Bioservice, Baltimore, Md.). In some embodiments, the VLPs also contain M1 and M2. The polyvalent VLP, in some embodiments, also contains MPL as the adjuvant.

A polyvalent vaccine formulation that comprises of mixture of HA VLPs separately presenting H1, H2, H3, H5, H7, and H9, and NA VLPs separately presenting N1 and N2 will be generated using GMP methods and administered to humans by microinjection. In some embodiments, other polyvalent influenza vaccines that are not described herein are tested.

Briefly, humans are vaccinated by microneedle injection with a VaxiPatch microneedle array comprising trehalose sugar glasses with a polyvalent mixture of VLPs (10 μg-20 μg, such as 15 μg each HA/NA). About 3-12 weeks later (such as 3, 4, 5, 6, 7, 8, 9, 10, 11, or 12 weeks later), the humans are boosted with the same mixture. A second group of humans are mock vaccinated (for example with saline). In some embodiments, blood samples are obtained and stored. Patients will be monitored for any adverse events (AEs) during the course of study. Since VLP vaccines are not infectious, they are expected to have an excellent safety profile.

The VLP is shown to be safe in Phase I trials, and Phase II efficacy trials are performed using a human influenza challenge model, as developed at the NIH Clinical Center (e.g., see Memoli et al., Validation of a Wild-Type Influenza A Human Challenge Model: H1N1pdMIST, An A(H1N1)pdm09 Dose Finding IND Study). Subjects are screened for health status and by HA1 assay for low titers (<1:10) against the challenge 2009 pandemic H1N1 virus. Screened patients enrolled in the study are vaccinated by microneedle injection as described with the polyvalent mixture of VLPs (cohort 1) or given a mock vaccination with saline (cohort 2). They are boosted at three weeks, and then at six weeks their serologic titers are assessed by HA1 or other assays, and the subjects are challenged with a dose of virus validated to induce influenza illness and shedding in >60% subjects pre-challenge HA1 titers <1:10. Vaccine efficacy are assessed by development of serologic responses to vaccination, reduction in symptoms, reduction in viral titers, and/or reduction in duration of viral shedding.

### Example 2

### Vaccination Against Influenza

Rats vaccinated by microneedle injection (to induce systemic immunity) with monovalent HA seVLPs or with monovalent HA smVLPs are protected from heterologous lethal influenza challenge. Additionally, rats that are vaccinated with a TLR agonist as an adjuvant exhibit reduced morbidity compared to those that receive a similar vaccine not comprising an adjuvant. In some cases, polyvalent seVLP or smVLP mixtures protect against lethal influenza A viruses such as 1918 H1N1, 1957 H2, 2004 H5N1, and 2013 H7N9.

### Example 3

### Non-Limiting Exemplary Methods

Cloning, expression, and protein purification: The gene sequence of an antigen is synthesized and cloned in the expression vector pET-28a (+)between Ndel and BamH1 restriction sites. Cloning is confirmed by sequencing. Constructs are codon-optimized for expression in E. coli.

Proteins are over-expressed in E. coli BL21 (DE3) cells and purified from the soluble fraction of the cell culture lysate. A single colony of E. coli BL21(DE3) transformed with a plasmid comprising a nucleic acid encoding an antigen of interest is inoculated into 50 ml of Tartoff-Hobbs HiVeg™ media (HiMedia). The primary culture is grown over-night at 37 degrees C. 2 L of Tartoff-Hobbs HiVeg media (500 ml×4) (HiMedia) is inoculated with 1% of the primary inoculum and grown at 37 degrees C. until an OD₆₀₀ of ˜0.6-0.8 is reached. Cells are then induced with 1 mM isopropyl-beta-thiogalactopyranoside (IPTG) and grown for another 12-16 hours at 20° C. Cells are harvested at 5000 g and resuspended in 100 ml of phosphate-buffered saline (PBS, pH 7.4). The cell suspension is lysed by sonication on ice and subsequently centrifuged at 14,000 g. The supernatant is incubated with buffer-equilibrated Ni-NTA resin (GE HealthCare) for 2 hours at 4° C. under mild-mixing conditions to facilitate binding. The protein is eluted using an imidazole gradient (in PBS, pH 7.4) under gravity flow. Fractions containing the protein or antigen of interest are pooled and dialysed against PBS (pH 7.4) containing 1 mM EDTA. The dialysed protein is concentrated in an Amicon (Millipore) stirred cell apparatus to a final concentration of about 1 mg/ml. Protein purity is assessed by SDS-PAGE and its identity confirmed by ESI-MS. In some embodiments, the polypeptides or antigens are produced in other expression systems besides E. coli such as yeast, plant, and animal using expression system specific promoters or codon optimized DNA sequences that encode the polypeptides or antigens.

Immunization and challenge studies: Female Sprague Dawley rats (4-5 weeks old) are used. Rats (10/group) are immunized intramuscularly with 20 μg of test immunogen along with 100 μg CpG7909 adjuvant (TriLink BioTechnologies, San Diego, Calif) at days 0 (prime), and/or 28 (boost). Naive (buffer only) rats and/or adjuvant-treated rats are used as controls. Serum sample obtained from tail vein venipuncture are collected in Microtainer serum separator tubes (BD Biosciences, Franklin Lakes, N.J.) 21 days after the prime and/or 14 days post boost from the rats. 21 days after the primary and/or secondary immunization, rats are anesthetized with ketamine/xylazine and challenged intranasally with ILD₉₀ of rat-adapted virus in 20 μL of PBS. In order to test for protection against a higher dose of the virus, one group of rats primed and boosted with an antigen is challenged with 2LD₉₀ of homologous virus. The ability of the vaccine to confer protection is evaluated. Survival and weight change of the challenged rats are monitored daily for 14 days post challenge. At each time point, surviving rats of a group are weighed together and the mean weight calculated. Errors in the mean weight are estimated from three repeated measurements of the mean weight of the same number of healthy rats.

Determination of serum antibody titers: Antibody-titers against test immunogens are determined by ELISA. Test immunogens are coated on 96-well plates (Thermo Fisher Scientific, Rochester, N.Y.) at 4 μg/ml in 50 μl PBS at 4° C. overnight. Plates are then washed with PBS containing 0.05%Tween-20 (PBST) and blocked with 3% skim milk in PBST for 1 h. 100 μl of the antisera raised against the test immunogens is diluted in a 4-fold series in milk-PBST and added to each well. Plates are incubated for 2 h at room temperature followed by washes with PBST. 50 μl of HRP-conjugated goat anti-mouse IgG (H+L) secondary antibody in milk-PBST is added to each well at a predetermined dilution (1:5000) and incubated at room temperature for 1 h. Plates are washed with PBST followed by development with 100 μl per well of the substrate 3, 3′,5,5′-tetramethylbenzidine (TMB) solution and stopped after 3-5 min of development with 100 μl per well of the stop solution for TMB. OD at 450 nm is measured and the antibody titer is defined as the reciprocal of the highest dilution that gave an OD value above the mean plus 2 standard deviations of control wells.

### Example 4

### B/Colorado/06/2017 rHA Construct Design, Expression and Purification

B/Colorado/06/2017 (B/CO′17) recombinant HA (rHA) was designed with a thrombin cleavage site leading to a 6×HIS tag at the C-terminus of the HA. Once cleaved, the B/CO′17 protein product would only include three residual amino acids (Val-Pro-Arg) appended to the wild-type sequence. The amino acid sequence of the synthetic construct was as follows:

(SEQ ID NO: 15)
MKAIIVLLMVVTSSADRICTGITSSNSPHVVKTATQGEVNVTGVIPLTTT
PTKSHFANLKGTETRGKLCPKCLNCTDLDVALGRPKCTGKIPSARVSILH
EVRPVTSGCFPIMHDRTKIRQLPNLLRGYEHVRLSTHNVINAEGAPGGPY
KIGTSGSCPNITNGNGFFATMAWAVPDKNKTATNPLTIEVPYVCTEGEDQ
ITVWGFHSDNETQMAKLYGDSKPQKFTSSANGVTTHYVSQIGGFPNQTED
GGLPQSGRIVVDYMVQKSGKTGTITYQRGILLPQKVWCASGRSKVIKGSL
PLIGEADCLHEKYGGLNKSKPYYTGEHAKAIGNCPIWVKTPLKLANGTKY
RPPAKLLKERGFFGAIAGFLEGGWEGMIAGWHGYTSHGAHGVAVAADLKS
TQEAINKITKNLNSLSELEVKNLQRLSGAMDELHNEILELDEKVDDLRAD
TISSQIELAVLLSNEGIINSEDEHLLALERKLKKMLGPSAVEIGNGCFET
KHKCNQTCLDKIAAGTFDAGEFSLPTFDSLNITAASLNDDGLDNHTILLY
YSTAASSLAVTLMIAIFVVYMVSRDNVSCSICLVPRGSHHHHHH.

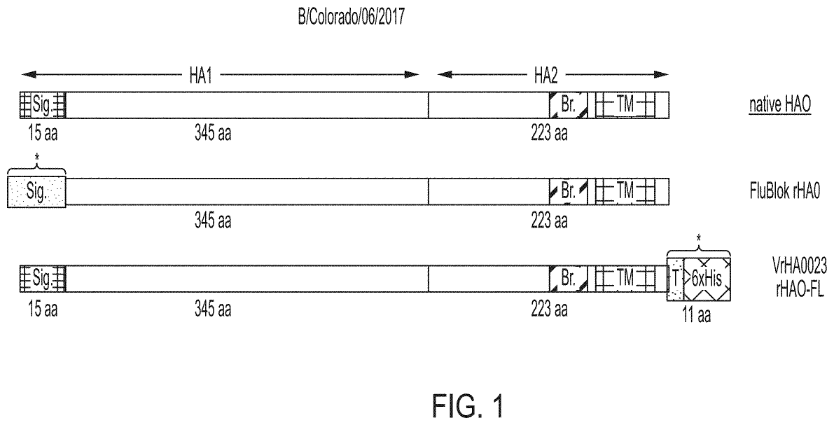

The underlined sequence represents the synthetic thrombin cleavage site, while the last six amino acids are the C-terminal 6×His tag. A drawing showing the native influenza HAO, the HAO of FluBlok® from Sanofi and the Verndari rHA00023 construct is shown in FIG. 1.

ATUM bio was used as a synthesis vendor. The pD2600-v10 plasmid backbone was used. This vector was designed for high-level transient expression and bears a Kanamycin resistance gene for bacterial selection. After sequence optimization for CHO cells, the DNA sequence was in accordance with SEQ ID NO: 16.

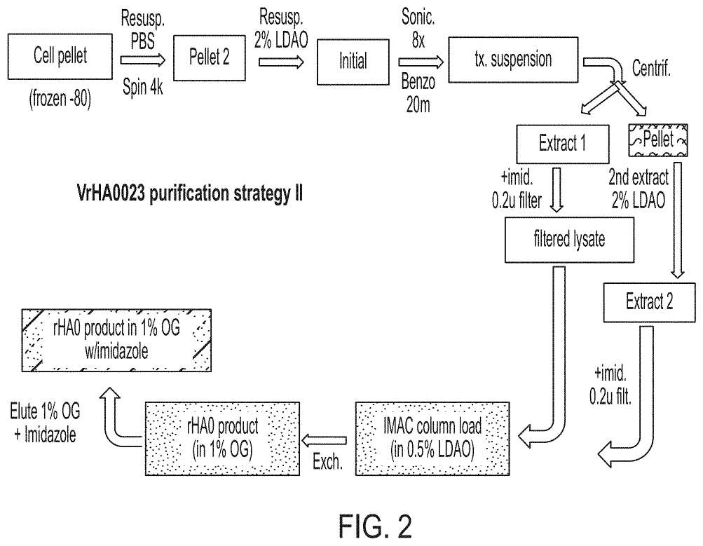

ExpiCHO-S cells (Fisher) were expanded at passage P4 to two E250 flasks, from a vial frozen at P1. This expansion culture attained a density of 8.556×106. Five E125 flasks were prepared with 150M cells each in 25 mL of media. One E250 flask was also prepared with 300M cells in 50 mL of media. Transfections were performed using 12.33 uL of plasmid stocks at 1 ug/mL. At 19 hours post-transfection, enhancer and feed reagents were added to transfection cultures, and initial density and viability evaluations made by trypan blue exclusion. These evaluations were thereafter performed daily using 0.4 mL of suspension culture. the transfected cell pellets retain the vast majority of the recombinant HA. The flow chart for purification of B/Colorado/06/2017 rHAO is shown in FIG. 2.

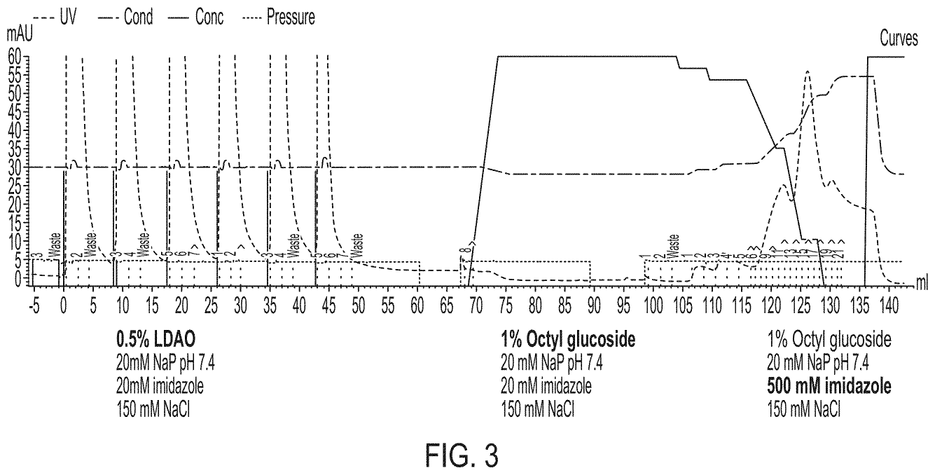

A lysis buffer that was used was made up of 20 mM phosphate buffer (pH 7.4), 150 mM NaCl, and 2 mM MgC12 (to support Benzonase activity) and 2% LDAO detergent (n-Dodecyl-N,N-Dimethylamine-N-Oxide, Anatrace). The LDAO detergent was exchanged to 1% octyl glucoside detergent on the IMAC column. FIG. 3 shows the loading of lysate in the IMAC column, the detergent exchange and the elution of rHA.

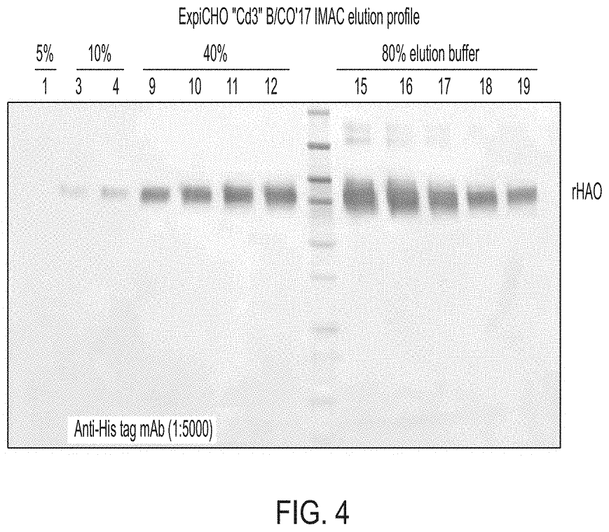

The western blot in FIG. 4 shows the rHA elution profile with the gradient of 500 mM imadozole. Pooled rHA was concentrated and buffer exchanged to PBS containing 1%. This rHA was used to produce synthetic membrane VLPs.

### Example 5

### Liposome Production

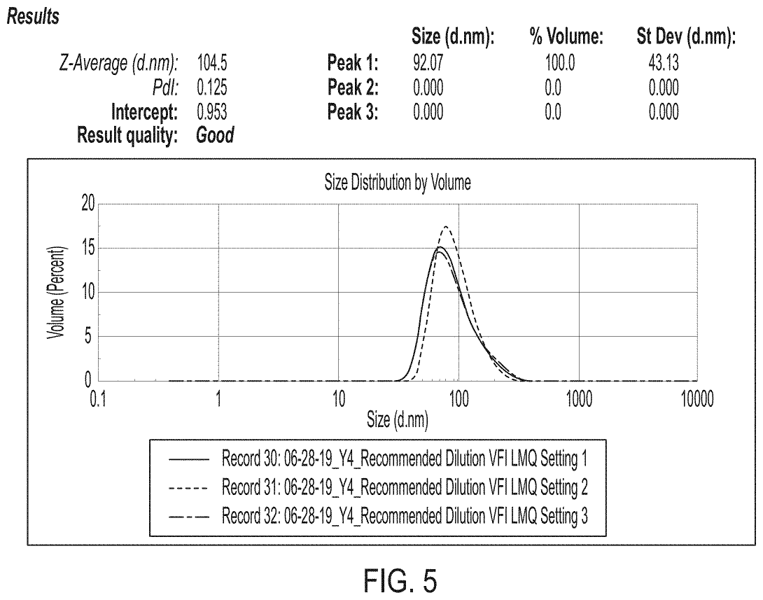

Imiquimod (IMQ) was formulated into liposomes. Liposomes were formed using a NanoAssemblr, Precision NanoSystems, Vancouver, BC. The aqueous phase was PBS. The organic phase consisted of 25 mg/mL lipid mix and 3.5 mg/mL IMQ in ethanol. The flow rate was 8 ml/minute. The flow rate ration of aqueous to organic was 2.5. Liposomes were immediately diluted 10-fold with PBS. Ethanol and unincorporated IMQ was removed with a 30 Kd Amicon filtration column and 4000 g centrifugation. The Amicon retentate was diluted with PBS and the Amicon filtration was repeated. Lipsomes were sized by dynamic light scattering (DLS) using a Malvern Zetasizer-NS. As shown in FIG. 5, the size of the liposomes averaged 92 nm.

The IMQ was quantitated in the liposomes by HPLC. The HPLC contained a Waters Alliance instrument with an Xterra C18 Column (MS C18 5 um 4.6×150 mm Column), 2998 Photodiode array detector, 2525 binary gradient module, and UV fraction manager. The mobile phase was 15% acetonitrile and 0.1% trifluoroacetic acid. This system gave a linear dose response curve of IMQ from 60 uM-3 mM IMQ.

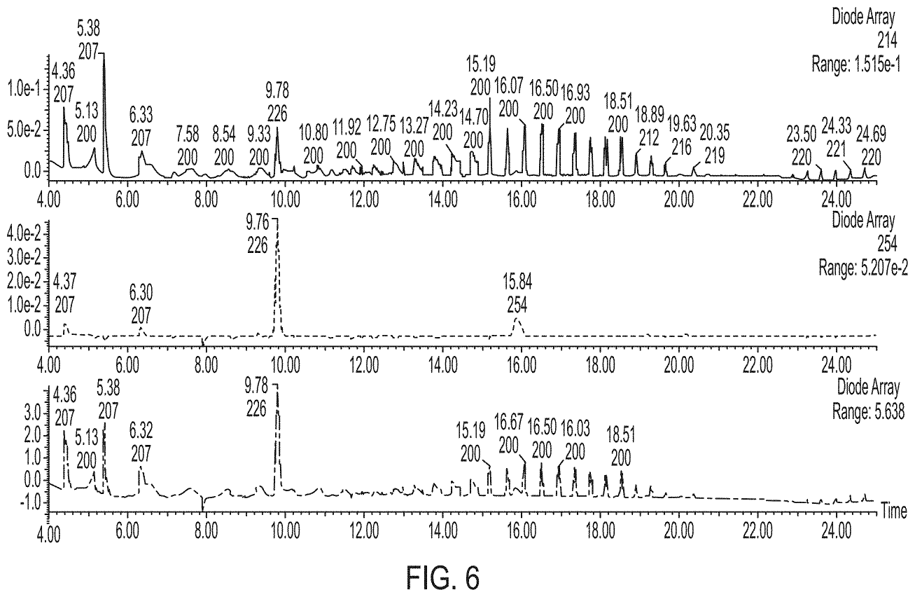

FIG. 6 shows UV scans at 242 nm, 245 nm and 254 nm of IMQ containing liposomes. IMQ is eluting at 9.78 minutes. These liposomes containing IMQ were used as adjuvant formulated in 15% trehalose with seVLPs printed on VaxiPatch microarrays and used in the animal experiments.

### Example 6

### Production of seVLPs

The rHA of B/Colorado/06/2017 of Example 4 was used in two ways to make seVLPs. The first way of making seVLPs included dialsysis: For reconstitution as seVLPs, 2 mg of lipids (phosphatidyl choline (50 mg/ml), cholesterol (20 mg/ml), phosphatidyl ethanolamine (10 mg/ml), phosphatidyl serine (10 mg/ml), sphingomyelin (20 mg/ml) and phosphatidyl inositol (2.5 mg/ml) mixed in a ratio of 10:4.25:3:1:3 and 0.5% respectively) were dissolved in 400 μl 10% OG. 500 ug of B/Colorado/06/2017 rHA was then added to the dissolved lipids and the total volume was made up to 2 ml, giving an end concentration of 4% OG. The 2 ml sample was dialysed against numerous changes of small volumes (3 ml) of PBS for 24 hours at 4 ° C. The sample was then dialyzed against 4×12 ml PBS over 24 hours. The sample was then transferred to 2×2.5 L for 24 hours, and finally transferred to 5 L of PBS for 48 hours to remove OG. seVLPs were 100-200 nM in size as determined by dynamic light scattering (DLS) using a Malvern Zetasizer-NS.

The second way of making seVLPs included a NanoAssemblr. Based on the critical micelle concentration (c.m.c.) of OG at 25 mM, seVLPs were formed by reducing the OG from 30 nM to 20 mM while mixing with lipids. Influenza rHA protein in aqueous buffered saline and 30 mM OG was mixed with DOPE, DOPC, cholesterol and DSPE-PEG(2000) Amine in ethanol. The aqueous to organic volume ratio was 2:1. The flow rate was 8 ml/minute. seVLPS were collected into PBS and buffer exchanged to PBS and concentrated using Amicon 30 kd columns. seVLPs were 100-200 nM in size as determined by dynamic light scattering (DLS) using a Malvern Zetasizer-NS.

The activity and potency of rHA B/Colorado/06/2017 in the seVLPs was determined by hemagglutination and SRID.

### Example 7

### BioDot Printing of Vaccine on VaxiPatch Microarrays

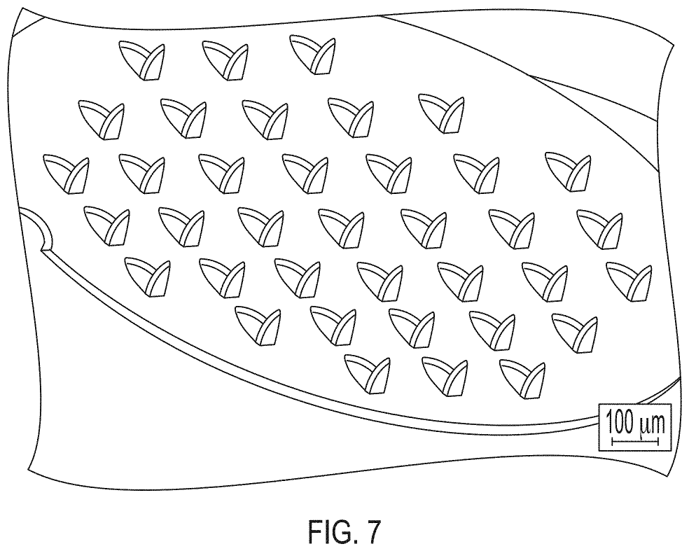

VaxiPatch MicroArray Patches (MAPs) were designed to utilize BioDot (Irvine, Calif.) microfluidic dispensing devices. This dispensing was done in two dimensions (X, Y). The individual VaxiPatch MAPs were circular, 1.2 cm in diameter, each with 37 individual MicroTips. The MAPs were loaded with vaccine in trehalose using a BioDot microfluidic dispenser. In manual mode, all 37 individual microtips were loaded in the two-dimensional X, Y plane with 5 to 20 nL per tip in 10 seconds. Scaling up of a custom-designed dispensing device allows parallel dispensing of 10 arrays at a time, yielding a throughput of several hundred arrays per minute. Once the MicroArrays were loaded and dried, the arrays were placed in a sandwich jig. The jig contained pegs that corresponded to the array such that when the sandwich was compressed, the MicroTips were bent into the Z plane. The individual MAPs were then punched out with a die. Room stability was achieved with the presence of a desiccant. lug rHA and lug adjuvant was formulated in 15% trehalose in PBS. The mixture was then printed onto the VaxiPatch microarrays using a BioDot AD1520. Upon drying and vitrification sugar glasses were formed. FIG. 7 shows a single microneedle of a VaxiPatch microneedle array loaded with 10 nL of vaccine containing a blue dye No. 1. The light reflection in the figure shows the surface of the solid sugar glass. The potency of rHA B/Colorado/06/2017 was shown by SRID.

### Example 8

### Animal Studies

seVLPs presenting the rHA from B/Colorado/06/2017 were pooled and concentrated using Amicon Ultra-0.5 spin diafiltration columns with 30 kD cutoff membranes. The vaccine material (1.62 mL) was centrifuged for 30 min at 13 k RPM in a pre-chilled centrifuge rotor. Retentate was then eluted with a 1-minute spin at 13 k RPM before formulation. Assuming full retention and release of rHA by the columns, the initial concentrated material was estimated at 3.24 mg/mL for the rHA protein. Formulated at 1:1 with 30% trehalose (with or without 4% BB dye), this equated to 0.389 ug of rHA/array when printed with single 10 nL drops. The resulting material was 15% trehalose with or without 2% BB for visualization and delivery assessment. For lower dosage concentrated rHA was estimated to have been 2.32 mg/mL for each rHA protein. This material was then diluted with nuclease-free water and formulated to prepare the 0.2 ug/rHA and 0.04 ug/rHA printing doses in 15% trehalose, with 2% Brilliant Blue FCF dye.

Sprague-Dawley rats, with hair previously removed, were treated with these arrays utilizing 5 minute direct pressure; a method that was demonstrated to be capable of roughly 90% release of vaccine material from the MicroArray patches. The application site selected was the midline of the back, and animals were treated while under isoflurane. All animals were maintained with weekly blood draws for assay of immune responses to the seVLP B/Colorado/06/2017 vaccine.

Another control group of three animals received intradermal injections of 0.2 ug/seVLP B/Colorado/06/2017 (diluted in sterile phosphate-buffered saline (PBS). Efficiency of treatment delivery was estimated to be over 90% for all dye-formulated MicroArray Patch treatments based on comparisons dye elutions from parallel-printed, non-applied arrays with retained dye on post-treatment arrays.

Weekly blood draws were conducted through week 4, at which point the animals were humanely euthanized and a terminal draw collected by cardiac puncture. Serum from these “week 4” bleeds was analyzed for reactivity to B/Colorado/06/2017 rHA by ELISA assay.

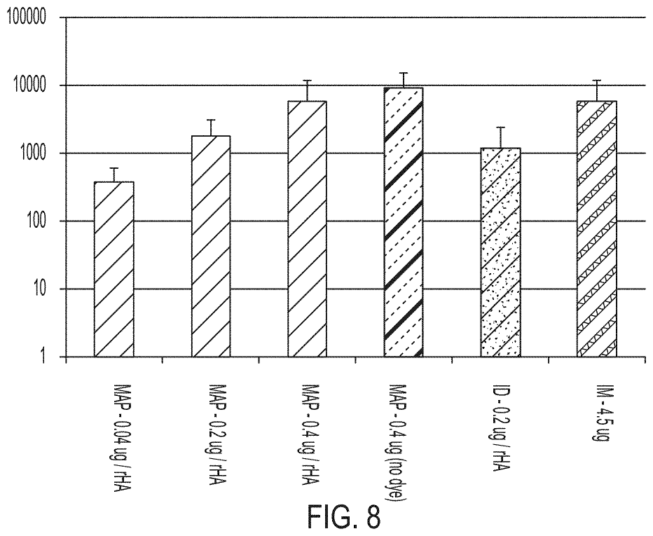

ELISA Assay: Plates were coated overnight at 4° C. with rHA protein (B/Colorado/06/2017) at 0.5 μg/ml in 100 mM Carbonate buffer. The plates were then washed 3x with Tween-20 (TBST) and blocked with 5% BSA in TBS for 1 h at room temperature. After washing, rat sera (1:100-1:12500) and positive control antibody (1:62,500-1:7,812,500; monoclonal anti-HA-antibody, ImmuneTech in 1% BSA/TBST were added and incubated for 2 hours at room temperature, followed by washing. Goat anti-rat-HRP antibody (Jackson Labs, 112-035-143), at 1:20,000 was used. Data are shown in FIG. 8. In FIG. 8, MAP=microarray skin patch; IM=intramuscular injection; the Y-axis is the dilution of serum used in the ELISA test.

### Example 9

### VaxiPatch Kit for Human Vaccination

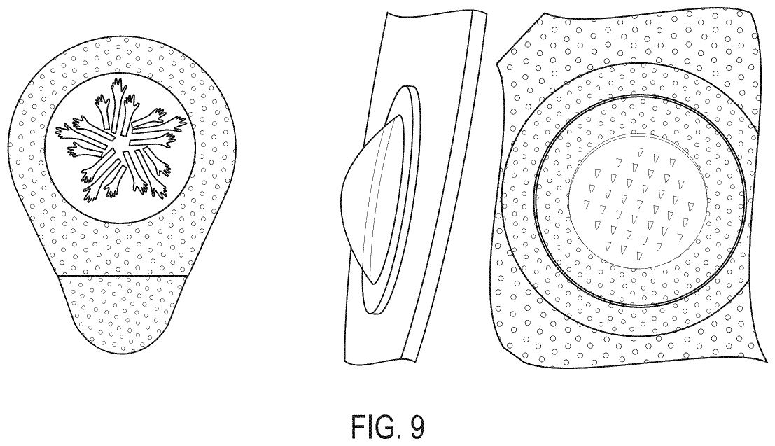

An example of a VaxiPatch is shown in FIG. 9, which includes images of the back (left panel), side (middle panel), and back (right panel) of the VaxiPatch. The front side is placed on the skin of a subject upon administration. The right panel shows a vaccine-loaded 1.2 cm diameter MAP.

Vaccine administration: The layers of the VaxiPatch device are pulled apart, removing the clear dome covering the MAP; the MAP is placed on the skin approximately 1″ proximal to the ulnar knob of the wrist. The center of the Verndari logo (shown in the left panel of FIG. 9) is pressed with the index finger with approximately six Newton's of force when the device emits an audible click, which indicates enough force has been exerted. The MAP is propelled into the skin in a highly reproducible manner. The device remains on the skin for 10 minutes held in place by 3M medical adhesive. After 10 minutes the VaxiPatch is removed, placed back in the pouch, sealed with a zip lock seal, and discarded as medical waste.

The moisture in the skin dissolves the vaccine off the MAP, the vaccine enters the skin and is processed by professional antigen presenting cells such as dendritic and Langerhan cells. The vaccination is painless as the microneedles are 600 μm in length and too short to reach a nerve.

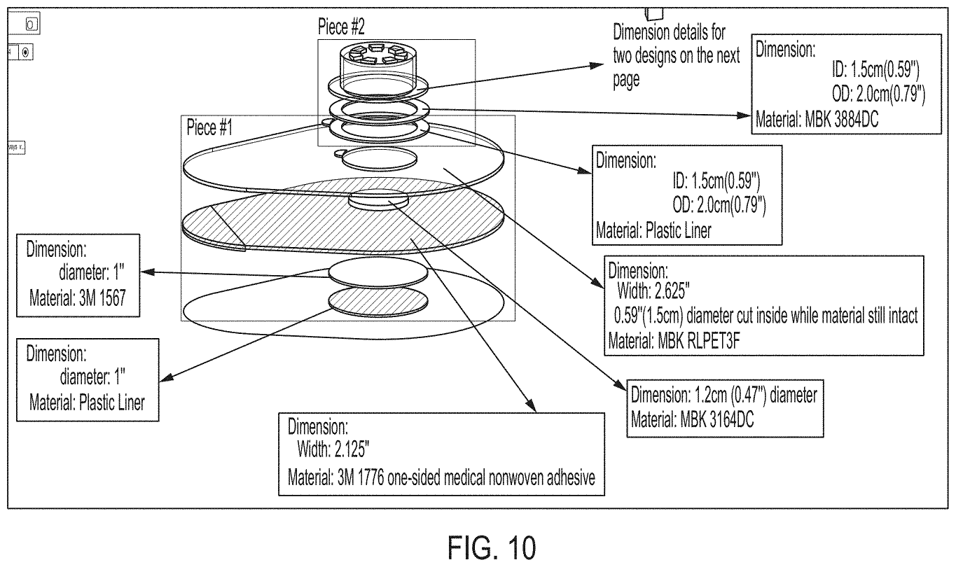

The clear plastic dome shown in FIG. 9 (middle and right panels) provides a primary sterility barrier for the vaccine on the MAP and protects the microneedles. However, the dome is not gas tight. The VaxiPatch device is packaged in a secondary gas tight barrier envelope along with a skin wipe towelette and desiccant. The desiccant and gas tight barrier envelope maintain a dry environment that aids in maintaining the integrity of the vaccine sugar glass providing room temperature stability. FIG. 10 shows a schematic drawing showing an expanded view of an example of a VaxiPatch.

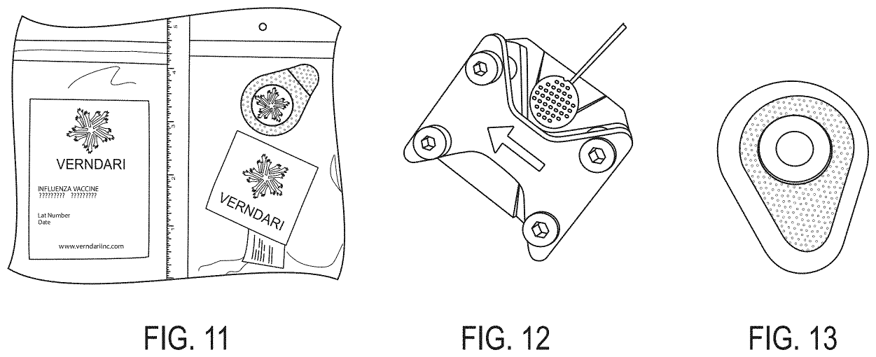

An example of a VaxiPatch vaccination kit is shown in FIG. 11. Shown is a two sided re-sealable 4″×7″ pouch containing a VaxiPatch, a skin wipe and a desiccant. The kit does not include a traditional needle or syringe. The pouch is gas tight with a foil front and clear plastic back. The pouch is ¼″ in width at its thickest point.

### Example 10

### VaxiPatch Assembly

A purpose of the procedure described in this example is to demonstrate ways to prepare, formulate, and print a vaccine to designated half-etched wells of a microarray patch.

The procedure described in this example is designed to effectively assemble and package the prepared VaxiPatch into the individual pouch with the desiccant prior to the final drying and storage.

Examples of equipment and materials to be used in some embodiments in assembling a VaxiPatch include, but are not limited to, the following:
Sterile drying Tray
Stainless Steel forceps, type PL-30 (Fisherbrand 12-000-122)
Microscope (Celestron, Model #: OMAX 40X-2500X)
Custom Stainless Steel Array Printing tray (Verndari Inc)
Custom Stainless Steel Bending jig (Verndari Inc)
Custom Stainless Steel Snap Applicator (Verndari Inc, manufactured by Weichhart Stamping Co.)
Individually packed 3g desiccant bag (DrieRite®, Cat #: 60013T)
GMP-grade Heat-Sealer (Accu-Seal, Model #: 8000-GV)
Dry Argon compressed gas cylinder (Harris gas)
Foilpak Pouch 5″×8″-4.5 mL: Foil & Polypropylene Three-Side-Seal Barrier Pouch (AMPAC Flexibles, item #: KSP-150-1MB)
Pre-assembled packaging piece #1 (internally designed and manufactured by 3M MBK Tape)
Double sided ring-shaped tape (internally designed and manufactured by 3M MBK Tape)
Sterile clear dome (internally designed and manufactured by UC Davis TEAM Lab)
FoilPak: Thin Metal Pouch

Non-limiting, exemplary instructions are as follows:

For packaging, prepare a sterile printed array, custom stainless-steel bending jig, stainless steel forceps, pre-assembled packaging piece #1, and double-sided ring-shaped tape. Gather the following items on a sterile flat surface: double-sided ring tape; clear dome; snap applicator; pre-assembled packaging support material; forceps; printed array; bending jig.

Insert the printed array to the bending jig as shown in FIG. 12. The arrow in FIG. 12 indicates the direction where the top of the microarray tips is to be located. Insert the array facing the printed well-side down, with the tips pointing up to match the arrow.

Next, press the bending jig firmly to tilt the microarrays to initiate the microneedles in a proper skin-applicable form. For example, after pressing down the array, and taking it out, the array will comprise microneedles extending 90 degrees from the metal plane from which the microneedles extend. Set aside the bent array on the sterile surface using the sterile forceps.

Next, a pre-assembled packaging set including a support material and a metal snap applicator is obtained. In some embodiments, the metal snap applicator is in a “pre-actuating” form, which is the ready-to-apply form.

Flip the support material to show the white circular backing is facing up. Carefully remove the opaque white circular tape backing piece to expose the circular tape.

Next, align the circular tape and the metal snap applicator (convex form facing up) and carefully press the edge of the metal snap applicator to be firmly attached to the support material as shown in FIG. 13. Next, flip the assembled piece to have the other side facing up.

Remove the top-most clear tape backing to expose the small circular adhesive. Carefully align the prepared 90 degree-bent array (microneedle facing up), and attach to the support material. Gently tap the outer edge of the array using the sterile metal forceps.

Next, obtain the circular ring tape and remove the ring-shaped white tape backing using the gloved hand. Obtain the clear dome and properly align and attach the clear dome to the circular tape. Gently tap the edge of the clear dome to make sure the firm attachment.

Next, remove the larger backing to separate the clear dome+double-sided ring adhesive and carefully place it on top of the array. Note that the proper alignment is essential for this step since the clear 3D dome is supposed to have a function of protecting the microneedle-bent-array.

Next, obtain the completed assembled VaxiPatch piece, a 3 g desiccant package, and a thin metal pouch (e.g. Foilpak). Place the 3 g desiccant package first and carefully place the assembled VaxiPatch Piece into the metal pouch.

Turn on the heat sealer. Turn on the argon gas valve in order to provide the accurate pressure for the heat sealer. Select the “Recipe 1”. Hold the open interface of the pouch fin between the two sealing gaskets. When the pouch is properly positioned, and fingers are safely removed from the sealing area, use the rocker pedal to initiate the thermal sealing. An audible beep will sound to indicate completion, and the sealing surfaces will separate. The sealed pouch may then be removed. Light pressure may be required to separate it from the lower sealing gasket. Seal and store the pouch at 20° C.

### Example 11

### Point-of-Care Vaccines

A purpose of the procedure described in this example is to demonstrate ways to provide point-of-care vaccines for infections causing illnesses such as Influenza, Rabies, Shingles, COVID19, and so forth. Some examples include a vaccination kit, are room-temperature stable (e.g., for mail distribution), can be self-aadministration by, for example, a painless five-minute bandage, allow for photo proof of vaccination (e.g., via a mobile device), can be mail to vendors in, for example, a plastic storage bag.

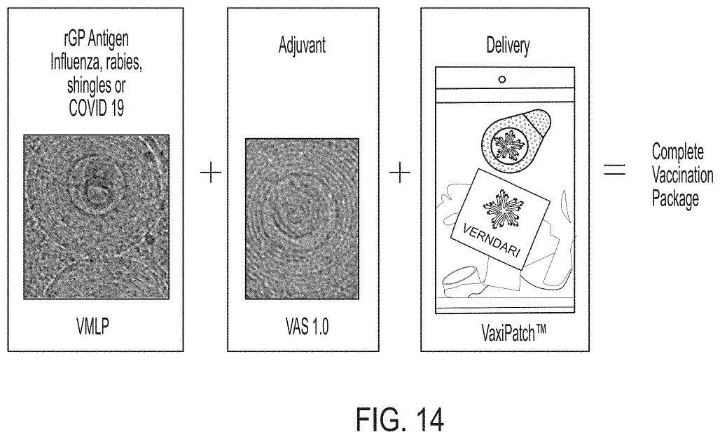

FIG. 14 shows an example three-pronged approach to address the point-of-care vaccination problem. The example shows how an rGP Antigen, an adjuvant, and delivery are brought together to provide a complete vaccination package. In some embodiments, the rGP is a recombinant glycoprotein from the surface of a virus.

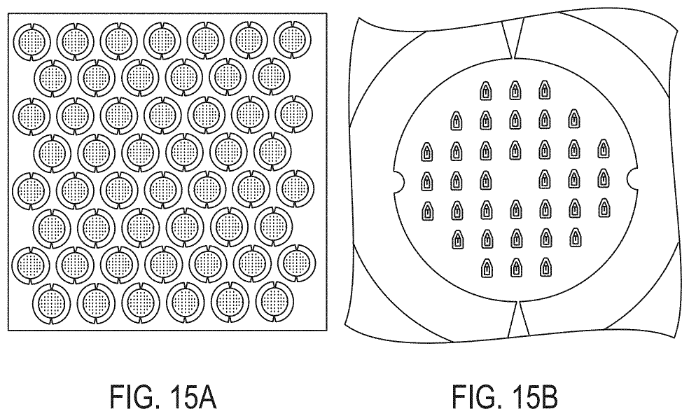

FIGS. 15A and 15B show example sheets of microneedle arrays. In some embodiments, these sheets comprise medical grade stainless steel. In some embodiments, the microneedle arrays print vaccine in two dimensions (X, Y). In some embodiments, a jig can be employed to tilt the microneedle in the array in the Z-plane. In some embodiments, a central spot vacuum pick can be employed to spread and place to enable automated assembly of a VaxiPatch kit.

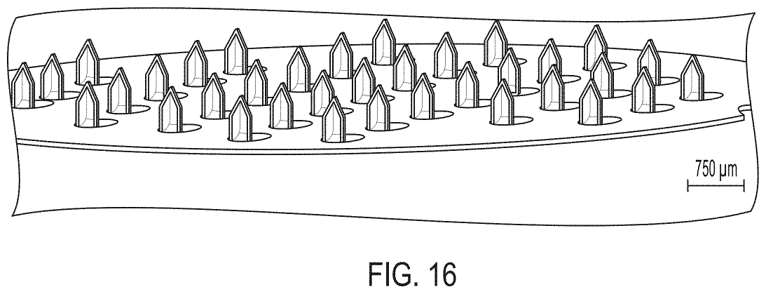

FIG. 16 shows an example of a vaccine loaded microarray. The depicted example shows BioDot printing of 10 nL vaccine print mix/microneedle.

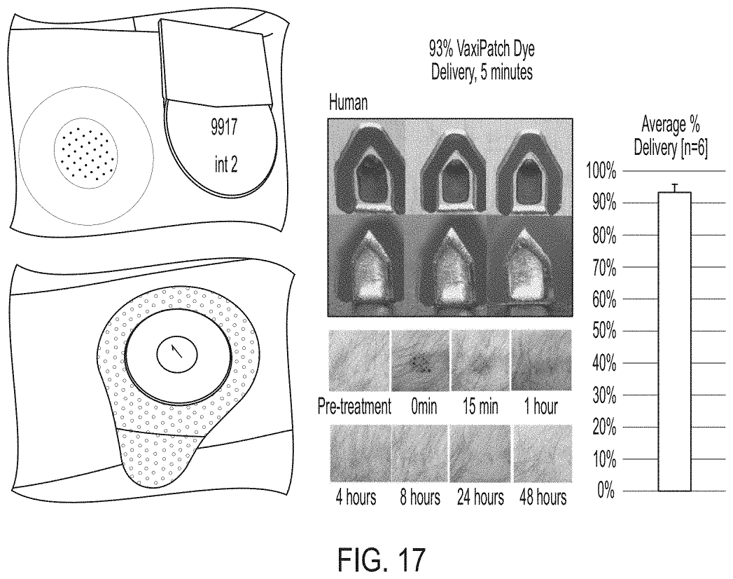

FIG. 17 shows an example of a VaxiPatch dye delivery in five minutes in a human subject.

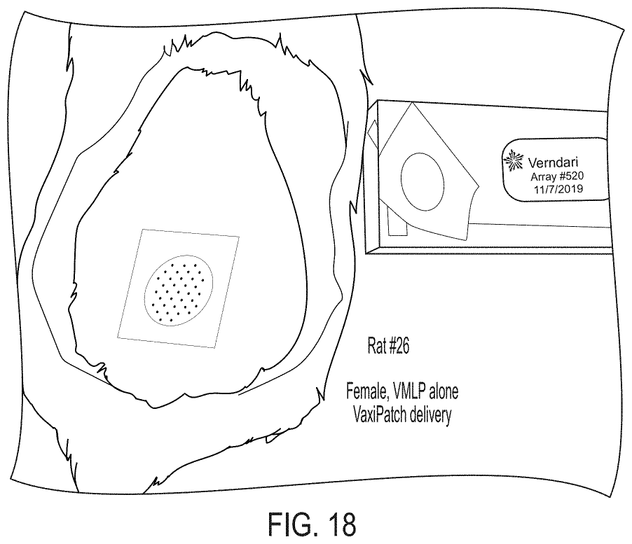

FIG. 18 shows an example of a VaxiPatch dye delivery in a rat. The example included a dose of 0.3 ug of monovalent rHA as MLPVi, 0.5 ug of QS-21+/−(0.3 ug PHAD) as VAS 1.0, 0.5% FD&C with no.1 blue dye (w/v), 1/150th rHA of Flublok, and 1/100th QS-21 as Shingrix. The example VaxiPatch arrays were applied for 5 minutes with n=6 per group (3 males, 3 females), Sprague-Dawley rats, Pre-immune and weekly bleeds, followed by a 28-day terminal bleed. The Draize assessments for skin redness/irritation showed no irritation from VaxiPatch, dose or formulation.

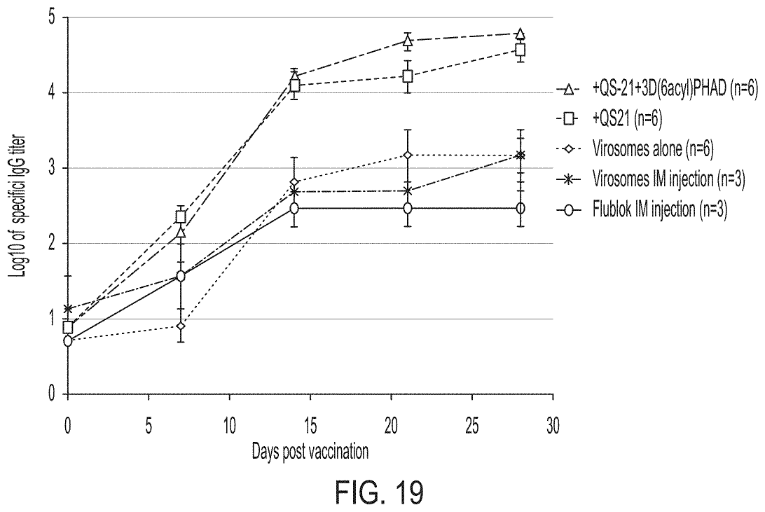

FIG. 19 shows VaxiPatch Rat ELISA titers with an IgG timecourse. As depicted, levels of IgG antibody specific for HA from B/Colorado/06/2017 were assessed in the serum of vaccinated Sprague-Dawley rats by ELISA assay against an in-house full-length rHAO protein (VrHA0026). Endpoint titers were assigned based on five-fold dilution series across an N of 6 animals per group (3 males and 3 females each). The titers were log10-transformed, and averages used for plotted data points. Error bars represent SEM for an N of 6 per group. An arbitrary titer of “5” was assigned to samples negative at the initial 1:100 dilution (presumed to be non-responders). Both adjuvated formulations shown exhibited high levels of specific IgG as early as 14 days post-vaccination, with peak levels by week 3-4. Adjvuanted VaxiPatch animals achieved substantially higher endpoint titers than IM injection comparators at all time points beyond day 7.

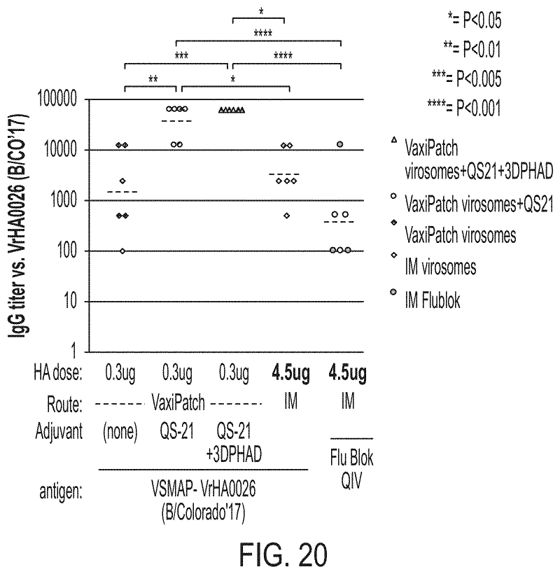

FIG. 20 shows VaxiPatch ELISA titers to B/Colorado 2017. The figure shows the individual variation within each vaccination group at the final day 28 timepoint, with a marker for each animal. Darker shaded markers represent female animals. Geometric means are represented by dashed lines for each group. Intramuscular injection control animals received a single dose of 4.5 micrograms of antigen, while VaxiPatch animals received 0.3 micrograms of protein. Note that the FluBlok dose was selected to include 4.5 micrograms of each strain, as it is a quadrivalent product (18 micrograms total protein). Statistical significance between groups is indicated above the graph, based on a one-way ANOVA and Tukey's HSD post-hoc test.

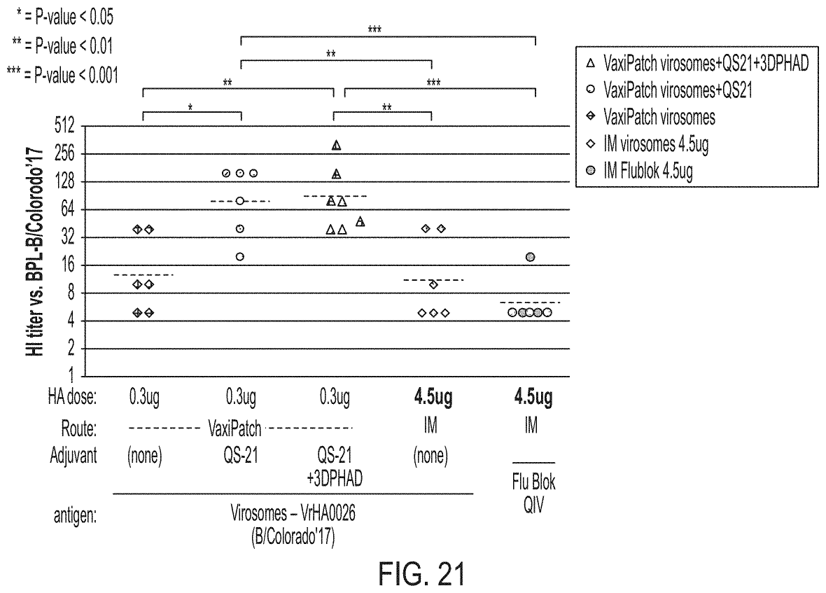

FIG. 21 shows Hemagglutination inhibition (HAI) titers to B/Colorado 2017 dot plot. To assess the quality of the immune responses elicited by Sprague-Dawley rat vaccinations, hemagglutination inhibition assays were performed against a cognate WHO standard antigen, BPL-inactivated B/Colorado/06/2017 influenza virions. For human sera, a 1:40 titer is considered to be protective in an HAI assay. Rat sera collected at day 28 post-vaccination was Kaolin treated to remove non-specific inhibitors of agglutination. Two-fold serial dilutions of treated post-immune sera were incubated with the BPL-inactivated antigen for 45 minutes at room temperature to allow binding. Human single-donor 0+red blood cells were added, and the ability of the immune sera to inhibit the agglutination reaction was scored. This dot plot shows the scores for all six animals per group, with darker shaded markers representing female animals. The Y-axis is a Log2 scale to reflect the dilution series. Geometric means for each group are noted with dashed lines. Statistical significance between groups is again shown, evaluated by one-way ANOVA followed by Tukey's HSD post-hoc tests.

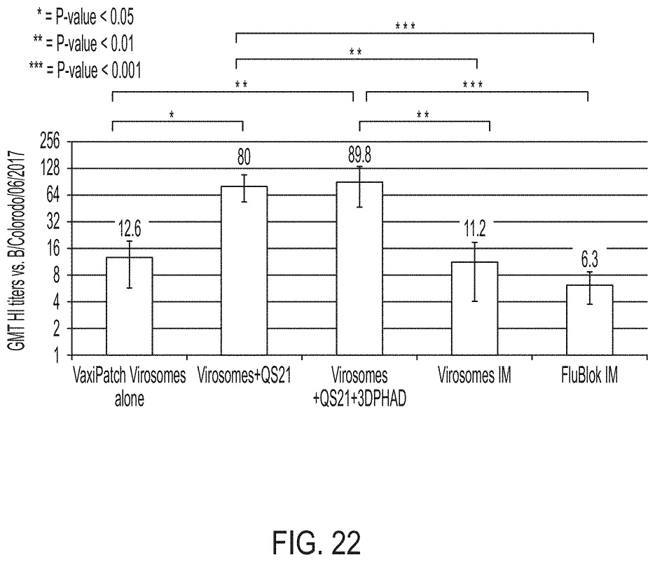

FIG. 22 shows a bar graph representation of HAI data. The same data set as shown in FIG. 21 is here expressed as a bar graph for clarity, with the geometric mean values plotted with error bars representing the standard error of the mean for the group size of 6 per set. Significant differences were observed between IM injections and VaxiPatch delivery of antigen, and between non-adjuvanted and adjuvanted VaxiPatch formulations.

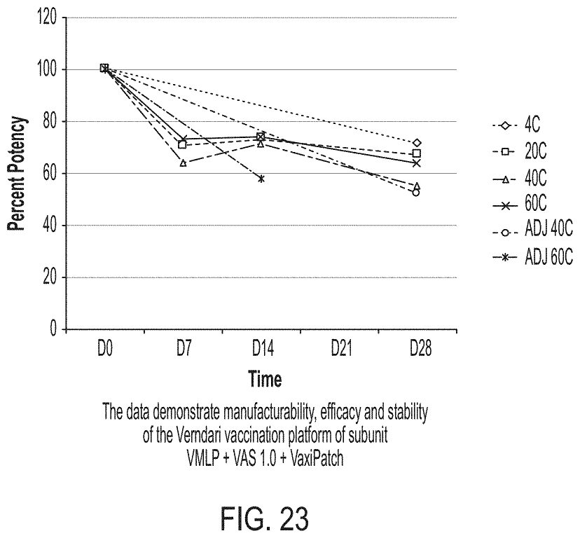

FIG. 23 shows VaxiPatch VMLP accelerated stability of antigen studies. To assess the stability of our vaccine formulations, 1 uL aliquots of our formulated print mixes (containing rHA antigen, dye, and trehalose) were packed under desiccation overnight to induce sugar glass formation. On the following day, samples were segregated to various storage temperatures for an accelerated aging study (4, 20, 40, or 60 degrees C.). At appropriate times, samples were removed and reconstituted in PBS, then subjected to potency testing using a single radial immunodiffusion assay (SRID) based on calibrated strain-specific reagents from NIBSC (Potters bar, UK). Values are expressed as a percentage of potency remaining as compared to the “day 0” controls, reconstituted at time of segregation. One adjuvanted preparation is also shown in this plot, including QS-21 and 3D-(6A)-PHAD. Strikingly, the majority of original HA potency is retained through 28 days, even at 60 degrees C.

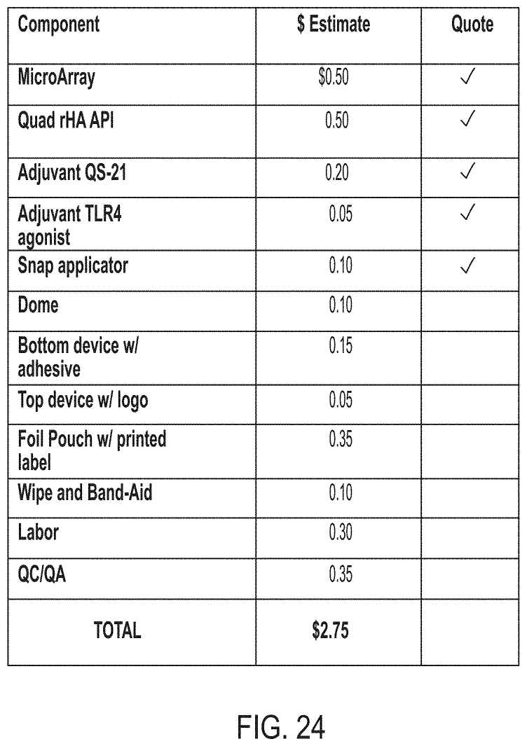

FIG. 24 shows that COGS are lower than industry average. For example, the influenza vaccine market today is approximately five billion dollars only in the developed world and three billion dollars in the United States. The CMS 2019/2020 AWP for classic flu is $20.34 and $56.00 for high dose. FluBlok® is $56.00. Shingrix® is $346.

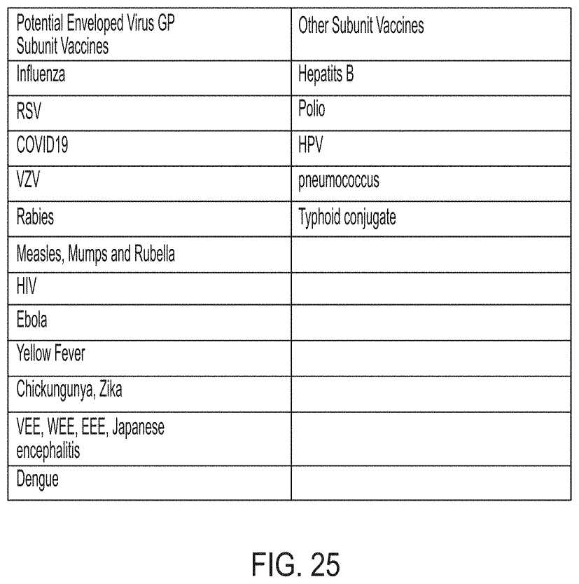

FIG. 25 shows an example chart with enveloped glycoprotein subunit vaccines. In some embodiments, a protein of a virus in FIG. 25 is included as an antigen in a VLP described herein. Any one of the viruses included in the figure may be included in the vaccine.

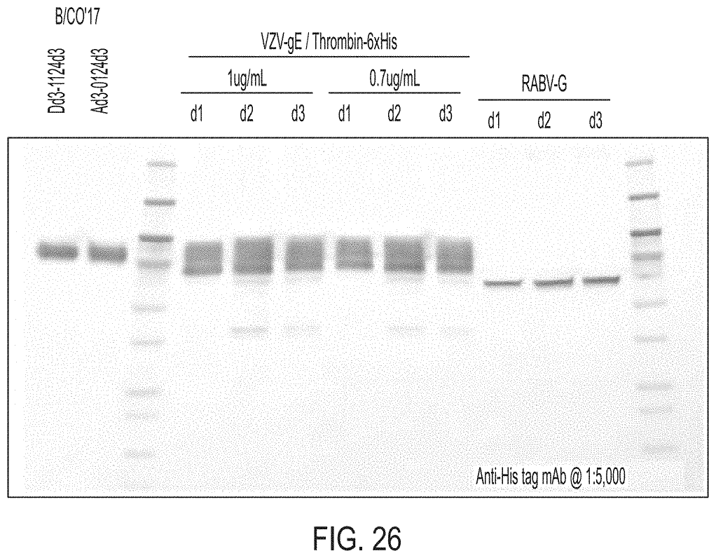

FIG. 26 shows a vaccine pipeline introduction. The approach to producing recombinant antigens is broadly applicable. Transfected cell lysate from two batches of influenza B rHAO are shown in the left lanes, as visualized by C-terminal 6xHis tags. The center lanes of this Western blot show an early timecourse of expression for the gE antigen from varicella-zoster virus (VZV-gE), the same protein which is used in the only currently-approved recombinant shingles vaccine. The right lanes show a timecourse of cells transfected with the G protein from rabies virus (RABV-G). Each of these viral glycoproteins bears a C-terminal His tag, allowing a broadly similar approach to initial detection and purification. While expression levels vary between the constructs, all can be made in the same mammalian high-density cell line (Expi293, in this example).

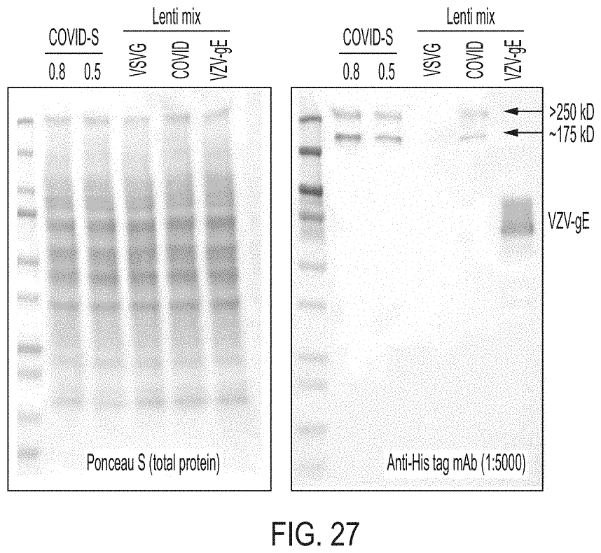

FIG. 27 shows an example COVID-S expression in ExpiCHO. The glycoprotein spike protein of SARS-CoV-2, the etiological agent of COVID-19, can also be expressed transiently in our system. Here it is shown ExpiCHO cell lysates at day 2 post-transfection with a His-tagged, full-length COVID-S construct. The panel on the right shows signal from the anti-His tag monoclonal antibody, indicating a specific band at ˜175 kD, consistent with a highly glycosylated 1273-aa protein. This band is absent from a parallel ExpiCHO flask lysate which was transfected with unrelated expression constructs (VSVG). The rightmost three lanes are from ExpiCHO cell cultures co-transfected with a lentiviral packaging plasmid and single-cycle vector bearing a constitutive GFP gene. These were matched with vesicular stomatitis virus G protein (VSV-G), COVID-S, or VZV-gE in order to generate pseudotyped, replication-deficient reporter virus particles. COVID-S and VZV-gE expression are detected in these samples on the basis of their C-terminal His tags, while the VSV-G control is not detected, as it lacks a His tag.

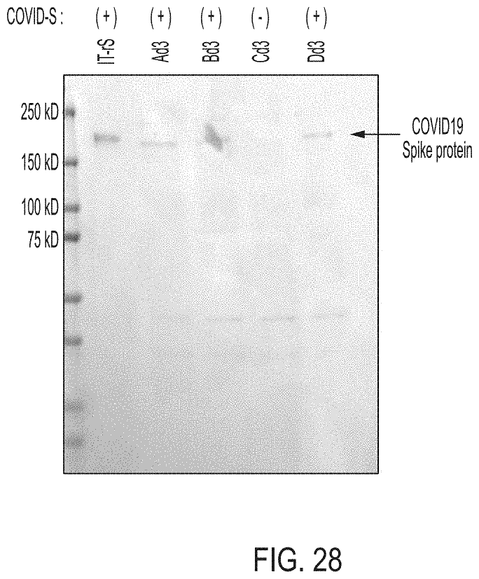

FIG. 28 shows an example COVID spike western blot that confirms the identity for recombinant COVID-S protein. In order to confirm that the 175-kD, His tag-reactive species was indeed the spike protein from SARS-CoV-2, Western blotting was performed using a commercial rabbit polyclonal antibody raised against a plasmid DNA vector expressing the COVID-19 spike protein (IT-002-030, Immune Technology Corp.). As a positive control, the commercial recombinant protein control was also run (IT-002-0032, Immune Technology Corp.). The purified protein control is in the left lane, labeled as “IT-rS”. An anti-rabbit secondary antibody visualized material at the expected —175 kD size for the purified protein control. Signal at a comparable size was present for three COVID-19 transfected cell lysates (Ad3, Bd3, Dd3), but was absent in a transfected cell lysate that did not receive the COVID-19 expression construct (Cd3). This served an important secondary indication of identity for our recombinant COVID-S antigen.

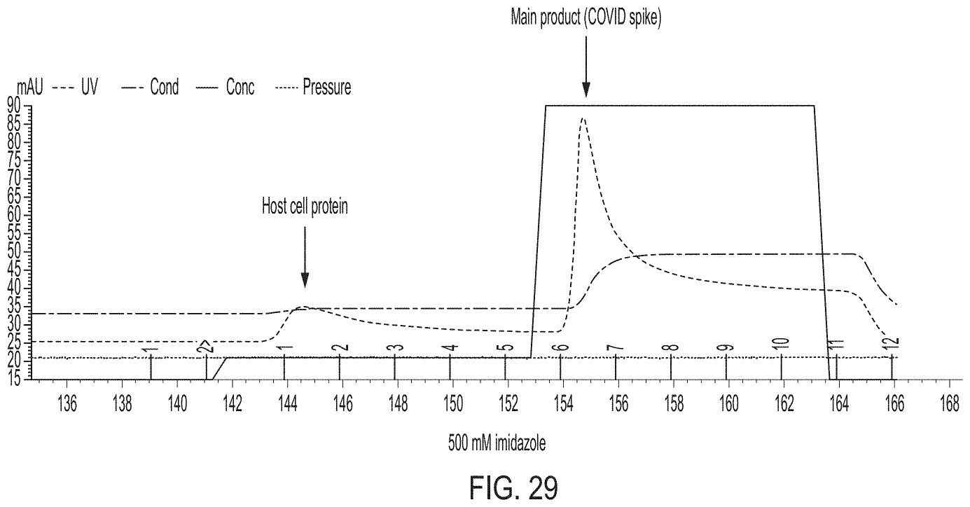

FIG. 29 shows a full-length spike purification with an elution profile of IMAC purification of COVID-S. The cell extract from approximately 30 mL of high-density ExpiCHO cell culture was applied to a HisTrap Crude FF 1-mL column (GE Healthcare), pre-equilibrated with buffer containing 0.5% LDAO. After detergent exchange into 1% octyl glucoside, a stepped gradient of imidazole was applied under constant 1% octyl glucoside to release loosely bound host cell protein, followed by release of the His-tagged recombinant protein. The blue dashed line trace indicates levels of released protein based on absorbance at 280 nm. The major peak at 154.5 mL contains the recombinant protein product. The nickel column purification allows for quick and highly specific purification of initial candidate vaccine material for preclinical testing but can be replaced by traditional protein chromatography methods which do not require addition of a heterologous epitope tag to the recombinant antigen product.

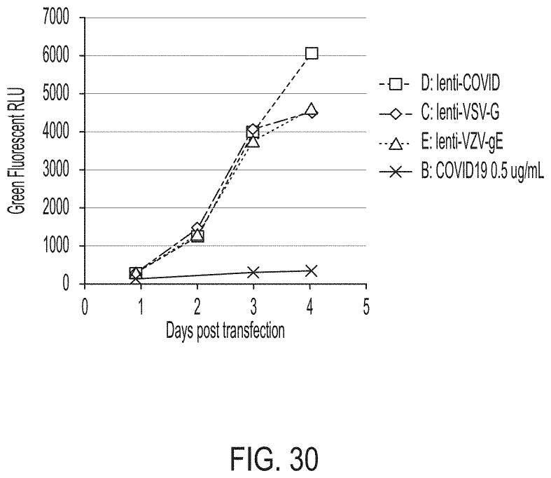

FIG. 30 shows a COVID-19 spike lentivirus pseudotype construction. key challenge of validating a novel vaccine is how to demonstrate potential efficacy. While IgG ELISA may model the magnitude of specific immune responses, it does not differentiate between antibodies which functionally inhibit the virus, and those which may bind non-essential (or structurally occluded) epitopes of the target protein. Neutralization assays, in which post-immune sera is tested for its ability to block virus entry into permissive cells in vitro, can be a powerful tool to predict efficacy in vivo. In order to avoid the need to use the highly infectious SARS-CoV-2 for such an assay, a pseudotype assay is being developed in which a replication-deficient reporter lentivirus is packaged using the COVID-S protein. If this pseudotype virus can transduce permissive cells in vitro, it should be possible to use it as a surrogate for authentic SARS-CoV-2 in neutralization assays. The lentiviral vector that was selected includes a constitutive GFP reporter. This plot shows fluorescence in transfected ExpiCHO cells over time, indicating activity of the lentiviral vector plasmid. Flask B, which was only transfected with the COVID-S construct (without the lentiviral vector), exhibits only background levels of fluorescence, while all three flasks transfected with packaging mixes demonstrate strong GFP signal by day 4 post-transfection.

### Example 12

### Generation of ACE-2

In some embodiments, ACE-2 was generated using a mammalian expression construct commissioned from ATUM Bio transiently transfected into expi293 cells. In such embodiments, the ectodomain of ACE-2 is secreted into the cell culture media. In such embodiments, three days post-transfection, cell culture supernatants were harvested and de-salted using PD-10 columns (GE-Health care, cat no 17-0851-01), and eluted in 100 mM NaCl, 20 mM Tris, pH 7.6. In such embodiments, the eluate was loaded onto an equilibrated HiTrap FF DEAE ion-exchange column, washed, and eluted with 200 mM NaCl, 20 mM Tris, pH 7.6.

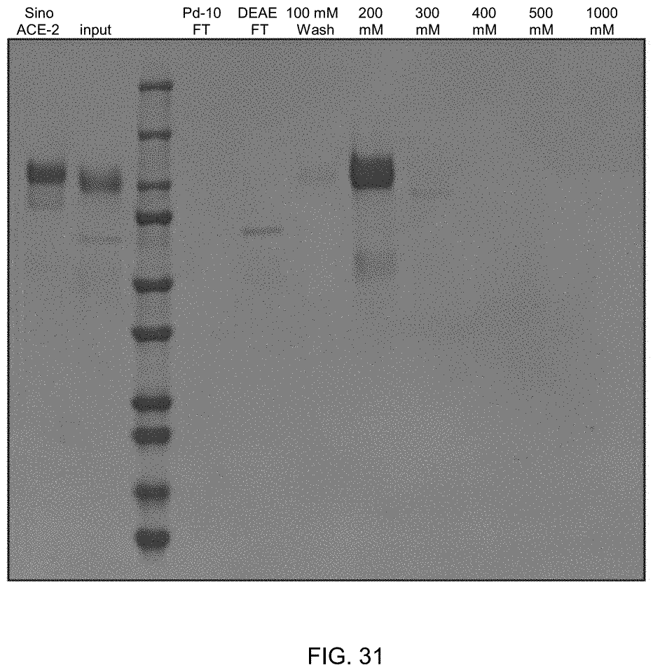

FIG. 31 depicts a Coomassie stained SDS-PAGE gel showing samples from a purification. As depicted, the first lane is commercial ACE-2 from Sino Biological (cat no 10108H08H20). To determine whether the ACE-2 protein, enzymatic activity was retained through purification and an enzymatic activity assay was performed using a fluorogenic substrate (R&D systems, cat no, ES007). A small peptide with a single letter amino acid sequence YVADAPK (SEQ ID NO: 17) was inserted between a highly fluorescent 7-methoxycoumarin (Mca) group and a 2,4-dinitrophenyl (Dnp) group that efficiently quenches the fluorescence of Mca by resonance energy transfer. ACE-2 cleaved the substrate between the Proline and the Lysine, and the increase in fluorescence was measured using a fluorescent plate reader with an excitation wavelength of 320 nm and emission of 405 nm.

The basic protocol for the assay was as follows:
1. Dilute the substrate to 40 uM in Assay buffer (1 M NaCl, 75 mM Tris, pH 7.5).
2. Add 50 uL of substrate to a black 96 well fluorescent assay plate for each well to be assayed.
3. Add 50 uL of sample diluted in the same Assay buffer.
4. Measure the fluorescence over time on a fluorescent plate reader.

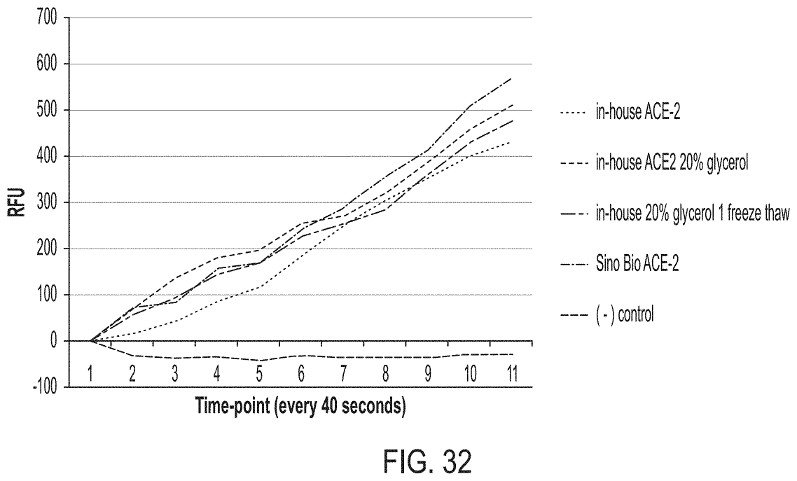

For the ACE-2 activity assay using a fluorogenic substrate, purified “in-house” ACE-2 was tested against the commercially available ACE-2 protein from Sino Biological. Whether the ACE-2 was active in 20% glycerol at 40C, and after 1 freeze-thaw cycle (2.5 hour incubation at −200C) for use internally was tested to determine storage conditions. All four samples appeared to have similar levels of activity, indicating that the purification methods used for ACE-2 did not have a detrimental effect on enzymatic activity. This also suggests that the ACE-2 can be stored in 20% glycerol and undergo at least 1 freeze thaw without losing a significant amount of activity. FIG. 32 depicts the levels of activity in the ACE-2 samples.

### Example 13

### Sandwich ELISA Development

The general protocol for the SARS-CoV-2-S potency assay is described below. In some embodiments, high-binding flat-bottom microtiter plates (Corning 3206) were coated overnight at 4° C. with ACE-2 (in-house purified ACE-2) at 2.5 μg/ml in PBS. The plates were then washed 3x with Tris-buffered saline (TBS) containing 0.05% TBST and blocked with 5% bovine serum albumin (BSA) in TBS for 2-4 h at room temperature. After one additional TBST wash, SARS-CoV-2-S protein in 1% BSA/TBST was added and incubated for 2 hours at room temperature, followed by four additional washes with TBST. Mouse-anti-SARS-CoV-2-S (GeneTex, cat no. GTX632604) was then added at 1:5000 in 1% BSA/TBST and incubated for 1 hour at room temperature. After an additional four washes, goat anti-mouse-HRP antibody (Jackson Labs, 715-035-150), at 1:5,000 in 1% BSA/TBST, was added and incubated for 1 hour at room temperature. After four final washes, 100 uL of TMB substrate was added, and incubated at room temperature for 30 minutes. The reaction was stopped by addition of 50 uL of 2N sulfuric acid. Resultant absorbance was then read at 450 nm on an automated microplate reader (AccuSkan FC, Fisher Scientific).

### Example 14

### SARS-CoV-2-S Potency Assay

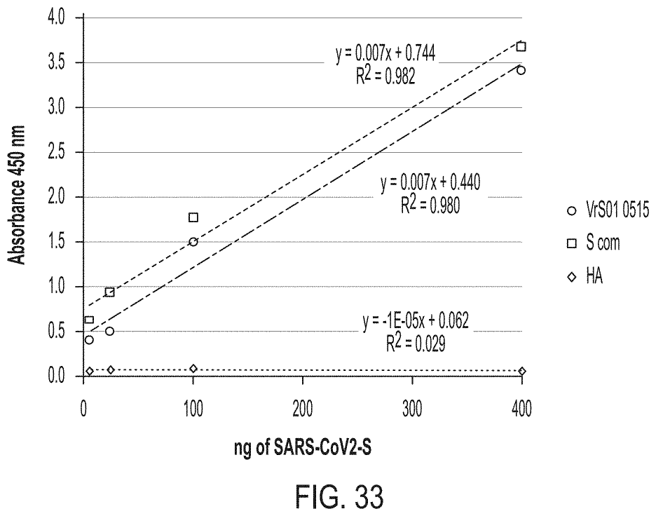

A potency assay was performed to compare the potency of VrS01 to a commercially available SARS-CoV-2-S from Immune-Tech (cat no. IT-002-032p). Hemagglutinin (HA) from in-house generatedB/Colorado '17 antigen was included as a negative control. The results were similar between the commercial (S com) and in-house SARS-CoV-2-S (VrS01 0515) proteins, and the HA had near zero binding at all concentrations tested. FIG. 33 depicts a linear regression of the data obtained or this experiment.

### Example 15

### Effects of Heat Stress on SARS-CoV-2-S Potency

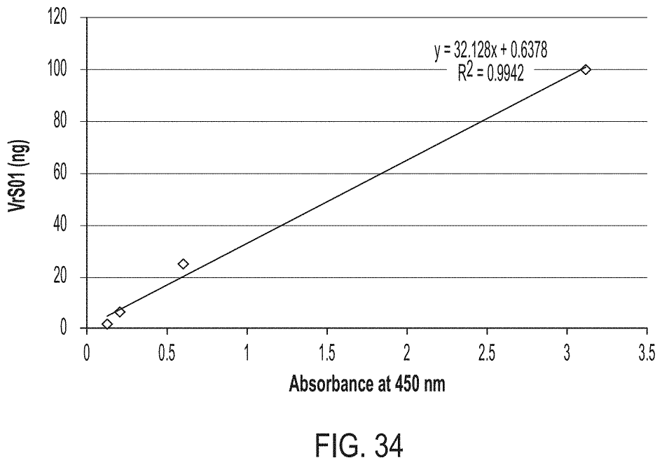

The ability of VrS01 to bind 250 ng of ACE-2 over four different concentrations (100, 25, 6.25, and 1.56 ng) was tested to establish a standard curve for the preliminary stability experiment described in more detail below. FIG. 34 depicts the standard curve.

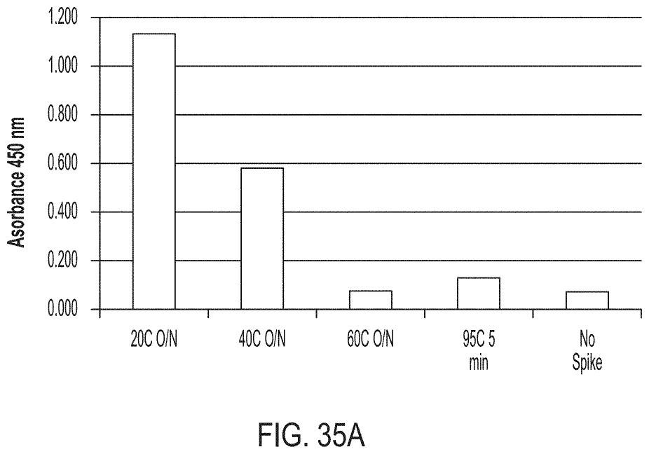

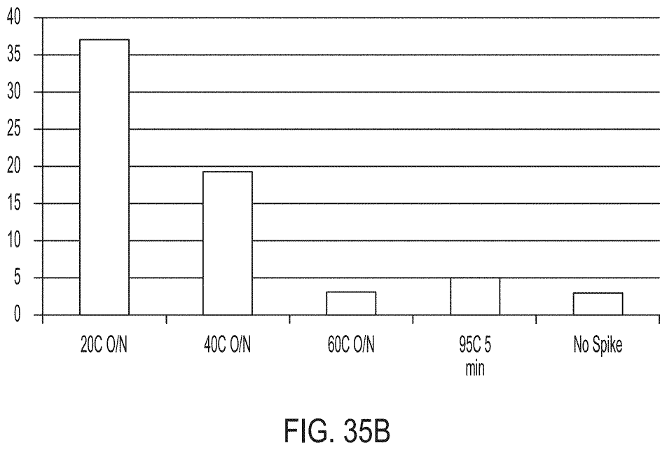

The stability of the VrS01 was tested at different temperatures (20, 40, and 60 degrees Celsius) and incubated the samples overnight. VrS01 was diluted in 1% BSA in TBST at a concentration of 0.5 ng/uL, so that when 100 ul of these samples was added to the ACE2 coated well 50 ng was added. A sample at 950C was also boiled for 5 minutes. FIG. 35A depicts data obtained in this experiment. FIG. 35B depicts the amount of potent VrS01 remaining determined based on converting the absorbance values using the standard curve depicts in FIG. 34.

The percent potency for each condition as a percentage can calculated by dividing the potent VrS01 by the amount of VrS01 added to the well and multiplying by 100. Values were calculated as shown in Table 2.

TABLE 2
calculated potency values

| | |
|---|---|
| 20 C. O/N | 74.0% |
| 40 C. O/N | 38.6% |
| 60 C. O/N | 6.3% |
| 95 C. 5 min | 9.8% |
| No Spike | 6.0% |

### Example 16

### VMLP Bound VrS01 Formulated with Adjuvant:

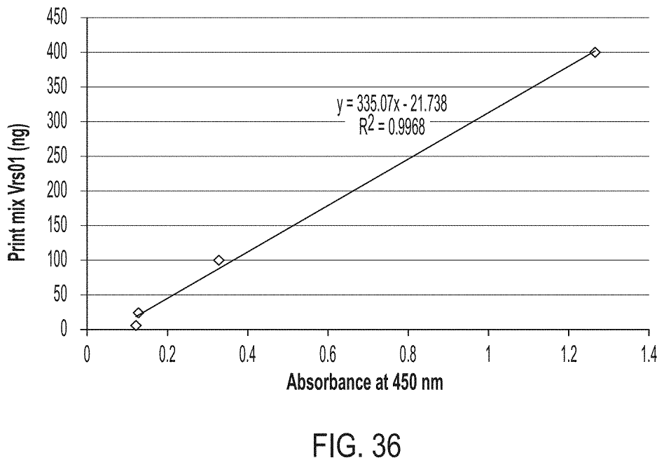

The ability of VMLPs that had been formulated into “print mix” (e.g. a formulation used for printing VaxiPatch arrays) to bind ACE-2 was tested by adding 400, 100, 25, or 6.25 ng of SARS-CoV-2-S to a well containing 250 ng of ACE-2. A linear relationship between the amount of VMLPs and the absorbance measured in the well was observed. The values observed for the amount of binding to ACE-2 were lower than for the “free” protein. This could be due to lower potency through formulation or differences in the kinetics of binding when SARS-CoV-2-S is incorporated into a VMLP. FIG. 36 depicts a linear regression for “print mix” VMLPs.

TABLE 3
linear regression for “print mix” VMLPs

| Absorbance | VMLP (ng) |
|---|---|
| 1.265 | 400 |
| 0.3285 | 100 |
| 0.129 | 25 |
| 0.1225 | 6.25 |

### Example 17

### pH sensitivity of ACE-2/SARS-CoV-2-S Binding

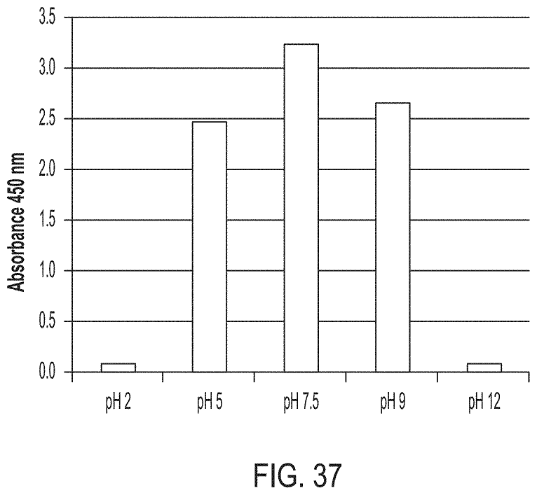

The pH sensitivity of the ACE-2/VrS01 binding was tested. The experiment was performed by pre-incubating 250 ul of 1 ng/uL VrS01 at the pH levels of 2, 5, 7.5, 9, or 12. The pH was adjusted with either NaOH or HCl and measured using strips of pH paper. 100 ul of each sample was loaded in duplicate onto a plate coated with 250 ng of ACE-2 and the absorbance was measured at 450 nm. The amount of ACE-2 binding appeared to be slightly reduced at the pH of 5 and 9, but was only slightly above background at pH of 2 and 12. FIG. 37 depicts a graph of the ACE-2 binding at different pH levels.

TABLE 4
ACE-2 binding at different pH levels

| pH value | Absorbance |
|---|---|
| pH 2 | 0.091 |
| pH 5 | 2.486 |
| pH 7.5 | 3.234 |
| pH 9 | 2.668 |
| pH 12 | 0.074 |

### Example 18

### Inhibition of ACE-2 Binding with a Polyclonal Antibody to the S1 Subunit of SARS-CoV-2-S

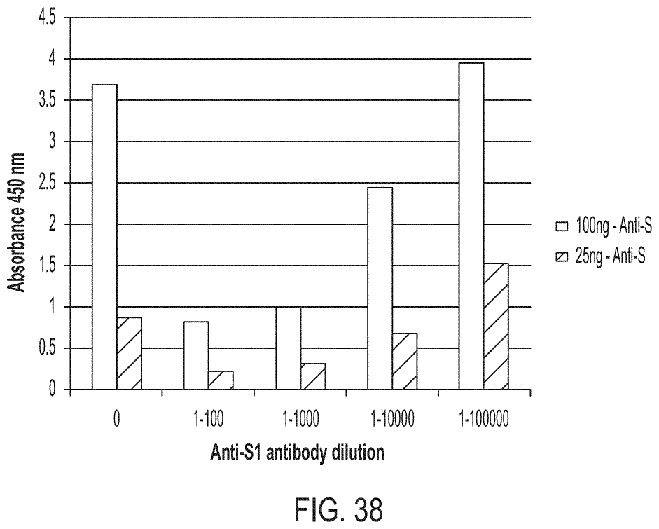

In preparation and anticipation of performing analysis on sera of vaccinated animal models, a test was performed on the ability of a commercially available polyclonal rabbit antibody to the S1 subunit of SARS-CoV-2-S to inhibit the binding interaction with ACE-2. The binding assay was performed as described in the design above, except that while the ACE-2 coated plate was in blocking solution, 1 ng/ul or 0.25 ng/ul was incubated in-house SARS-CoV-2-S with multiple dilutions of the commercial antibody. After addition of the antibody, the samples were incubated at 37 degrees Celsius for 2 hours. The samples were loaded in duplicate onto wells coated with 250 ng of ACE-2 and the absorbance was determined at 450 nm. FIG. 38 depicts a bar graph with a plot of the average absorbance.

The polyclonal antibody was able to effectively inhibit the binding of SARS-CoV-2-S to ACE-2 when using a dilution of 1:100 or 1:1000, and there was partial inhibition of the binding at the dilution of 1:10,000. This result was true for both the 100 and 25 ng SARS-CoV-2-S conditions.

TABLE 5
summarizing the absorbance values

| Antibody dilution | 100 ng - Anti-S | 25 ng - Anti-S |
|---|---|---|
| 0 | 3.68 | 0.862 |
| 1-100 | 0.831 | 0.222 |
| 1-1000 | 0.989 | 0.324 |
| 1-10000 | 2.447 | 0.686 |
| 1-100000 | 3.960 | 1.537 |

### Example 19

### Recombinant SARS-CoV-2 Spike Protein Purification and VMLP Formulation

SARS-CoV-2 (Wuhan'19) recombinant spike (rS) was designed with a thrombin cleavage site leading to a 6×HIS tag at the C-terminus of the ORF, designated as VrS01. Once cleaved by thrombin, the rS protein product would only include four residual amino acids (Leu-Val-Pro-Arg) appended to the wild-type sequence. The native multibasic S1/S2 cleavage site for the S protein was left intact. The amino acid sequence of the synthetic construct was in accordance with SEQ ID NO: 30. Note: the underlined sequence represents the synthetic thrombin cleavage site, while the last six amino acids are the C-terminal 6×His tag.

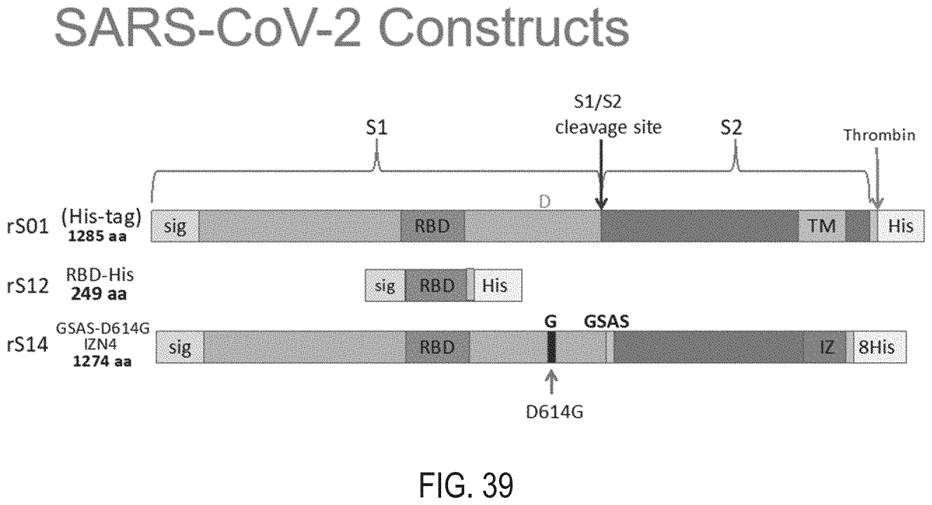

FIG. 39 shows a summary diagram of this construct (VrS01), as compared to a His-tagged RBD alone (VrS12) and a full-length secretable ectodomain construct bearing D614G and furin site mutations (VrS14). ATUM bio was used as a synthesis vendor. The pD2610-v10 plasmid backbone was used. This vector was designed for high-level transient expression and bears a Kanamycin resistance gene for bacterial selection. After sequence optimization for CHO cells, the DNA sequence was in accordance with SEQ ID NO: 31 (VrS01 DNA sequence, codon optimized for mammalian expression).

ExpiCHO-S cells (Fisher) were expanded at passage P8 to an E1000 flask, from a vial frozen at P1. This expansion culture attained a density of 8.66 ×106. One E1000 flask was prepared with 1200M cells in 200 mL of ExpiCHO Expression media. Transfections were performed using 160 uL of plasmid stock at 1 ug/mL, by means of an ExpiFectamine CHO transfection Kit (Fisher). At 24 hours post-transfection, enhancer and feed reagents were added to transfection cultures, and a temperature shift to 32° C. was applied. Daily density and viability evaluations were made by trypan blue exclusion using 0.4 mL of suspension culture. Washed cell pelleted were banked at days 2 and 3 post-transfection, with the day 3 cell pellet used for purification of VrS01 for pilot immunogenicity tests.

Frozen cell pellets were resuspended in lx PBS and subjected to a 20 minute centrifugation at 4,000×g to remove some soluble cellular protein. Lysis of VrS01-bearing cell pellets was then performed in 50 mM HEPES buffer (pH 7.5), 500 mM NaCl, 2 mM MgCl₂ (to support Benzonase activity) and 2% LDAO detergent (n-Dodecyl-N,N-Dimethylamine-N-Oxide, Anatrace). Benzonase treatment (200 U) was applied for 10 minutes at room temperature, followed by 1 hour of gentle rotation at 4° C. Two rounds of centrifugation were applied to clear extracts of insoluble cell debris; a first spin at 4,000×g for 20 minutes, followed by a second spin at 10,000×g for 40 minutes. Cleared extracts were then mixed with pre-equilibrated Capto Lentil Lectin resin (Cytiva) and rotated for 4-6 hours at 4° C. Bound resin was washed with wash buffer (50 mM HEPES, 500 mM NaCl, 0.5% LDAO; pH 7.5) prior to packing into gravity columns for additional washes. An on-column detergent exchange was performed into 1% octyl glucoside, 50 mM HEPES, 500 mM NaCl (pH 7.5), followed by elution with 300 mM α-D methyl-glucoside. This eluate was then supplemented with imidazole to 5 mM and applied to a 1-mL HisTrap FF crude column via a syringe pump. Eluate was passed over the column three times prior to washing with a 5 mM imidazole, 1% OG solution, and final elution in 500 mM imidazole. The resulting OG-micellized VrS01 was concentrated on an Amicon Ultra-15 30K diafiltration column and dialyzed against VDB-OG (10 mM NaP, 140 mM NaCl, 1% octyl glucoside; pH 7.2) to remove imidazole. VrS01 was then quantified by BCA assay (Pierce) and purity confirmed by SDS-PAGE analysis.

VMLPs were formed with VrS01 by the same method described in Example 6. For reconstitution as seVLPs, 0.65 mg of lipids (phosphatidyl choline (50 mg/ml), and plant cholesterol (20 mg/ml) in a ratio of 2:1) were dissolved in 130 μl 10% OG. 200 ug of OG-micellized VrS01 was then added to the dissolved lipids and the total volume was made up to 0.65 mL, giving an end concentration of ˜4% OG. The sample was dialyzed against numerous changes of small volumes (26 ml) of PBS for 24 hours at 4° C. The sample was then dialyzed against 4×32 ml PBS over 24 hours. The sample was then transferred to 4×2 L over 48 hours, to remove OG. All dialysis steps were against VDB (10 mM NaP, 140 mM NaCl; pH 7.2) at 4° C. seVLPs were 230-250 nm in average diameter as determined by dynamic light scattering (DLS) using a Malvern Zetasizer-NS, as compared to empty DOPC/chol liposomes prepared in parallel (no VrS01 incorporation), which had average diameters of 190-200 nm.

### Example 20

### Immunogenicity of VrS01 seVLPs in Sprague-Dawley Rats

VaxiPatch arrays were prepared as described in Examples 7 and 8, from print mixes formulated to contain either 100 or 500 ng of seVLP-VrS01, along with liposomal adjuvant at a dose of 500 ng QS-21 and 500 ng 3D(6-Acyl)-PHAD per patch, with 0.5% (w/v) FD&C No.1 blue dye for visualization. These were applied to 8 Sprague Dawley rats (4 males, 4 females) in the same manner as Example 8. Briefly, Sprague-Dawley rats, with hair previously removed, were treated with arrays utilizing 5 minute direct pressure to the midline of the back while under isofluorane anesthesia. Serum was collected by saphenous vein bleeds at 2, 3, and 4 weeks post-treatment. On week 5, animals from both groups received an additional VaxiPatch boost by the same method, consisting of 175 ng seVLP-VrS01 plus adjuvant as described above (500 ng QS-21, 500 ng 3D(6-Acyl)-PHAD. Sera was again drawn and tested 2 weeks post-boost.

Specific IgG responses in the sera were evaluated by ELISA assay on plates coated overnight at 4° C. in 100 mM carbonate buffer with full-length rS (Immune Tech, IT-003-032p) at 0.5 μg/mL. Five-fold serial dilutions from 1:100 to 1:12,500 were tested, using an HRP-conjugated polyclonal goat antibody against rat IgG for detection (Jackson labs, 112-035-143). Positivity was assigned based on signal in excess of twice the blank wells of the plate, and used to assign endpoint titers.

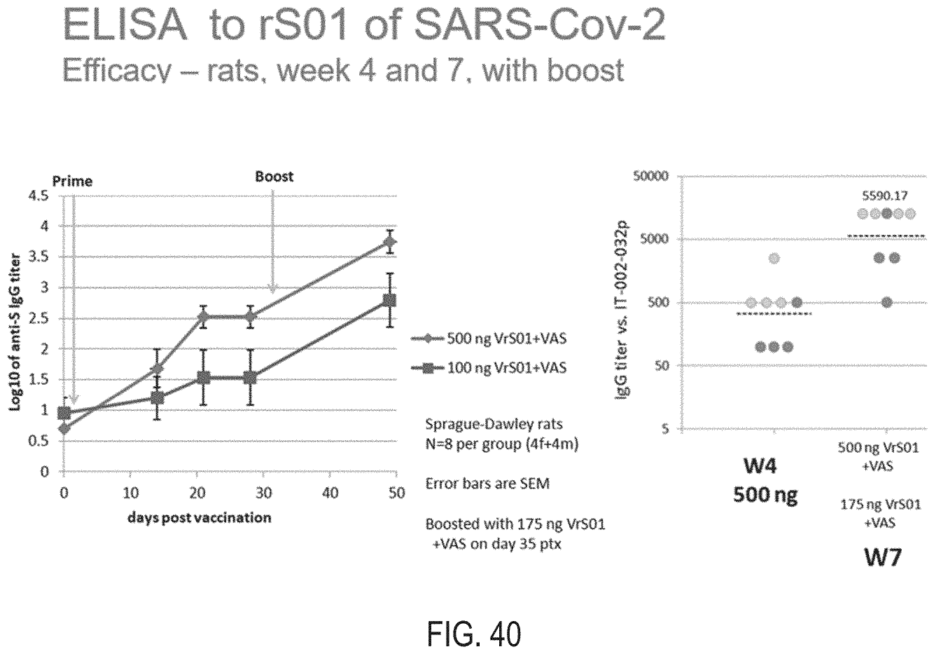

FIG. 40 summarizes specific IgG responses to VrS01 in SD rats. The left panel shows the time-course of the VrS01 treatment groups based on the log10 of their assigned endpoint titers, with arrows indicating the timing of both vaccination treatments. Error bars represent SEM (n=8 per group). The right panel compares endpoint titers from individual animals within the 500 ng treatment group, at 4 weeks post initial vaccination, and 2 week post-boost. Markers in darker shade represent male animals. Dashed lines indicate GMT titers for each group.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCES

| # | SINGLE LETTER AMINO ACID SEQUENCE | | ANNOTATION |
|---|---|---|---|
| 1 | MNPNQKIITIGSTCMTIGMANLILQIGNIISIWVSHSIQIGNQS | | N1 NA sequence |
| | QIETCNQSVITYENNTWVNQTYVNISNTNFAARQSVASVKL | | for |
| | AGNSSLCPVSGWAIYSKDNSVRIGSKGDVFVIREPFISCSPLE | | A/Brisbane/02/2018, |
| | CRTFFLTQGALLNDKHSNGTIKDRSPYRTLMSCPIGEVPSPY | | accession |
| | NSRFESVAWSASACHDGTNWLTIGISGPDSGAVAVLKYNGI | | number |
| | ITDTIKSWRNNILRTQESECACVNGSCFTIMTDGPSDGQASY | | EPI1322978 |
| | KIFRIEKGKIIKSVEMKAPNYHYEECSCYPDSSEITCVCRDN | | (GISAID EpiFlu |
| | WHGSNRPWVSFNQNLEYQMGYICSGVFGDNPRPNDKTGS | | database, |
| | CGPVSSNGANGVKGFSFKYGNGVWIGRTKSISSRKGFEMI | | www.gisaid.org/) |
| | WDPNGWTGTDNKFSIKQDIVGINEWSGYSGSFVQHPELTG | | |
| | LDCIRPCFWVELIRGRPEENTIWTSGSSISFCGVDSDTVGWS | | |
| | WPDGAELPFTIDK | | |
| 2 | MNPNQKIITIGSVSLTISTICFFMQIAILITTVTLHFKQYEFNSP | | N2 NA sequence |
| | PNNQVMLCEPTIIERNITEIVYLTNTTIEREICPKPAEYRNWS | | for |
| | KPQCGITGFAPFSKDNSIRLSAGGDIWVTREPYVSCDPDKC | | A/Kansas/14/2017, |
| | YQFALGQGTTINNVHSNNTARDRTPHRTLLMNELGVPFHL | | accession number |
| | GTKQVCIAWSSSSCHDGKAWLHVCITGDDKNATASFIYNG | | EPI1146344 |
| | RLVDSVVSWSKDILRTQESECVCINGTCTVVMTDGNATGK | | (GISAID EpiFlu |
| | ADTKILFIEEGKIVHTSKLSGSAQHVEECSCYPRYPGVRCVC | | database, |
| | RDNWKGSNRPIVDINIKDHSIVSSYVCSGLVGDTPRKTDSSS | | www.gisaid.org/) |
| | SSHCLNPNNEKGGHGVKGWAFDDGNDVWMGRTINETSRL | | |
| | GYETFKVVEGWSNPKSKLQINRQVIVDRGDRSGYSGIFSVE | | |
| | GKSCINRCFYVELIRGRKEETEVLWTSNSIVVFCGTSGTYGT | | |
| | GSWPDGADLNLMHI | | |
| 3 | MLPSTIQTLTLFLTSGGVLLSLYVSASLSYLLYSDILLKFSPT | | NA sequence for |
| | EITAPTMPLDCANASNVQAVNRSATKGVTLLLPEPEWTYP | | B/Colorado/06/2017, |
| | RLSCPGSTFQKALLISPHRFGETKGNSAPLIIREPFVACGPNE | | accession |
| | CKHFALTHYAAQPGGYYNGTRGDRNKLRHLISVKLGKIPT | | number |
| | VENSIFHMAAWSGSACHDGKEWTYIGVDGPDNNALLKVK | | EPI969379 |
| | YGEAYTDTYHSYANNILRTQESACNCIGGNCYLMITDGSAS | | (GISAID EpiFlu |
| | GVSECRFLKIREGRIIKEIFPTGRVKHTEECTCGFASNKTIEC | | database, |
| | ACRDNRYTAKRPFVKLNVETDTAEIRLMCTDTYLDTPRPN | | www.gisaid.org/) |
| | DGSITGPCESDGDKGSGGIKGGFVHQRMKSKIGRWYSRTM | | |
| | SQTERMGMGLYVKYGGDPWADSDALAFSGVMVSMKEPG | | |
| | WYSFGFEIKDKKCDVPCIGIEMVHDGGKETWHSAATAIYC | | |
| | LMGSGQLLWDTVTGVDMAL | | |
| 4 | MLPSTIQTLTLFLTSGGVLLSLYVSASLSYLLYSDILLKFSRT | | NA sequence for |
| | EVTAPIMPLDCANASNVQAVNRSATKGVTPLLPEPEWTYP | | B/Phuket/3073/2013, |
| | RLSCPGSTFQKALLISPHRFGETKGNSAPLIIREPFIACGPKEC | | accession |
| | KHFALTHYAAQPGGYYNGTREDRNKLRHLISVKLGKIPTV | | number |
| | ENSIFHMAAWSGSACHDGREWTYIGVDGPDSNALLKIKYG | | EPI1349898 |
| | EAYTDTYHSYAKNILRTQESACNCIGGDCYLMITDGPASGI | | (GISAID EpiFlu |
| | SECRFLKIREGRIIKEIFPTGRVKHTEECTCGFASNKTIECAC | | database, |
| | RDNSYTAKRPFVKLNVETDTAEIRLMCTKTYLDTPRPNDGS | | www.gisaid.org/) |
| | ITGPCESDGDEGSGGIKGGFVHQRMASKIGRWYSRTMSKT | | |
| | KRMGMGLYVKYDGDPWTDSEALALSGVMVSMEEPGWYS | | |
| | FGFEIKDKKCDVPCIGIEMVHDGGKTTWHSAATAIYCLMG | | |
| | SGQLLWDTVTGVNMTL | | |
| 5 | MKAILVVLLYTFTTANADTLCIGYHANNSTDTVDTVLEKN | | HA0 sequence for |
| | VTVTHSVNLLEDKHNGKLCKLGGVAPLHLGKCNIAGWILG | | A/Brisbane/02/2018, |
| | NPECESLSTARSWSYIVETSNSDNGTCYPGDFINYEELREQL | | accession |
| | SSVSSFERFEIFPKTSSWPNHDSNKGVTAACPHAGAKSFYK | | number |
| | NLIWLVKKGNSYPKLNQTYINDKGKEVLVLWGIHHPPTTA | | EPI1322979 |
| | DQQSLYQNADAYVFVGTSRYSKKFKPEIATRPKVRDREGR | | (GISAID EpiFlu |
| | MNYYWTLVEPGDKITFEATGNLVVPRYAFTMERNAGSGIII | | database, |
| | SDTPVHDCNTTCQTAEGAINTSLPFQNVHPVTIGKCPKYVK | | www.gisaid.org/) |
| | STKLRLATGLRNVPSIQSRGLFGAIAGFIEGGWTGMVDGW | | |
| | YGYHHQNEQGSGYAADLKSTQNAIDKITNKVNSVIEKMNT | | |
| | QFTAVGKEFNHLEKRIENLNKKVDDGFLDIWTYNAELLVL | | |
| | LENERTLDYHDSNVKNLYEKVRNQLKNNAKEIGNGCFEFY | | |
| | HKCDNTCMESVKNGTYDYPKYSEEAKLNREKIDGVKLEST | | |
| | RIYQILAIYSTVASSLVLVVSLGAISFWMCSNGSLQCRICI | | |
| 6 | MKTIIALSCILCLVFAQKIPGNDNSTATLCLGHHAVPNGTIV | | HA0 sequence for |
| | KTITNDRIEVTNATELVQNSSIGEICDSPHQILDGENCTLIDA | | A/Kansas/14/2017, |
| | LLGDPQCDGFQNKKWDLFVERNKAYSNCYPYDVPDYASL | | accession number |
| | RSLVASSGTLEFNNESFNWAGVTQNGTSSSCIRGSKSSFFSR | | EPI1146345 |
| | LNWLTHLNSKYPALNVTMPNNEQFDKLYIWGVHHPGTDK | | (GISAID EpiFlu |
| | DQISLYAQSSGRITVSTKRSQQAVIPNIGSRPRIRDIPSRISIY | | database, |
| | WTIVKPGDILLINSTGNLIAPRGYFKIRSGKSSIMRSDAPIGK | | www.gisaid.org/) |
| | CKSECITPNGSIPNDKPFQNVNRITYGACPRYVKQSTLKLAT | | |
| | GMRNVPERQTRGIFGAIAGFIENGWEGMVDGWYGFRHQN | | |
| | SEGRGQAADLKSTQAAIDQINGKLNRLIGKTNEKFHQIEKE | | |
| | FSEVEGRIQDLEKYVEDTKIDLWSYNAELLVALENQHTIDL | | |
| | TDSEMNKLFEKTKKQLRENAEDMGNGCFKIYHKCDNACM | | |
| | GSIRNGTYDHNVYRDEALNNRFQIKGVELKSGYKDWILWI | | |
| | SFAISCFLLCVALLGFIMWACQKGNIRCNICI | | |
| 7 | MKTIIALSYILCLVFAQKIPGNDNSTATLCLGHHAVPNGTIV | | HA0 sequence for |
| | KTITNDRIEVTNATELVQNSSIGEICDSPHQILDGENCTLIDA | | B/Colorado/06/2017, |
| | LLGDPQCDGFQNKKWDLFVERSKAYSNCYPYDVPDYASL | | accession |
| | RSLVASSGTLEFKNESFNWTGVTQNGKSSACIRGSSSSFFSR | | number (GISAID |
| | LNWLTHLNYTYPALNVTMPNKEQFDKLYIWGVHHPGTDK | | EpiFlu database, |
| | DQIFLYAQSSGRITVSTKRSQQAVIPNIGSRPRIRDIPSRISIY | | EPI941626, |
| | WTIVKPGDILLINSTGNLIAPRGYFKIRSGKSSIMRSDAPIGK | | www.gisaid.org/) |
| | CKSECITPNGSIPNDKPFQNVNRITYGACPRYVKHSTLKLAT | | |
| | GMRNVPEKQTRGIFGAIAGFIENGWEGMVDGWYGFRHQN | | |
| | SEGRGQAADLKSTQAAIDQINGKLNRLIGKTNEKFHQIEKE | | |
| | FSEVEGRVQDLEKYVEDTKIDLWSYNAELLVALENQHTID | | |
| | LTDSEMNKLFEKTKKQLRENAEDMGNGCFKIYHKCDNACI | | |
| | GSIRNETYDHNVYRDEALNNRFQIKGVELKSGYKDWILWIS | | |
| | FAISCFLLCVALLGFIMWACQKGNIRCNICI | | |
| 8 | MKAIIVLLMVVTSNADRICTGITSSNSPHVVKTATQGEVNV | | HA0 sequence for |
| | TGVIPLTTTPTKSYFANLKGTRTRGKLCPDCLNCTDLDVAL | | B/Phuket/3073/2013, |
| | GRPMCVGTTPSAKASILHEVRPVTSGCFPIMHDRTKIRQLPN | | accession |
| | LLRGYEKIRLSTQNVIDAEKAPGGPYRLGTSGSCPNATSKIG | | number (GISAID |
| | FFATMAWAVPKDNYKNATNPLTVEVPYICTEGEDQITVWG | | EpiFlu database, |
| | FHSDNKTQMKSLYGDSNPQKFTSSANGVTTHYVSQIGDFP | | EPI1349899, |
| | DQTEDGGLPQSGRIVVDYMMQKPGKTGTIVYQRGVLLPQK | | www.gisaid.org/) |
| | VWCASGRSKVIKGSLPLIGEADCLHEEYGGLNKSKPYYTG | | |
| | KHAKAIGNCPIWVKTPLKLANGTKYRPPAKLLKERGFFGAI | | |
| | AGFLEGGWEGMIAGWHGYTSHGAHGVAVAADLKSTQEAI | | |
| | NKITKNLNSLSELEVKNLQRLSGAMDELHNEILELDEKVDD | | |
| | LRADTISSQIELAVLLSNEGIINSEDEHLLALERKLKKMLGPS | | |
| | AVDIGNGCFETKHKCNQTCLDRIAAGTFNAGEFSLPTFDSL | | |
| | NITAASLNDDGLDNHTILLYYSTAASSLAVTLMLAIFIVYM | | |
| | VSRDNVSCSICL | | |
| 9 | MSLLTEVETHTRSEWECRCSGSSDPLVIAANIIGILHLILWIT | | M2 sequence for |
| | DRLFFKCIYRRFKYGLKRGPSTEGVPESMREEYQQEQQSAV | | A/Brisbane/02/2018, |
| | DVDDGHFVNIELE | | accession |
| | | | number |
| | | | EPI1312561 |
| | | | (GISAID EpiFlu |
| | | | database, |
| | | | www.gisaid.org/) |
| 10 | MSLLTEVETPIRNEWGCRCNDSSDPLIVAANIIGILHLILWIL | | M2 sequence for |
| | DRLFFKCVCRLFKHGLKRGPSTEGVPESMREEYRKEQQNA | | A/Kansas/14/2017, |
| | VDADDSHFVSIELE | | accession number |
| | | | EPI1146340 |
| | | | (GISAID EpiFlu |
| | | | database, |
| | | | www.gisaid.org/) |
| 11 | MLEPFQILTICSFILSALHFMAWTIGHLNQIKRGINMKIRIKG | | B2M sequence for |
| | PNKETITREVSILRHSYQKEIQAKETMKEVLSDNMEVLNDH | | B/Colorado/06/2017, |
| | IIIEGLSAEEIIKMGETVLEIEELH | | accession |
| | | | number |
| | | | EPI969376 |
| | | | (GISAID EpiFlu |
| | | | database, |
| | | | www.gisaid.org/) |
| 12 | MFEPFQILSICSFILSALHFMAWTIGHLNQIKRGVNMKIRIKG | | B2M sequence for |
| | PNKETINREVSILRHSYQKEIQAKEAMKEVLSDNMEVLSDH | | B/Phuket/3073/2013, |
| | IVIEGLSAEEIIKMGETVLEVEESH | | accession |
| | | | number |
| | | | EPI1349894 |
| | | | (GISAID EpiFlu |
| | | | database, |
| | | | www.gisaid.org/) |
| 13 | MNNATFNYTNVNPISHIRGSIIITICVSFIIILTILGYIAKILTNR | | NB sequence for |
| | NNCTNNAIGLCKRIKCSGCEPFCNKRGDTSSPRTGVDIPAFI | | B/Colorado/06/2017, |
| | LPGLNLSESTPN | | accession |
| | | | number |
| | | | EPI969379 |
| | | | (GISAID EpiFlu |
| | | | database, |
| | | | www.gisaid.org/) |
| 14 | MNNATFNYTNVNLISHIRGSVIITICVSFIVILTIFGYIAKIFTN | | NB sequence for |
| | RSNCTNNAIGLCKRIKCSGCEPFCNKRGDTSSPRTGVDVPSF | | B/Phuket/3073/2013, |
| | ILPGLNLSESTPN | | accession |
| | | | number |
| | | | EPI1349898 |
| | | | (GISAID EpiFlu |
| | | | database, |
| | | | www.gisaid.org/) |
| 15 | MKAIIVLLMVVTSSADRICTGITSSNSPHVVKTATQGEVNV | | B/CO′17 rHA |
| | TGVIPLTTTPTKSHFANLKGTETRGKLCPKCLNCTDLDVAL | | |
| | GRPKCTGKIPSARVSILHEVRPVTSGCFPIMHDRTKIRQLPN | | |
| | LLRGYEHVRLSTHNVINAEGAPGGPYKIGTSGSCPNITNGN | | |
| | GFFATMAWAVPDKNKTATNPLTIEVPYVCTEGEDQITVWG | | |
| | FHSDNETQMAKLYGDSKPQKFTSSANGVTTHYVSQIGGFP | | |
| | NQTEDGGLPQSGRIVVDYMVQKSGKTGTITYQRGILLPQK | | |
| | VWCASGRSKVIKGSLPLIGEADCLHEKYGGLNKSKPYYTG | | |
| | EHAKAIGNCPIWVKTPLKLANGTKYRPPAKLLKERGFFGAI | | |
| | AGFLEGGWEGMIAGWHGYTSHGAHGVAVAADLKSTQEAI | | |
| | NKITKNLNSLSELEVKNLQRLSGAMDELHNEILELDEKVDD | | |
| | LRADTISSQIELAVLLSNEGIINSEDEHLLALERKLKKMLGPS | | |
| | AVEIGNGCFETKHKCNQTCLDKIAAGTFDAGEFSLPTFDSL | | |
| | NITAASLNDDGLDNHTILLYYSTAASSLAVTLMIAIFVVYM | | |
| | VSRDNVSCSICLVPRGSHHHHHH | | |
| 16 | ATGAAGGCCATCATCGTGCTTCTCATGGTGGTGACCAGC | | B/CO′17 HA0-FL- |
| | TCAGCGGACCGGATCTGCACCGGCATTACCAGCTCCAAC | | Thrombin-6xHis |
| | TCCCCCCACGTCGTGAAAACTGCGACCCAGGGAGAAGTG | | |
| | AACGTCACTGGCGTGATTCCGCTGACCACCACCCCCACC | | |
| | AAGTCCCATTTCGCCAACCTGAAGGGGACCGAAACACG | | |
| | GGGCAAACTCTGCCCGAAGTGCCTGAACTGTACCGATCT | | |
| | GGACGTGGCACTGGGAAGGCCAAAGTGCACCGGGAAGA | | |
| | TTCCGAGCGCCAGAGTGTCGATCTTGCACGAAGTCAGAC | | |
| | CTGTGACCTCGGGATGTTTCCCCATTATGCACGACCGGA | | |
| | CAAAGATCCGCCAGCTCCCTAATCTGTTGCGGGGATATG | | |
| | AGCACGTCCGCCTTTCGACTCACAACGTGATCAACGCCG | | |
| | AAGGCGCACCTGGTGGTCCTTACAAGATCGGGACTTCGG | | |
| | GTTCCTGCCCGAACATCACCAACGGAAACGGCTTTTTCG | | |
| | CCACCATGGCCTGGGCTGTGCCAGACAAGAACAAGACT | | |
| | GCCACCAATCCCCTGACCATCGAAGTGCCGTACGTGTGC | | |
| | ACGGAGGGGGAAGATCAGATTACTGTGTGGGGGTTCCA | | |
| | CAGCGATAACGAAACCCAGATGGCCAAGCTGTACGGAG | | |
| | ATTCAAAGCCCCAGAAATTCACTTCGAGCGCTAACGGTG | | |
| | TCACCACTCACTACGTGTCCCAAATCGGAGGGTTCCCGA | | |
| | ATCAAACCGAGGACGGGGGATTGCCGCAATCCGGTCGC | | |
| | ATCGTGGTCGACTATATGGTGCAGAAGTCGGGCAAAACT | | |
| | GGCACTATCACGTACCAGAGGGGAATCCTGCTGCCTCAA | | |
| | AAAGTGTGGTGTGCGTCAGGCCGGTCTAAGGTCATCAAG | | |
| | GGTTCCCTGCCCCTCATCGGAGAGGCCGACTGCCTCCAC | | |
| | GAAAAATACGGAGGCCTCAACAAGTCCAAGCCCTACTA | | |
| | CACCGGGGAACATGCCAAGGCCATCGGGAACTGCCCCA | | |
| | TTTGGGTTAAGACCCCACTGAAGCTCGCCAACGGCACTA | | |
| | AGTACAGACCTCCGGCCAAGTTGCTGAAGGAACGGGGA | | |
| | TTTTTCGGAGCCATTGCGGGATTCCTGGAAGGAGGCTGG | | |
| | GAGGGAATGATTGCGGGGTGGCACGGATACACTAGCCA | | |
| | TGGCGCTCACGGAGTGGCAGTGGCGGCAGACCTGAAGT | | |
| | CCACTCAGGAGGCCATCAACAAGATTACCAAGAACCTG | | |
| | AACAGCCTGTCCGAGCTGGAAGTCAAGAATCTCCAGAG | | |
| | GCTCAGCGGCGCTATGGACGAGCTTCATAATGAGATCCT | | |
| | GGAGCTGGATGAGAAGGTCGACGATCTCCGCGCGGACA | | |
| | CCATAAGCTCGCAGATCGAGCTGGCCGTGCTTCTGTCGA | | |
| | ACGAGGGCATCATCAACTCCGAGGACGAGCACCTCCTGG | | |
| | CACTTGAACGGAAGCTCAAGAAAATGCTGGGACCTTCCG | | |
| | CTGTGGAAATTGGCAACGGCTGCTTCGAGACTAAGCACA | | |
| | AGTGCAACCAGACGTGCCTGGATAAGATTGCCGCCGGA | | |
| | ACCTTCGACGCCGGAGAGTTTAGCCTGCCCACCTTCGAC | | |
| | TCCCTGAACATCACCGCGGCCTCACTGAATGATGACGGC | | |
| | CTTGATAACCACACCATCCTCCTGTACTACTCCACCGCCG | | |
| | CATCCTCACTCGCCGTGACTCTGATGATCGCCATCTTCGT | | |
| | GGTGTACATGGTCAGCCGCGACAACGTGTCCTGTTCCAT | | |
| | TTGCCTGGTGCCGAGAGGTTCCCACCATCATCACCATCA | | |
| | CTAATGA | | |
| 17 | YVADAPK | | Synthetic quencher |
| | | | peptide sequence |
| 18 | 1 | msssswllls lvavtaaqst ieeqaktfld kfnheaedlf yqsslaswny ntniteenvq | ACE-2 fragment |
| | 61 | nmnnagdkws aflkeqstla | |
| 19 | 1 | msssswllls lvavtaaqst ieeqaktfld kfnheaedlf yqsslaswny ntniteenvq | Human ACE-2 |
| | 61 | nmnnagdkws aflkeqstla qmyplqeiqn ltvklqlqal qqngssvlse dkskrlntil | protein |
| | 121 | ntmstiystg kvcnpdnpqe clllepglne imansldyne rlwaweswrs evgkqlrply | |
| | 181 | eeyvvlknem aranhyedyg dywrgdyevn gvdgydysrg qliedvehtf eeikplyehl | |
| | 241 | hayvraklmn aypsyispig clpahllgdm wgrfwtnlys ltvpfgqkpn idvtdamvdq | |
| | 301 | awdaqrifke aekffvsvgl pnmtqgfwen smltdpgnvq kavchptawd lgkgdfrilm | |
| | 361 | ctkvtmddfl tahhemghiq ydmayaaqpf llrnganegf heavgeimsl saatpkhlks | |
| | 421 | igllspdfqe dneteinfll kqaltivgtl pftymlekwr wmvfkgeipk dqwmkkwwem | |
| | 481 | kreivgvvep vphdetycdp aslfhvsndy sfiryytrtl yqfqfqealc qaakhegplh | |
| | 541 | kcdisnstea gqklfnmlrl gksepwtlal envvgaknmn vrpllnyfep lftwlkdqnk | |
| | 601 | nsfvgwstdw spyadqsikv rislksalgd kayewndnem ylfrssvaya mrqyflkvkn | |
| | 661 | qmilfgeedv rvanlkpris fnffvtapkn vsdiiprtev ekairmsrsr indafrlndn | |
| | 721 | sleflgiqpt igppnqppvs iwlivfgvvm gvivvgivil iftgirdrkk knkarsgenp | |
| | 781 | yasidiskge nnpgfqntdd vqtsf | |
| 20 | 1 | mgyinvfafp ftiyslllcr mnsrnyiaqv dvvnfnlt | ORF10 protein |
| | | | [Severe acute |
| | | | respiratory |
| | | | syndrome |
| | | | coronavirus 2]; |
| | | | NCBI Reference |
| | | | Sequence: |
| | | | YP_009725255.1 |
| 21 | 1 | msdngpqnqr napritfggp sdstgsnqng ersgarskqr rpqglpnnta swftaltqhg | nucleocapsid |
| | 61 | kedlkfprgq gvpintnssp ddqigyyrra trrirggdgk mkdlsprwyf yylgtgpeag | phosphoprotein |
| | 121 | lpygankdgi iwvategaln tpkdhigtrn pannaaivlq lpqgttlpkg fyaegsrggs | [Severe acute |
| | 181 | qassrsssrs rnssrnstpg ssrgtsparm agnggdaala lllldrlnql eskmsgkgqq | respiratory |
| | 241 | qqgqtvtkks aaeaskkprq krtatkaynv tqafgrrgpe qtqgnfgdqe lirqgtdykh | syndrome |
| | 301 | wpqiaqfaps asaffgmsri gmevtpsgtw ltytgaikld dkdpnfkdqv illnkhiday | coronavirus 2]; |
| | 361 | ktfpptepkk dkkkkadetq alpqrqkkqq tvtllpaadl ddfskqlqqs mssadstqa | NCBI Reference |
| | | | Sequence: |
| | | | YP_009724397.2 |
| 22 | 1 | mfhlvdfqvt iaeilliimr tfkvsiwnld yiinliiknl sksltenkys qldeeqpmei | ORF6 protein |
| | 61 | d | [Severe acute |
| | | | respiratory |
| | | | syndrome |
| | | | coronavirus 2]; |
| | | | NCBI Reference |
| | | | Sequence: |
| | | | YP_009724394.1 |
| 23 | 1 | meslvpgfne kthvqlslpv lqvrdvlvrg fgdsveevls earqhlkdgt cglvevekgv | orf1 ab polyprotein |
| | 61 | lpqleqpyvf ikrsdartap hghvmvelva elegiqygrs getlgvlvph vgeipvayrk | [Severe acute |
| | 121 | vllrkngnkg agghsygadl ksfdlgdelg tdpyedfqen wntkhssgvt relmrelngg | respiratory |
| | 181 | aytryvdnnf cgpdgyplec ikdllaragk asctlseqld fidtkrgvyc creheheiaw | syndrome |
| | 241 | yterseksye lqtpfeikla kkfdtfngec pnfvfplnsi iktiqprvek kkldgfmgri | coronavirus 2]; |
| | 301 | rsvypvaspn ecnqmclstl mkcdhcgets wqtgdfvkat cefcgtenlt kegattcgyl | NCBI Reference |
| | 361 | pqnavvkiyc pachnsevgp ehslaeyhne sglktilrkg grtiafggcv fsyvgchnkc | Sequence: |
| | 421 | aywvprasan igcnhtgvvg egseglndnl leilqkekvn inivgdfkln eeiaiilasf | YP_009724389.1 |
| | 481 | sastsafvet vkgldykafk qivescgnfk vtkgkakkga wnigeqksil splyafasea | |
| | 541 | arvvrsifsr tletaqnsvr vlqkaaitil dgisqyslrl idammftsdl atnnlvvmay | |
| | 601 | itggvvqlts qwltnifgtv yeklkpvldw leekfkegve flrdgweivk fistcaceiv | |
| | 661 | ggqivtcake ikesvqtffk lvnkflalca dsiiiggakl kalnlgetfv thskglyrkc | |
| | 721 | vksreetgll mplkapkeii flegetlpte vlteevvlkt gdlqpleqpt seaveaplvg | |
| | 781 | tpvcinglml leikdtekyc alapnmmvtn ntftlkggap tkvtfgddtv ievqgyksvn | |
| | 841 | itfelderid kvinekcsay tvelgtevne facvvadavi ktlqpvsell tplgidldew | |
| | 901 | smatyylfde sgefklashm ycsfyppded eeegdceeee fepstqyeyg teddyqgkpl | |
| | 961 | efgatsaalq peeeqeedwl dddsqqtvgq qdgsednqtt tiqtivevqp qlemeltpvv | |
| | 1021 | qtievnsfsg ylkltdnvyi knadiveeak kvkptvvvna anvylkhggg vagalnkatn | |
| | 1081 | namqvesddy iatngplkvg gscvlsghnl akhclhvvgp nvnkgediql lksayenfnq | |
| | 1141 | hevllaplls agifgadpih slrvcvdtvr tnvylavfdk nlydklvssf lemksekqve | |
| | 1201 | qkiaeipkee vkpfiteskp sveqrkqddk kikacveevt ttleetkflt enlllyidin | |
| | 1261 | gnlhpdsatl vsdiditflk kdapyivgdv vqegvltavv iptkkaggtt emlakalrkv | |
| | 1321 | ptdnyittyp gqglngytve eaktvlkkck safyilpsii snekqeilgt vswnlremla | |
| | 1381 | haeetrklmp vcvetkaivs tiqrkykgik iqegvvdyga rfyfytsktt vaslintlnd | |
| | 1441 | lnetlvtmpl gyvthglnle eaarymrslk vpatvsvssp davtayngyl tsssktpeeh | |
| | 1501 | fietislags ykdwsysgqs tqlgieflkr gdksvyytsn pttfhldgev itfdnlktll | |
| | 1561 | slrevrtikv fttvdninlh tqvvdmsmty gqqfgptyld gadvtkikph nshegktfyv | |
| | 1621 | lpnddtlrve afeyyhttdp sflgrymsal nhtkkwkypq vngltsikwa dnncylatal | |
| | 1681 | ltlqqielkf nppalqdayy rarageaanf calilaycnk tvgelgdvre tmsylfqhan | |
| | 1741 | ldsckrvlnv vcktcgqqqt tlkgveavmy mgtlsyeqfk kgvqipctcg kqatkylvqq | |
| | 1801 | espfvmmsap paqyelkhgt ftcaseytgn yqcghykhit sketlycidg alltksseyk | |
| | 1861 | gpitdvfyke nsytttikpv tykldgvvct eidpkldnyy kkdnsyfteq pidlvpnqpy | |
| | 1921 | pnasfdnfkf vcdnikfadd lnqltgykkp asrelkvtff pdlngdvvai dykhytpsfk | |
| | 1981 | kgakllhkpi vwhvnnatnk atykpntwci rclwstkpve tsnsfdvlks edaqgmdnla | |
| | 2041 | cedlkpvsee vvenptiqkd vlecnvktte vvgdiilkpa nnslkiteev ghtdlmaayv | |
| | 2101 | dnssltikkp nelsrvlglk tlathglaav nsvpwdtian yakpflnkvv stttnivtrc | |
| | 2161 | lnrvctnymp yfftlllqlc tftrstnsri kasmpttiak ntvksvgkfc leasfnylks | |
| | 2221 | pnfsklinii iwflllsvcl gsliystaal gvlmsnlgmp syetgyregy lnstnvtiat | |
| | 2281 | yctgsipcsv clsgldsldt ypsletiqit issfkwdlta fglvaewfla yilftrffyv | |
| | 2341 | lglaaimqlf fsyfavhfis nswlmwliin lvqmapisam vrmyiffasf yyvwksyvhv | |
| | 2401 | vdgcnsstcm mcykrnratr vecttivngv rrsfyvyang gkgfcklhnw ncvncdtfca | |
| | 2461 | gstfisdeva rdlslqfkrp inptdqssyi vdsvtvkngs ihlyfdkagq ktyerhslsh | |
| | 2521 | fvnldnlran ntkgslpinv ivfdgkskce essaksasvy ysqlmcqpil lldqalvsdv | |
| | 2581 | gdsaevavkm fdayvntfss tfnvpmeklk tlvataeael aknvsldnvl stfisaarqg | |
| | 2641 | fvdsdvetkd vveclklshq sdievtgdsc nnymltynkv enmtprdlga cidcsarhin | |
| | 2701 | aqvakshnia liwnvkdfms lseqlrkqir saakknnlpf kltcattrqv vnvvttkial | |
| | 2761 | kggkivnnwl kqlikvtlvf lfvaaifyli tpvhvmskht dfsseiigyk aidggvtrdi | |
| | 2821 | astdtcfank hadfdtwfsq rggsytndka cpliaavitr evgfvvpglp gtilrttngd | |
| | 2881 | flhflprvfs avgnicytps klieytdfat sacvlaaect ifkdasgkpv pycydtnvle | |
| | 2941 | gsvayeslrp dtryvlmdgs iiqfpntyle gsvrvvttfd seycrhgtce rseagvcvst | |
| | 3001 | sgrwvlnndy yrslpgvfcg vdavnlltnm ftpliqpiga ldisasivag givaivvtcl | |
| | 3061 | ayyfmrfrra fgeyshvvaf ntllflmsft vlcltpvysf lpgvysviyl yltfyltndv | |
| | 3121 | sflahiqwmv mftplvpfwi tiayiicist khfywffsny lkrrvvfngv sfstfeeaal | |
| | 3181 | ctfllnkemy lklrsdvllp ltqynrylal ynkykyfsga mdttsyreaa cchlakalnd | |
| | 3241 | fsnsgsdvly qppqtsitsa vlqsgfrkma fpsgkvegcm vqvtcgtttl nglwlddvvy | |
| | 3301 | cprhvictse dmlnpnyedl lirksnhnfl vqagnvqlrv ighsmqncvl klkvdtanpk | |
| | 3361 | tpkykfvriq pgqtfsvlac yngspsgvyq camrpnftik gsflngscgs vgfnidydcv | |
| | 3421 | sfcymhhmel ptgvhagtdl egnfygpfvd rqtaqaagtd ttitvnvlaw lyaavingdr | |
| | 3481 | wflnrftttl ndfnlvamky nyepltqdhv dilgplsaqt giavldmcas lkellqngmn | |
| | 3541 | grtilgsall edeftpfdvv rqcsgvtfqs avkrtikgth hwllltilts llvlvqstqw | |
| | 3601 | slffflyena flpfamgiia msafammfvk hkhaflclfl lpslatvayf nmvympaswv | |
| | 3661 | mrimtwldmv dtslsgfklk dcvmyasavv llilmtartv yddgarrvwt lmnvltlvyk | |
| | 3721 | vyygnaldqa ismwaliisv tsnysgvvtt vmflargivf mcveycpiff itgntlqcim | |
| | 3781 | lvycflgyfc tcyfglfcll nryfrltlgv ydylvstqef rymnsqgllp pknsidafkl | |
| | 3841 | nikllgvggk pcikvatvqs kmsdvkctsv vllsvlqqlr vesssklwaq cvqlhndill | |
| | 3901 | akdtteafek mvsllsvlls mqgavdinkl ceemldnrat lqaiasefss lpsyaafata | |
| | 3961 | qeayeqavan gdsevvlkkl kkslnvakse fdrdaamqrk lekmadqamt qmykqarsed | |
| | 4021 | krakvtsamq tmlftmlrkl dndalnniin nardgcvpln iiplttaakl mvvipdynty | |
| | 4081 | kntcdgttft yasalweiqq vvdadskivq lseismdnsp nlawplivta lransavklq | |
| | 4141 | nnelspvalr qmscaagttq tactddnala yynttkggrf vlallsdlqd lkwarfpksd | |
| | 4201 | gtgtiytele ppcrfvtdtp kgpkvkylyf ikglnnlnrg mvlgslaatv rlqagnatev | |
| | 4261 | panstvlsfc afavdaakay kdylasggqp itncvkmlct htgtgqaitv tpeanmdqes | |
| | 4321 | fggascclyc rchidhpnpk gfcdlkgkyv qipttcandp vgftlkntvc tvcgmwkgyg | |
| | 4381 | cscdqlrepm lqsadaqsfl nrvcgvsaar ltpcgtgtst dvvyrafdiy ndkvagfakf | |
| | 4441 | lktnccrfqe kdeddnlids yfvvkrhtfs nyqheetiyn llkdcpavak hdffkfridg | |
| | 4501 | dmvphisrqr ltkytmadlv yalrhfdegn cdtlkeilvt ynccdddyfh kkdwydfven | |
| | 4561 | pdilrvyanl gervrqallk tvqfcdamrn agivgvltld nqdlngnwyd fgdfiqttpg | |
| | 4621 | sgvpvvdsyy sllmpiltlt raltaeshvd tdltkpyikw dllkydftee rlklfdryfk | |
| | 4681 | ywdqtyhpnc vnclddrcil hcanfnvlfs tvfpptsfgp lvrkifvdgv pfvvstgyhf | |
| | 4741 | relgvvhnqd vnlhssrlsf kellvyaadp amhaasgnll ldkrttcfsv aaltnnvafq | |
| | 4801 | tvkpgnfnkd fydfavskgf fkegssvelk hfffaqdgna aisdydyyry nlptmcdirq | |
| | 4861 | llfvvevvdk yfdcydggci nanqvivnnl dksagfpfnk wgkarlyyds msyedqdalf | |
| | 4921 | aytkrnvipt itqmnlkyai saknrartva gvsicstmtn rqfhqkllks iaatrgatvv | |
| | 4981 | igtskfyggw hnmlktvysd venphlmgwd ypkcdrampn mlrimaslvl arkhttccsl | |
| | 5041 | shrfyrlane caqvlsemvm cggslyvkpg gtssgdatta yansvfnicq avtanvnall | |
| | 5101 | stdgnkiadk yvrnlqhrly eclyrnrdvd tdfvnefyay lrkhfsmmil sddavvcfns | |
| | 5161 | tyasqglvas iknfksvlyy qnnvfmseak cwtetdltkg phefcsqhtm lvkqgddyvy | |
| | 5221 | lpypdpsril gagcfvddiv ktdgtlmier fvslaidayp ltkhpnqeya dvfhlylqyi | |
| | 5281 | rklhdeltgh mldmysvmlt ndntsrywep efyeamytph tvlqavgacv lcnsqtslrc | |
| | 5341 | gacirrpflc ckccydhvis tshklvlsvn pyvcnapgcd vtdvtqlylg gmsyyckshk | |
| | 5401 | ppisfplcan gqvfglyknt cvgsdnvtdf naiatcdwtn agdyilantc terlklfaae | |
| | 5461 | tlkateetfk lsygiatvre vlsdrelhls wevgkprppl nrnyvftgyr vtknskvqig | |
| | 5521 | eytfekgdyg davvyrgttt yklnvgdyfv ltshtvmpls aptlvpqehy vritglyptl | |
| | 5581 | nisdefssnv anyqkvgmqk ystlqgppgt gkshfaigla lyypsarivy tacshaavda | |
| | 5641 | lcekalkylp idkcsriipa rarvecfdkf kvnstleqyv fctvnalpet tadivvfdei | |
| | 5701 | smatnydlsv vnarlrakhy vyigdpaqlp aprtlltkgt lepeyfnsvc rlmktigpdm | |
| | 5761 | flgtcrrcpa eivdtvsalv ydnklkahkd ksaqcfkmfy kgvithdvss ainrpqigvv | |
| | 5821 | refltrnpaw rkavfispyn sqnavaskil glptqtvdss qgseydyvif tqttetahsc | |
| | 5881 | nvnrfnvait rakvgilcim sdrdlydklq ftsleiprrn vatlqaenvt glfkdcskvi | |
| | 5941 | tglhptqapt hlsvdtkfkt eglcvdipgi pkdmtyrrli smmgfkmnyq vngypnmfit | |
| | 6001 | reeairhvra wigfdvegch atreavgtnl plqlgfstgv nlvavptgyv dtpnntdfsr | |
| | 6061 | vsakpppgdq fkhliplmyk glpwnvvrik ivqmlsdtlk nlsdrvvfvl wahgfeltsm | |
| | 6121 | kyfvkigper tccledrrat cfstasdtya cwhhsigfdy vynpfmidvq qwgftgnlqs | |
| | 6181 | nhdlycqvhg nahvascdai mtrclavhec fvkrvdwtie ypiigdelki naacrkvqhm | |
| | 6241 | vvkaalladk fpvlhdignp kaikcvpqad vewkfydaqp csdkaykiee lfysyathsd | |
| | 6301 | kftdgvclfw ncnvdrypan sivcrfdtrv lsnlnlpgcd ggslyvnkha fhtpafdksa | |
| | 6361 | fvnlkqlpff yysdspcesh gkqvvsdidy vplksatcit rcnlggavcr hhaneyrlyl | |
| | 6421 | daynmmisag fslwvykqfd tynlwntftr lqslenvafn vvnkghfdgq qgevpvsiin | |
| | 6481 | ntvytkvdgv dvelfenktt lpvnvafelw akrnikpvpe vkilnnlgvd iaantviwdy | |
| | 6541 | krdapahist igvcsmtdia kkpteticap ltvffdgrvd gqvdlfrnar ngvlitegsv | |
| | 6601 | kglqpsvgpk qaslngvtli geavktqfny ykkvdgvvqq lpetyftqsr nlqefkprsq | |
| | 6661 | meidflelam defierykle gyafehivyg dfshsqlggl hlliglakrf kespfeledf | |
| | 6721 | ipmdstvkny fitdaqtgss kcvcsvidll lddfveiiks qdlsvvskvv kvtidyteis | |
| | 6781 | fmlwckdghv etfypklqss qawqpgvamp nlykmqrmll ekcdlqnygd satlpkgimm | |
| | 6841 | nvakytqlcq ylntltlavp ynmrvihfga gsdkgvapgt avlrqwlptg tllvdsdlnd | |
| | 6901 | fvsdadstli gdcatvhtan kwdliisdmy dpktknvtke ndskegffty icgfiqqkla | |
| | 6961 | lggsvaikit ehswnadlyk lmghfawwta fvtnvnasss eafligcnyl gkpreqidgy | |
| | 7021 | vmhanyifwr ntnpiqlssy slfdmskfpl klrgtavmsl kegqindmil sllskgrlii | |
| | 7081 | rennrvviss dvlvnn | |
| 24 | 1 | madsngtitv eelkklleqw nlvigflflt wicllqfaya nrnrflyiik liflwllwpv | membrane |
| | 61 | tlacfvlaav yrinwitggi aiamaclvgl mwlsyfiasf rlfartrsmw sfnpetnill | glycoprotein |
| | 121 | nvplhgtilt rplleselvi gavilrghlr iaghhlgrcd ikdlpkeitv atsrtlsyyk | [Severe acute |
| | 181 | lgasqrvagd sgfaaysryr ignyklntdh ssssdniall vq | respiratory |
| | | | syndrome |
| | | | coronavirus 2]; |
| | | | NCBI Reference |
| | | | Sequence: |
| | | | YP_009724393.1 |
| 25 | 1 | mfvflvllpl vssqcvnltt rtqlppaytn sftrgvyypd kvfrssvlhs tqdlflpffs | surface |
| | 61 | nvtwfhaihv sgtngtkrfd npvlpfndgv yfasteksni irgwifgttl dsktqslliv | glycoprotein |
| | 121 | nnatnvvikv cefqfendpf lgvyyhknnk swmesefrvy ssannctfey vsqpflmdle | [Severe acute |
| | 181 | gkqgnfknlr efvfknidgy fkiyskhtpi nlvrdlpqgf saleplvdlp iginitrfqt | respiratory |
| | 241 | llalhrsylt pgdsssgwta gaaayyvgyl qprtfllkyn engtitdavd caldplsetk | syndrome |
| | 301 | ctlksftvek giyqtsnfrv qptesivrfp nitnlcpfge vfnatrfasv yawnrkrisn | coronavirus 2]; |
| | 361 | cvadysvlyn sasfstfkcy gvsptklndl cftnvyadsf virgdevrqi apgqtgkiad | NCBI Reference |
| | 421 | ynyklpddft gcviawnsnn ldskvggnyn ylyrlfrksn lkpferdist eiyqagstpc | Sequence: |
| | 481 | ngvegfncyf plqsygfqpt ngvgyqpyrv vvlsfellha patvcgpkks tnlvknkcvn | YP_009724390.1 |
| | 541 | fnfngltgtg vltesnkkfl pfqqfgrdia dttdavrdpq tleilditpc sfggvsvitp | |
| | 601 | gtntsnqvav lyqdvnctev pvaihadqlt ptwrvystgs nvfqtragcl igaehvnnsy | |
| | 661 | ecdipigagi casyqtqtns prrarsvasq siiaytmslg aensvaysnn siaiptnfti | |
| | 721 | svtteilpvs mtktsvdctm yicgdstecs nlllqygsfc tqlnraltgi aveqdkntqe | |
| | 781 | vfaqvkqiyk tppikdfggf nfsqilpdps kpskrsfied llfnkvtlad agfikqygdc | |
| | 841 | lgdiaardli caqkfngltv lpplltdemi aqytsallag titsgwtfga gaalqipfam | |
| | 901 | qmayrfngig vtqnvlyenq klianqfnsa igkiqdslss tasalgklqd vvnqnaqaln | |
| | 961 | tlvkqlssnf gaissvlndi lsrldkveae vqidrlitgr lqslqtyvtq qliraaeira | |
| | 1021 | sanlaatkms ecvlgqskrv dfcgkgyhlm sfpqsaphgv vflhvtyvpa qeknfttapa | |
| | 1081 | ichdgkahfp regvfvsngt hwfvtqrnfy epqiittdnt fvsgncdvvi givnntvydp | |
| | 1141 | lqpeldsfke eldkyfknht spdvdlgdis ginasvvniq keidrlneva knlneslidl | |
| | 1201 | qelgkyeqyi kwpwyiwlgf iagliaivmv timlccmtsc csclkgccsc gscckfdedd | |
| | 1261 | sepvlkgvkl hyt | |
| 26 | 1 | mdlfmrifti gtvtlkqgei kdatpsdfvr atatipiqas lpfgwlivgv allavfqsas | ORF3a protein |
| | 61 | kiitlkkrwq lalskgvhfv cnllllfvtv yshlllvaag leapflylya lvyflqsinf | [Severe acute |
| | 121 | vriimrlwlc wkcrsknpll ydanyflcwh tncydycipy nsvtssivit sgdgttspis | respiratory |
| | 181 | ehdyqiggyt ekwesgvkdc vvlhsyftsd yyqlystqls tdtgvehvtf fiynkivdep | syndrome |
| | 241 | eehvqihtid gssgvvnpvm epiydepttt tsvpl | coronavirus 2]; |
| | | | NCBI Reference |
| | | | Sequence: |
| | | | YP_009724391.1 |
| 27 | 1 | mkiilflali tlatcelyhy qecvrgttvl lkepcssgty egnspfhpla dnkfaltcfs | ORF7a protein |
| | 61 | tqfafacpdg vkhvyqlrar svspklfirq eevqelyspi flivaaivfi tlcftlkrkt | [Severe acute |
| | 121 | e | respiratory |
| | | | syndrome |
| | | | coronavirus 2]; |
| | | | NCBI Reference |
| | | | Sequence: |
| | | | YP_009724395.1 |
| 28 | 1 | mkflvflgii ttvaafhqec slqsctqhqp yvvddpcpih fyskwyirvg arksapliel | ORF8 protein |
| | 61 | cvdeagsksp iqyidignyt vsclpftinc qepklgslvv resfyedfle yhdvrvvldf | [Severe acute |
| | 121 | i | respiratory |
| | | | syndrome |
| | | | coronavirus 2]; |
| | | | NCBI Reference |
| | | | Sequence: |
| | | | YP_009724396.1 |
| 29 | 1 | mysfvseetg tlivnsvllf lafvvfllvt lailtalrlc ayccnivnvs lvkpsfyvys | envelope protein |
| | 61 | rvknlnssrv pdllv | [Severe acute |
| | | | respiratory |
| | | | syndrome |
| | | | coronavirus 2]; |
| | | | NCBI Reference |
| | | | Sequence: |
| | | | YP_009724392.1 |

| | | |
|---|---|---|
| 30 | MFVFLVLLPLVSSQCVNLTTRTQLPPAYTNSFTRGVYYPDK | Amino acid |
| | VFRSSVLHSTQDLFLPFFSNVTWFHAIHVSGTNGTKRFDNP | sequence of a |
| | VLPFNDGVYFASTEKSNIIRGWIFGTTLDSKTQSLLIVNNAT | synthetic construct |
| | NVVIKVCEFQFCNDPFLGVYYHKNNKSWMESEFRVYSSAN | |
| | NCTFEYVSQPFLMDLEGKQGNFKNLREFVFKNIDGYFKIYS | |
| | KHTPINLVRDLPQGFSALEPLVDLPIGINITRFQTLLALHRSY | |
| | LTPGDSSSGWTAGAAAYYVGYLQPRTFLLKYNENGTITDA | |
| | VDCALDPLSETKCTLKSFTVEKGIYQTSNFRVQPTESIVRFP | |
| | NITNLCPFGEVFNATRFASVYAWNRKRISNCVADYSVLYNS | |
| | ASFSTFKCYGVSPTKLNDLCFTNVYADSFVIRGDEVRQIAP | |
| | GQTGKIADYNYKLPDDFTGCVIAWNSNNLDSKVGGNYNY | |
| | LYRLFRKSNLKPFERDISTEIYQAGSTPCNGVEGFNCYFPLQ | |
| | SYGFQPTNGVGYQPYRVVVLSFELLHAPATVCGPKKSTNL | |
| | VKNKCVNFNFNGLTGTGVLTESNKKFLPFQQFGRDIADTT | |
| | DAVRDPQTLEILDITPCSFGGVSVITPGTNTSNQVAVLYQDV | |
| | NCTEVPVAIHADQLTPTWRVYSTGSNVFQTRAGCLIGAEH | |
| | VNNSYECDIPIGAGICASYQTQTNSPRRARSVASQSIIAYTM | |
| | SLGAENSVAYSNNSIAIPTNFTISVTTEILPVSMTKTSVDCTM | |
| | YICGDSTECSNLLLQYGSFCTQLNRALTGIAVEQDKNTQEV | |
| | FAQVKQIYKTPPIKDFGGFNFSQILPDPSKPSKRSFIEDLLFN | |
| | KVTLADAGFIKQYGDCLGDIAARDLICAQKFNGLTVLPPLL | |
| | TDEMIAQYTSALLAGTITSGWTFGAGAALQIPFAMQMAYR | |
| | FNGIGVTQNVLYENQKLIANQFNSAIGKIQDSLSSTASALGK | |
| | LQDVVNQNAQALNTLVKQLSSNFGAISSVLNDILSRLDKVE | |
| | AEVQIDRLITGRLQSLQTYVTQQLIRAAEIRASANLAATKM | |
| | SECVLGQSKRVDFCGKGYHLMSFPQSAPHGVVFLHVTYVP | |
| | AQEKNFTTAPAICHDGKAHFPREGVFVSNGTHWFVTQRNF | |
| | YEPQIITTDNTFVSGNCDVVIGIVNNTVYDPLQPELDSFKEE | |
| | LDKYFKNHTSPDVDLGDISGINASVVNIQKEIDRLNEVAKN | |
| | LNESLIDLQELGKYEQYIKWPWYIWLGFIAGLIAIVMVTIML | |
| | CCMTSCCSCLKGCCSCGSCCKFDEDDSEPVLKGVKLHYTL | |
| | VPRGSHHHHHH | |
| 31 | ATGTTTGTGTTCCTCGTGCTGCTCCCTCTCGTGTCCTCCC | VrS01 ORF DNA |
| | AATGCGTGAATCTGACCACCCGGACTCAGCTGCCCCCGG | sequence |
| | CTTACACAAACAGCTTCACCCGGGGCGTTTACTACCCGG | |
| | ACAAAGTGTTCCGGTCAAGCGTGCTGCATAGCACCCAGG | |
| | ATCTGTTCCTGCCGTTCTTCTCGAACGTGACCTGGTTCCA | |
| | CGCCATCCACGTGTCCGGAACCAACGGGACCAAGAGATT | |
| | CGACAACCCTGTCCTGCCGTTTAACGACGGAGTGTACTT | |
| | CGCGTCCACCGAAAAGTCGAACATCATCCGCGGCTGGAT | |
| | TTTCGGGACTACCCTGGACTCCAAGACTCAATCCCTCCTC | |
| | ATCGTCAACAACGCCACCAATGTCGTGATCAAGGTCTGC | |
| | GAGTTTCAGTTCTGCAACGATCCCTTTCTCGGCGTGTACT | |
| | ACCACAAGAACAACAAGTCGTGGATGGAGTCCGAGTTTC | |
| | GCGTGTACTCCTCCGCCAACAACTGCACCTTCGAATACG | |
| | TGTCCCAGCCATTCCTGATGGACCTGGAGGGAAAGCAGG | |
| | GAAACTTCAAGAACCTGAGAGAGTTCGTGTTTAAGAATA | |
| | TTGACGGATACTTCAAGATATACTCCAAGCACACTCCGA | |
| | TCAACTTGGTCCGGGATCTGCCGCAAGGATTCTCAGCGC | |
| | TGGAACCACTGGTCGACCTTCCCATCGGCATCAACATTA | |
| | CACGGTTCCAGACCTTGCTGGCCCTGCATAGAAGCTACC | |
| | TTACCCCCGGGGACTCCTCCTCCGGATGGACCGCCGGCG | |
| | CAGCAGCCTACTACGTGGGATACCTCCAGCCCCGCACTT | |
| | TCCTGCTGAAGTACAACGAAAACGGAACCATCACCGAC | |
| | GCCGTGGACTGTGCTCTGGATCCCCTGTCCGAGACTAAG | |
| | TGTACCTTGAAGTCATTCACCGTGGAAAAGGGAATCTAT | |
| | CAGACCTCAAATTTTCGGGTGCAGCCCACCGAGTCCATC | |
| | GTGCGGTTTCCCAACATCACTAACCTCTGCCCGTTCGGG | |
| | GAAGTGTTTAACGCGACCAGATTCGCCAGCGTGTACGCA | |
| | TGGAATCGGAAGAGGATTAGCAACTGCGTGGCCGATTAC | |
| | TCCGTGCTCTACAACTCGGCCAGCTTTAGCACCTTCAAGT | |
| | GCTACGGAGTGTCCCCGACGAAGCTGAACGACCTGTGCT | |
| | TCACTAACGTGTACGCCGACTCCTTCGTGATCCGGGGAG | |
| | ATGAAGTCCGCCAGATCGCACCTGGACAGACTGGCAAA | |
| | ATCGCCGACTATAATTACAAGCTGCCTGATGACTTCACT | |
| | GGCTGCGTCATTGCGTGGAACAGCAACAACCTCGACTCC | |
| | AAAGTCGGCGGAAATTACAACTATCTGTACCGCCTGTTT | |
| | CGAAAGAGCAACTTGAAGCCATTCGAACGGGACATTAG | |
| | CACCGAGATCTACCAGGCTGGATCTACCCCATGCAACGG | |
| | AGTGGAAGGCTTTAACTGCTACTTCCCACTGCAATCATA | |
| | CGGATTCCAGCCGACCAACGGCGTGGGTTACCAGCCATA | |
| | TCGGGTCGTGGTGCTGTCCTTCGAATTGCTGCATGCCCCA | |
| | GCCACCGTCTGCGGACCCAAGAAGTCCACGAACCTAGTG | |
| | AAGAATAAGTGCGTGAACTTCAACTTCAACGGATTAACT | |
| | GGCACCGGGGTCCTTACCGAATCCAACAAGAAATTTCTG | |
| | CCTTTCCAACAATTCGGTCGGGACATCGCAGACACTACT | |
| | GACGCCGTCAGGGACCCGCAGACCCTCGAAATTCTGGAT | |
| | ATCACACCTTGCTCCTTCGGCGGGGTGTCGGTGATCACC | |
| | CCTGGAACCAACACCTCGAACCAAGTCGCTGTGCTGTAC | |
| | CAGGATGTGAACTGTACCGAAGTGCCCGTGGCCATCCAC | |
| | GCTGACCAGCTGACTCCAACTTGGAGAGTCTACAGCACC | |
| | GGCTCGAACGTGTTCCAGACCCGGGCTGGCTGCCTCATT | |
| | GGCGCGGAACACGTGAACAACTCCTACGAGTGTGACATC | |
| | CCGATTGGCGCTGGGATTTGTGCGTCGTACCAGACTCAG | |
| | ACGAACTCCCCCCGCCGGGCCCGGTCCGTGGCGTCACAG | |
| | TCCATCATCGCGTACACCATGTCGCTGGGCGCCGAGAAC | |
| | AGCGTGGCCTACTCCAACAACTCGATTGCAATCCCTACT | |
| | AACTTCACTATCTCCGTGACTACCGAGATTCTGCCCGTGT | |
| | CCATGACAAAGACTTCGGTGGACTGCACTATGTACATCT | |
| | GTGGGGATAGTACCGAGTGCTCCAATCTGCTGCTTCAGT | |
| | ACGGATCCTTCTGTACCCAACTCAACCGCGCACTCACCG | |
| | GTATTGCGGTAGAACAGGACAAGAACACCCAGGAAGTG | |
| | TTCGCCCAAGTCAAGCAGATCTACAAGACCCCGCCCATC | |
| | AAGGACTTCGGCGGATTCAACTTCTCCCAAATCCTGCCT | |
| | GACCCGTCAAAGCCCTCCAAGCGGTCATTCATCGAGGAT | |
| | CTGTTGTTCAACAAGGTCACCCTGGCCGACGCCGGCTTC | |
| | ATCAAGCAATACGGAGACTGTCTCGGTGATATCGCCGCC | |
| | CGCGATCTGATTTGCGCGCAGAAGTTCAACGGGCTGACC | |
| | GTGCTGCCCCCTCTTTTGACTGATGAAATGATCGCCCAGT | |
| | ACACCTCGGCGCTGTTGGCGGGAACCATTACCTCCGGTT | |
| | GGACCTTCGGCGCGGGCGCTGCACTCCAAATTCCGTTTG | |
| | CCATGCAAATGGCCTACCGCTTCAACGGAATCGGCGTGA | |
| | CCCAGAACGTGCTGTACGAGAACCAGAAGCTGATCGCG | |
| | AACCAGTTCAACTCAGCCATTGGCAAAATCCAGGACTCG | |
| | CTGTCGTCCACTGCATCCGCCCTCGGGAAGCTTCAAGAC | |
| | GTCGTCAACCAGAACGCCCAGGCCCTCAACACCCTTGTG | |
| | AAACAGCTGAGCTCCAACTTCGGAGCCATTTCATCGGTG | |
| | CTTAATGACATCCTGAGCCGCCTGGACAAAGTGGAAGCC | |
| | GAAGTGCAGATTGACCGGCTTATCACCGGTCGCCTGCAG | |
| | TCACTCCAGACTTATGTGACCCAGCAGCTGATCCGCGCC | |
| | GCCGAGATCAGGGCCAGCGCGAACCTCGCTGCCACTAA | |
| | GATGTCCGAATGCGTGTTGGGACAGTCCAAGAGAGTGG | |
| | ACTTCTGCGGGAAAGGCTACCACCTGATGTCCTTCCCGC | |
| | AATCCGCACCGCACGGAGTCGTGTTCCTGCACGTGACCT | |
| | ACGTGCCGGCCCAGGAAAAGAATTTCACTACTGCGCCTG | |
| | CCATCTGCCACGACGGGAAGGCTCATTTCCCGAGAGAGG | |
| | GAGTGTTCGTGTCCAACGGTACCCACTGGTTCGTGACTC | |
| | AACGGAACTTCTACGAACCTCAGATTATCACCACCGATA | |
| | ACACGTTCGTGTCGGGGAACTGTGACGTCGTGATTGGAA | |
| | TCGTGAACAACACGGTGTACGACCCGCTGCAGCCCGAGC | |
| | TTGATTCCTTCAAGGAGGAGCTGGACAAGTACTTCAAGA | |
| | ATCACACCTCCCCTGATGTGGACCTGGGAGACATCAGCG | |
| | GCATTAACGCCTCTGTGGTCAACATCCAAAAGGAGATTG | |
| | ACAGACTCAACGAGGTCGCCAAGAACCTCAACGAGTCC | |
| | CTGATCGATCTGCAAGAACTGGGAAAATACGAACAGTA | |
| | CATTAAGTGGCCGTGGTACATCTGGCTGGGCTTCATCGC | |
| | CGGACTGATCGCCATCGTCATGGTCACTATCATGCTCTG | |
| | CTGCATGACCAGCTGCTGCAGCTGTCTGAAGGGTTGCTG | |
| | CTCGTGCGGATCCTGCTGCAAGTTCGACGAAGATGACTC | |
| | CGAGCCCGTGCTGAAGGGTGTCAAGCTGCATTACACCTT | |
| | GGTGCCTAGGGGTTCGCACCATCACCACCATCACTAATG | |
| | A | |

WSGR Docket No. 47750-705.301

1. A virus-like particle (VLP), comprising:
(a) a synthetic, semisynthetic or natural lipid bilayer;
(b) an anchor molecule embedded in the lipid bilayer; and
(c) an antigen bound to the anchor molecule.

2. The VLP of claim 1, wherein the lipid bilayer comprises a first lipid and a second lipid, wherein
i) the first lipid comprises a phosphatidylcholine species;
ii) the second lipid comprises a phosphatidylethanolamine species;
iii) the first lipid and/or the second lipid each comprise an acyl chain comprising between 4 and 18 carbon atoms;
iv) the first lipid and/or the second lipid each comprise four or less unsaturated bonds;
v) the first lipid and/or the second lipid are synthetic;
vi) the lipid bilayer, the first lipid, and/or the second lipid are at least 99% pure, or are free or substantially free of biologic material;
vii) the first lipid comprises DOPC;
viii) the second lipid comprises DOPE;
ix) the lipid bilayer comprises the first lipid and the second lipid at a predetermined ratio between 1:0.25 and 1:4;
x) the lipid bilayer comprises a sterol or sterol derivative;
xi) the lipid bilayer comprises cholesterol or DC-cholesterol; and/or
xii) the lipid bilayer comprises a sterol or sterol derivative at a ratio of 0-30 mol % in relation to the first lipid and/or the second lipid.

3.-13. (canceled)

14. The VLP of claim 1, wherein the antigen
i) is at least 75.0%, 80.0%, 85.0%, 90.0%, 91.0%, 92.0%, 93.0%, 94.0%, 95.0%, 96.0%, 97.0%, 97.5%, 98.0%, 98.5%, 99.0%, 99.5%, 99.9%, 100%, or a range of percentages defined by any two of the aforementioned percentages, pure;
ii) is bound directly to the anchor molecule, or wherein the antigen comprises the anchor molecule;
iii) comprises a bacterial antigen, or a fragment thereof;
iv) comprises a bacterial antigen, or a fragment thereof, wherein the bacterial antigen comprises an Actinomyces antigen, Bacillus antigens, immunogenic antigens from Bacillus anthracis, Bacteroides antigens, Bordetella antigens, Bartonella antigens, Borrelia antigens, B. burgdorferi OspA, Brucella antigens, Campylobacter antigens, Capnocytophaga antigens, Chlamydia antigens, Clostridium antigens, Corynebacterium antigens, Coxiella antigens, Dermatophilus antigens, Enterococcus antigens, Ehrlichia antigens, Escherichia antigens, Francisella antigens, Fusobacterium antigens, Haemobartonella antigens, Haemophilus antigens, H. influenzae type b outer membrane protein, Helicobacter antigens, Klebsiella antigens, L form bacteria antigens, Leptospira antigens, Listeria antigens, Mycobacteria antigens, Mycoplasma antigens, Neisseria antigens, Neorickettsia antigens, Nocardia antigens, Pasteurella antigens, Peptococcus antigens, Peptostreptococcus antigens, Pneumococcus antigens, Proteus antigens, Pseudomonas antigens, Rickettsia antigens, Rochalimaea antigens, Salmonella antigens, Shigella antigens, Staphylococcus antigens, Streptococcus antigens, S. pyogenes M proteins, Treponema antigens, Yersinia antigens, or Y. pestis F1 or V antigens;
v) comprises a fungal antigen, or a fragment thereof;
vi) comprises a fungal antigen, or a fragment thereof, wherein the fungal antigen comprises a Balantidium coli antigens, Entamoeba histolytica antigens, Fasciola hepatica antigens, Giardia lamblia antigens, Leishmania antigens, or Plasmodium antigens;
vii) comprises a cancer antigen, or a fragment thereof;
viii) comprises a cancer antigen, or a fragment thereof, wherein the cancer antigen comprises tumor-specific immunoglobulin variable regions, GM2, Tn, sTn, Thompson-Friedenreich antigen (TF), Globo H, Le(y), MUC1, MUC2, MUC3, MUC4, MUCSAC, MUCSB, MUC7, carcinoembryonic antigens, beta chain of human chorionic gonadotropin (hCG beta), C35, HER2/neu, CD20, PSMA, EGFRvIII, KSA, PSA, PSCA, GP100, MAGE 1, MAGE 2, TRP 1, TRP 2, tyrosinase, MART-1, PAP, CEA, BAGE, MAGE, or RAGE;
ix) comprises a viral antigen, or a fragment thereof;
x) comprises a viral antigen, or a fragment thereof, wherein the viral antigen comprises an antigen from a human immunodeficiency virus, (HIV), a flu virus, a Dengue virus, a Zika virus, a West Nile virus, an Ebola virus, Marburg virus, Rabies virus, a coronavirus, a Middle Eastern respiratory syndrome (MERS) virus, a severe acute respiratory syndrome (SARS) virus, a respiratory syncytial virus (RSV), Nipah virus, human papilloma virus (HPV), Herpes virus, a hepatitis virus, a hepatitis A (HepA) virus, a hepatitis B (HepB), or a hepatitis C (HepC) virus;
xi) comprises an influenza protein, or a fragment thereof;
xii) comprises an influenza protein, or a fragment thereof, wherein the influenza protein comprises a HA, NA, M1, M2, NS1, NS2, PA, PB1, or PB2 influenza protein, or a fragment thereof;
xiii) comprises an influenza protein, or a fragment thereof, wherein the influenza protein comprises an amino acid sequence that is 75.0%, 80.0%, 85.0%, 90.0%, 91.0%, 92.0%, 93.0%, 94.0%, 95.0%, 96.0%, 97.0%, 97.5%, 98.0%, 98.5%, 99.0%, 99.5%, 99.9%, 100%, or a range of percentages defined by any two of the aforementioned percentages, identical to any of SEQ ID NOs: 1-16, or a fragment thereof;
xiv) comprises an influenza protein, or a fragment thereof, wherein the influenza protein comprises an amino acid sequence that has no more than 1, 2, 3, 4, 5, 6, 7, 8, 9, 10, 20, 40, or a range defined by any of the aforementioned integers, amino acid substitutions, deletions, and/or insertions, compared to any of SEQ ID NOs: 1-16, or a fragment thereof;
xv) comprises an influenza protein, or a fragment thereof, wherein the influenza protein is encoded by a nucleic acid with a sequence that is 75.0%, 80.0%, 85.0%, 90.0%, 91.0%, 92.0%, 93.0%, 94.0%, 95.0%, 96.0%, 97.0%, 97.5%, 98.0%, 98.5%, 99.0%, 99.5%, 99.9%, 100%, or a range of percentages defined by any two of the aforementioned percentages, identical to a nucleic acid sequence encoding any of amino acid SEQ ID NOs: 1-16, or a fragment thereof
xvi) comprises an influenza protein, or a fragment thereof, wherein the influenza protein is encoded by a nucleic acid with a sequence that has no more than 1, 2, 3, 4, 5, 6, 7, 8, 9, 10, 20, 40, or a range defined by any of the aforementioned integers, nucleic acid substitutions, deletions, and/or insertions, compared to a nucleic acid sequence encoding any of amino acid SEQ ID NOs: 1-16, or a fragment thereof;
xvii) comprises a coronavirus protein, or a fragment thereof
xviii) comprises a coronavirus protein, or a fragment thereof, wherein the coronavirus comprises a severe acute respiratory syndrome coronavirus 2 (SARS-CoV-2);
xix) comprises a coronavirus protein, or a fragment thereof, wherein the coronavirus protein comprises a spike (S) protein, an envelope (E) protein, a membrane protein (M), or a nucleocapsid (N) protein;
xx) comprises a coronavirus protein, or a fragment thereof, wherein the coronavirus protein comprises 51 or S2;
xxi) comprises a coronavirus protein, or a fragment thereof, wherein the coronavirus protein comprises an amino acid sequence that is 75.0%, 80.0%, 85.0%, 90.0%, 91.0%, 92.0%, 93.0%, 94.0%, 95.0%, 96.0%, 97.0%, 97.5%, 98.0%, 98.5%, 99.0%, 99.5%, 99.9%, 100%, or a range of percentages defined by any two of the aforementioned percentages, identical to any of SEQ ID NOs: 20-29, or a fragment thereof; and/or
xxii) comprises a coronavirus protein, or a fragment thereof, wherein the coronavirus protein comprises an amino acid sequence that has no more than 1, 2, 3, 4, 5, 6, 7, 8, 9, 10, 20, 40, or a range defined by any of the aforementioned integers, amino acid substitutions, deletions, and/or insertions, compared to any of SEQ ID NOs: 20-29, or a fragment thereof

15.-35. (canceled)

36. The VLP of claim 1, wherein the anchor molecule comprises
i) a transmembrane protein, a lipid-anchored protein, or a fragment or domain thereof;
ii) a hydrophobic moiety; and/or
iii) prenylated protein, fatty acylated protein, a glycosylphosphatidylinositol-linked protein, or a fragment thereof.

37.-38. (canceled)

39. The VLP of claim 1, wherein
i) the VLP is a seVLP and the lipid bilayer is in the form of a synthetic lipid vesicle; and/or
ii) the VLP is a smVLP and the lipid bilayer is in the form of a nanodisc.

40.-45. (canceled)

46. A vaccine comprising the VLP of claim 1, and a pharmaceutically acceptable excipient, carrier, and/or adjuvant.

47. The vaccine of claim 46, wherein
i) the excipient comprises an antiadherent, a binder, a coating, a color or dye, a disintegrant, a flavor, a glidant, a lubricant, a preservative, a sorbent, a sweetener, or a vehicle;
ii) the adjuvant comprises a Toll-like receptor (TLR) agonist, imiquimod, Flt3 ligand, monophosphoryl lipid A (MLA), an immunostimulatory oligonucleotide, or a CpG oligonucleotide;
iii) the vaccine is formulated in a solvent or liquid comprising a saline solution, a dry powder, or a sugar glass;
iv) the vaccine is lyophilized;
v) the vaccine is formulated for intranasal, intradermal, intramuscular, topical, oral, subcutaneous, intraperitoneal, intravenous, or intrathecal administration;
vi) the vaccine comprises a dose of 1 pg, 10 pg, 25 pg, 100 pg, 250 pg, 500 pg, 750 pg, 1 ng, 5 ng, 10 ng, 15 ng, 20 ng, 25 ng, 50 ng, 100 ng, 250 ng, 500 ng, 1 μg, 10 μg, 50 μg, 100 μg, 500 μg, 1 mg, 5 mg, 10 mg, 50 mg, 100 mg, 500 mg, or 1 g of the vaccine, or a range of doses defined by any two of the aforementioned doses;
vii) the vaccine comprises a dose of 25 pL, 50 pL, 100 pL, 250 pL, 500 pL, 750 pL, 1 nL, 5 nL, 10 nL, 15 nL, 20 nL 25 nL, 50 nL, 100 nL, 250 nL, 500 nL, 1 μL, 10 μL, 50 μL, 100 μL, 500 μL, 1 mL, or 5 mL of the vaccine, or a range of doses defined by any two of the aforementioned doses;
viii) the vaccine is formulated for microneedle administration in a 100 pL-20 nL dose on the microneedle; and/or
ix) the vaccine further comprises a trehalose sugar glass.

48.-56. (canceled)

57. A microneedle device loaded with the vaccine of claim 46.

58. The microneedle device of claim 57, wherein
i) the microneedle device comprises a substrate comprising a sheet and a plurality of microneedles extending therefrom;
ii) the vaccine is in the form of a sugar glass; and/or
iii) the microneedle device further comprises a metal snap applicator fastened by tape to a support material.

59.-61. (canceled)

62. A method of making a seVLP, comprising:
microfluidically combining (i) an aqueous solution comprising an antigen bound to an anchor molecule with (ii) an ethanolic solution comprising a first lipid and a second lipid, thereby mixing the aqueous solution with the ethanolic solution to form a seVLP comprising a lipid bilayer comprising the first and second lipids with the anchor molecule embedded in the lipid bilayer.

63. (canceled)

64. A method for preventing, reducing the occurrence of, or reducing the severity of a disease in a subject in need thereof, comprising:
administering the vaccine of claim 46, to the subject;
wherein the administration prevents, reduces the occurrence of, or reduces the severity of the disease.

65.-77. (canceled)

78. A kit comprising a microneedle loaded with the VLP of claim 1, and a wipe, a desiccant, and/or a bandage.

79.-80. (canceled)

81. A method for determining an effectiveness of a vaccine, comprising:
obtaining a sample obtained from a subject who has been administered a vaccine, the sample comprising a presence or an amount of a virus or anti-virus antibodies;
providing a substrate comprising an angiotensin converting enzyme 2 (ACE2) or fragment thereof capable of binding to a virus protein, or a virus protein or fragment thereof capable of binding to the anti-virus antibodies;
contacting the substrate with the sample to bind virus or protein virus in the sample to the ACE2 or fragment thereof, or to bind anti-virus antibodies in the sample to the virus protein or fragment thereof;
detecting virus or protein virus bound to the ACE2 or fragment thereof, or anti-virus antibodies bound to the virus protein or fragment thereof, of the substrate; and
determining the presence or amount of the virus in the sample based on the detected virus or protein virus bound to the ACE2 or fragment thereof of the substrate, or the presence or amount of the anti-virus antibodies in the sample based on the detected anti-virus antibodies bound to the virus protein or fragment thereof of the substrate, thereby determining the effectiveness of the vaccine.

82.-100. (canceled)

101. The virus-like particle (VLP) of claim 1, comprising:
(a) a synthetic lipid bilayer comprising a first lipid and a second lipid;
(b) an anchor molecule embedded in the lipid bilayer; and
(c) a severe acute respiratory syndrome coronavirus 2 (SARS-CoV-2) protein bound to the anchor molecule.

102.-114. (canceled)

115. A vaccine comprising the VLP of claim 101, and a pharmaceutically acceptable excipient, carrier, or adjuvant.

116.-119. (canceled)

120. A vaccination method comprising administering the vaccine of claim 115 to a subject in need thereof

121. The VLP of claim 1, comprising a synthetic enveloped virus-like particle (seVLP) comprising:
(a) a synthetic lipid vesicle comprising a lipid bilayer having an inner surface and an outer surface;
(b) an anchor molecule embedded in the lipid bilayer; and
(c) a severe acute respiratory syndrome coronavirus 2 (SARS-CoV-2) protein bound to the anchor molecule.

122.-125. (canceled)

126. The VLP of claim 1, comprising a synthetic membrane virus-like particle (smVLP), comprising:
(a) a synthetic nanodisc comprising a lipid bilayer comprising an inner surface and an outer surface;
(b) an anchor molecule embedded in the lipid bilayer; and
(c) a severe acute respiratory syndrome coronavirus 2 (SARS-CoV-2) protein bound to the anchor molecule.

127.-130. (canceled)


SEQUENCE LISTING (29 sequences)

SEQ ID NO: 1 - protein, 469 aa, Influenza A virus
MNPNQKIITIGSTCMTIGMANLILQIGNIISIWVSHSIQIGNQSQIETCNQSVITYENNTWVNQTYVNISNTNFAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVDSDTVGWSWPDGAELPFTIDK

SEQ ID NO: 2 - protein, 469 aa, Influenza A virus
MNPNQKIITIGSVSLTISTICFFMQIAILITTVTLHFKQYEFNSPPNNQVMLCEPTIIERNITEIVYLTNTTIEREICPKPAEYRNWSKPQCGITGFAPFSKDNSIRLSAGGDIWVTREPYVSCDPDKCYQFALGQGTTINNVHSNNTARDRTPHRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKTDSSSSSHCLNPNNEKGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 3 - protein, 466 aa, Influenza B virus
MLPSTIQTLTLFLTSGGVLLSLYVSASLSYLLYSDILLKFSPTEITAPTMPLDCANASNVQAVNRSATKGVTLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 4 - protein, 466 aa, Influenza B virus
MLPSTIQTLTLFLTSGGVLLSLYVSASLSYLLYSDILLKFSRTEVTAPIMPLDCANASNVQAVNRSATKGVTPLLPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 5 - protein, 566 aa, Influenza A virus
MKAILVVLLYTFTTANADTLCIGYHANNSTDTVDTVLEKNVTVTHSVNLLEDKHNGKLCKLGGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDREGRMNYYWTLVEPGDKITFEATGNLVVPRYAFTMERNAGSGIIISDTPVHDCNTTCQTAEGAINTSLPFQNVHPVTIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 6 - protein, 566 aa, Influenza A virus
MKTIIALSCILCLVFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCYPYDVPDYASLRSLVASSGTLEFNNESFNWAGVTQNGTSSSCIRGSKSSFFSRLNWLTHLNSKYPALNVTMPNNEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISCFLLCVALLGFIMWACQKGNIRCNICI

SEQ ID NO: 7 - protein, 566 aa, Influenza B virus
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGKSSACIRGSSSSFFSRLNWLTHLNYTYPALNVTMPNKEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMWACQKGNIRCNICI

SEQ ID NO: 8 - protein, 584 aa, Influenza B virus
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 9 - protein, 97 aa, Influenza A virus
MSLLTEVETHTRSEWECRCSGSSDPLVIAANIIGILHLILWITDRLFFKCIYRRFKYGLKRGPSTEGVPESMREEYQQEQQSAVDVDDGHFVNIELE

SEQ ID NO: 10 - protein, 97 aa, Influenza A virus
MSLLTEVETPIRNEWGCRCNDSSDPLIVAANIIGILHLILWILDRLFFKCVCRLFKHGLKRGPSTEGVPESMREEYRKEQQNAVDADDSHFVSIELE

SEQ ID NO: 11 - protein, 109 aa, Influenza B virus
MLEPFQILTICSFILSALHFMAWTIGHLNQIKRGINMKIRIKGPNKETITREVSILRHSYQKEIQAKETMKEVLSDNMEVLNDHIIIEGLSAEEIIKMGETVLEIEELH

SEQ ID NO: 12 - protein, 109 aa, Influenza B virus
MFEPFQILSICSFILSALHFMAWTIGHLNQIKRGVNMKIRIKGPNKETINREVSILRHSYQKEIQAKEAMKEVLSDNMEVLSDHIVIEGLSAEEIIKMGETVLEVEESH

SEQ ID NO: 13 - protein, 100 aa, Influenza B virus
MNNATFNYTNVNPISHIRGSIIITICVSFIIILTILGYIAKILTNRNNCTNNAIGLCKRIKCSGCEPFCNKRGDTSSPRTGVDIPAFILPGLNLSESTPN

SEQ ID NO: 14 - protein, 100 aa, Influenza B virus
MNNATFNYTNVNLISHIRGSVIITICVSFIVILTIFGYIAKIFTNRSNCTNNAIGLCKRIKCSGCEPFCNKRGDTSSPRTGVDVPSFILPGLNLSESTPN

SEQ ID NO: 15 - protein, 594 aa, Influenza B virus
MKAIIVLLMVVTSSADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEGAPGGPYKIGTSGSCPNITNGNGFFATMAWAVPDKNKTATNPLTIEVPYVCTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDKIAAGTFDAGEFSLPTFDSLNITAASLNDDGLDNHTILLYYSTAASSLAVTLMIAIFVVYMVSRDNVSCSICLVPRGSHHHHHH

SEQ ID NO: 16 - DNA, 1788 bp, Influenza B virus
atgaaggccatcatcgtgcttctcatggtggtgaccagctcagcggaccggatctgcaccggcattaccagctccaactccccccacgtcgtgaaaactgcgacccagggagaagtgaacgtcactggcgtgattccgctgaccaccacccccaccaagtcccatttcgccaacctgaaggggaccgaaacacggggcaaactctgcccgaagtgcctgaactgtaccgatctggacgtggcactgggaaggccaaagtgcaccgggaagattccgagcgccagagtgtcgatcttgcacgaagtcagacctgtgacctcgggatgtttccccattatgcacgaccggacaaagatccgccagctccctaatctgttgcggggatatgagcacgtccgcctttcgactcacaacgtgatcaacgccgaaggcgcacctggtggtccttacaagatcgggacttcgggttcctgcccgaacatcaccaacggaaacggctttttcgccaccatggcctgggctgtgccagacaagaacaagactgccaccaatcccctgaccatcgaagtgccgtacgtgtgcacggagggggaagatcagattactgtgtgggggttccacagcgataacgaaacccagatggccaagctgtacggagattcaaagccccagaaattcacttcgagcgctaacggtgtcaccactcactacgtgtcccaaatcggagggttcccgaatcaaaccgaggacgggggattgccgcaatccggtcgcatcgtggtcgactatatggtgcagaagtcgggcaaaactggcactatcacgtaccagaggggaatcctgctgcctcaaaaagtgtggtgtgcgtcaggccggtctaaggtcatcaagggttccctgcccctcatcggagaggccgactgcctccacgaaaaatacggaggcctcaacaagtccaagccctactacaccggggaacatgccaaggccatcgggaactgccccatttgggttaagaccccactgaagctcgccaacggcactaagtacagacctccggccaagttgctgaaggaacggggatttttcggagccattgcgggattcctggaaggaggctgggagggaatgattgcggggtggcacggatacactagccatggcgctcacggagtggcagtggcggcagacctgaagtccactcaggaggccatcaacaagattaccaagaacctgaacagcctgtccgagctggaagtcaagaatctccagaggctcagcggcgctatggacgagcttcataatgagatcctggagctggatgagaaggtcgacgatctccgcgcggacaccataagctcgcagatcgagctggccgtgcttctgtcgaacgagggcatcatcaactccgaggacgagcacctcctggcacttgaacggaagctcaagaaaatgctgggaccttccgctgtggaaattggcaacggctgcttcgagactaagcacaagtgcaaccagacgtgcctggataagattgccgccggaaccttcgacgccggagagtttagcctgcccaccttcgactccctgaacatcaccgcggcctcactgaatgatgacggccttgataaccacaccatcctcctgtactactccaccgccgcatcctcactcgccgtgactctgatgatcgccatcttcgtggtgtacatggtcagccgcgacaacgtgtcctgttccatttgcctggtgccgagaggttcccaccatcatcaccatcactaatga

SEQ ID NO: 17 - protein, 7 aa, Artificial Sequence
  Description of Artificial Sequence Synthetic peptide
YVADAPK

SEQ ID NO: 18 - protein, 80 aa, Homo sapiens
MSSSSWLLLSLVAVTAAQSTIEEQAKTFLDKFNHEAEDLFYQSSLASWNYNTNITEENVQNMNNAGDKWSAFLKEQSTLA

SEQ ID NO: 19 - protein, 805 aa, Homo sapiens [sequence omitted]

SEQ ID NO: 20 - protein, 38 aa, Severe acute respiratory syndrome coronavirus 2
MGYINVFAFPFTIYSLLLCRMNSRNYIAQVDVVNFNLT

SEQ ID NO: 21 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 22 - protein, 61 aa, Severe acute respiratory syndrome coronavirus 2
MFHLVDFQVTIAEILLIIMRTFKVSIWNLDYIINLIIKNLSKSLTENKYSQLDEEQPMEID

SEQ ID NO: 23 - protein, 7096 aa, Severe acute respiratory syndrome coronavirus 2 [sequence omitted]

SEQ ID NO: 24 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 25 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 26 - protein, 275 aa, Severe acute respiratory syndrome coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 27 - protein, 121 aa, Severe acute respiratory syndrome coronavirus 2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 28 - protein, 121 aa, Severe acute respiratory syndrome coronavirus 2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 29 - protein, 75 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV